(19) United States
(12) Patent Application Publication
(10) Pub. No.: US 2009/0068255 A1
(43) Pub. Date: Mar. 12, 2009

(54) USE OF MATRIX METALLOPROTEINASE INHIBITORS IN SKIN CARE

(76) Inventors: Betty Yu, Cambridge, MA (US); Amir Nashat, Newton, MA (US); Daniel Griffith Anderson, Sudbury, MA (US); David Thomas Puerta, Melrose, MA (US); Benjamin Adams, Cambridge, MA (US); Scott Clark, Pittsfield, MA (US); Yushan Kim, Cambridge, MA (US); Eric George Spengler, Ridgefield, CT (US); Ronald P. McLaughlin, Reading, MA (US); Susan Eilidh Bedford, Carlisle, MA (US); Zhi Li, San Diego, CA (US)

Correspondence Address: FITZPATRICK CELLA HARPER & SCINTO, 30 ROCKEFELLER PLAZA, NEW YORK, NY, 10112, US

(21) Appl. No.: 12/112,374

(22) Filed: Apr. 30, 2008

Related U.S. Application Data
(60) Provisional application No. 60/914,873, filed on Apr. 30, 2007.

(51) Int. Cl. A61K 8/14; A61K 8/49; A61K 31/351; A61Q 19/08; A61Q 17/04; A61P 17/02; A61P 35/00; A61P 17/00; A61Q 19/00; A61K 31/4412; A61K 9/127

(52) U.S. Cl. 424/450; 514/459; 514/350; 424/59

(57) ABSTRACT

The application of matrix metalloproteinase (MMP) inhibitors to the skin inhibits the degradation of proteins found in the skin including collagen, elastin, and other basement membrane and extracellular matrix protein. MMP inhibitors may be used in both cosmetic compositions and pharmaceutical compositions for application to skin. MMP inhibitors are formulated with a cosmetically suitable vehicle or pharmaceutically acceptable excipient for application to the skin as creams, lotions, ointments, solutions, face masks, etc. As cosmetics, the inventive MMP inhibitor compositions are applied to the skin to prevent or reduce the appearance of wrinkles, pigmentation changes, loss of elasticity, or other effects associated with aging or sun damage. As pharmaceuticals, the inventive MMP inhibitor compositions may also be applied to the skin to treat or prevent a skin disease (e.g., proliferative disease, inflammatory disease).

### RELATED APPLICATIONS

The present invention claims priority under 35 U.S.C. § 119(e) to U.S. provisional patent application, U.S. Ser. No. 60/914,873, filed Apr. 30, 2007, which is incorporated herein by reference.

### BACKGROUND OF THE INVENTION

The skin is the largest organ of the human body and extends over the entire body. The skin functions primarily to protect us from the outside world. The skin also functions to regulate the temperature of the body, protects the body from harmful UV rays, provides a defense against pathogens, stores fat, provides the sense the touch, excretes waste, synthesizes vitamin D, and provides cushioning and attachment. In protecting us from the outside world, the skin is constantly exposed to harsh temperatures, sunlight, dirt, dust, wind, chemicals, pathogens, and other insults. In addition, the skin is routinely subjected to washing and shaving.

Skin is composed of two major layers: the epidermis and the underlying dermis, which are distinct in terms of their architecture, physiology, and function. The epidermis is a stratified epithelium composed of four layers: the stratum basale, stratum spinosum, stratum granulosum, and the outermost stratum corneum. The stratum basale contains a single layer of cuboidal keratinocytes attached to a basement membrane. Above this layer is the spinous layer, characterized by presence of numerous desmosomes. The stratum granulosum overlies the stratum spinosum and consists of keratinocytes that contain basophilic granules of keratohyalin as well as lamellar granules in the intercellular compartment. The stratum corneum is the most superficial layer and is composed of anucleated, flattened, fully keratinized cells (corneocytes) fused together to form a plate-like structure. The intercellular space is occupied by ordered lipid lamellae that contain specialized proteins and lipids, such as ceramides, fatty acids, and cholesterols, which are secreted from lamellar bodies in the stratum granulosum. The resulting “bricks and mortar” structure provides the stratum corneum with the ability to perform its protective and moisture retaining functions. The thickness of the epidermis ranges from about 75 to 150 μm except on the soles and palms, where it is about 0.4 to 0.6 mm. The dermoepidermal junction (DEJ) is an undulating basement membrane composed primarily of collagen that separates the epidermis from the dermis.

The dermis is a dense, fibroelastic connective tissue that lies beneath the epidermis and provides a strong and flexible supporting layer. It is composed of cells (e.g., fibroblasts), ground substance, and a fibrous network containing collagenous and elastic fibers and also contains blood vessels, nerves, hair follicles, smooth muscle, glands and lymphatic tissue. Collagen, primarily types I, III, V, and VI, forms the majority of the fibrous component, making up about 75% of the dry weight of the dermis and imparting firmness and tensile strength.

The dermis can be divided into two regions. The papillary dermis conforms to the shape of the overlying epidermis. The reticular dermis lies below the papillary dermis and forms the majority of the dermal layer, giving it most of its elasticity and strength. Elastic fibers of the papillary dermis are oriented parallel (elaunin fibers) or perpendicular (oxytalan fibers) to the DEJ and are thinner than the elastic fibers of the reticular dermis. Oxytalan fibers lack the elastin core while elaunin fibers contain a small amount of elastin. Mature elastin fibers are found primarily arranged in bundles in the reticular dermis and measure about 1-3 μm in diameter.

Aging and exposure to environmental insults affect the appearance and structure of the skin. Sun-protected, naturally aged skin exhibits epidermal and dermal thinning, fragility, and fine, shallow wrinkles. There is a loss of elastic fibers in the papillary dermis, and collagen fibers become increasingly dense and more randomly oriented. Exposure to ultraviolet radiation from the sun accelerates and alters the degenerative processes associated with aging, resulting in a constellation of changes collectively known as photodamage. As much as 80% of facial aging may well be attributable to sun exposure. Photodamaged skin is characterized by loss of elasticity, deep wrinkles, increased roughness and dryness, and altered pigmentation (age spots). The skin may assume a leathery, thickened appearance characterized by deep furrows.

Many individuals desire to preserve a youthful appearance. At the same time, tanned skin is considered attractive and outdoor recreational activities are popular, resulting in significant sun exposure and consequent photodamage to skin. Various approaches have been developed or are under investigation to reduce the visible signs of photodamage (Stern, R., N. Engl. J. Med, 350:1526-1534, 2004). Use of sunscreens can help to prevent further damage. Topical retinoids (vitamin A derivatives) can reduce the severity of photoaging (U.S. Pat. No. 4,877,805; incorporated herein by reference). It has been proposed to treat the skin with compositions containing inhibitors of collagenase or elastase (U.S. Pat. Nos. 5,614,489; 6,884,425; each of which is incorporated herein by reference). Chemical peels, which involve application of a variety of different chemical compounds to skin, result in temporary improvement in the appearance of photodamaged skin in some individuals. Microdermabrasion and laser resurfacing are other alternatives. Injection of botulinum toxin (e.g., Botox®) has gained popularity as a means of reducing furrows caused by muscle hypertonicity. Skin fillers such as bovine collagen and hyaluronic acid are used for soft tissue augmentation to reduce the appearance of, or to treat deep wrinkles.

Such factors as exposure to the sun contribute to the premature aging of skin contributing to the formation of lines and wrinkles, skin dryness, skin fading, roughness, hyperpigmentation, age spots, and loss of elasticity. The physiological and pathological degradation of the connective tissue component of the skin by proteases from resident cells contributes to the loss of elasticity of the skin. A major class of enzymes involved in this process is the matrix metalloproteinases (MMPs).

MMPs are zinc(II)-containing hydrolytic enzymes involved in the breakdown of components of the extracellular matrix (ECM) and basement membrane such as aggrecan, collagen, elastin, fibronectin, gelatin, and laminin. The ability of MMPs to degrade components of the ECM is essential to cell growth, cell division, bone growth, wound healing, embryogenesis, and angiogenesis. The MMPs are divided into several different classes. Over 10 different members have been identified to date. They are referred to numerically as MMP-1, MMP-2, etc. as well as by a common name. The MMPs share several structural and functional properties but differ in their substrate specificities. Examples of MMPs include collagenase I (MMP-1, fibroblast collagenase; EC 3.4.24.3); collagenase II (MMP-8, neutrophil collagenase; EC 3.4.24.34); collagenase III (MMP-13); proteoglycanase, matrilysin (MMP-7); gelatinase A (MMP-2, 72 kDa gelatinase, basement membrane collagenase; EC3.4.24.24); stromelysin-3 (MMP-11); gelatinase B (MMP-12, HME, human macrophage elastase); and membrane MMP (MMP-14).

Given the potential use of MMP inhibitors on skin for cosmetic or pharmaceutical purposes, compositions for delivering an MMP inhibitor to the skin are needed. Since many MMP inhibitors are poorly soluble in pharmaceutical and cosmetic excipients, systems for solubilizing and formulating these important new compounds are needed.

### SUMMARY OF THE INVENTION

The present invention provides a system for treating or caring for skin using MMP inhibitors. The presence of an MMP inhibitor in a skin care system prevents the degradation of proteins of the ECM and basement membrane responsible for making the skin elastic and youthful appearing and feeling. Specifically, the MMP inhibitor inhibits the MMPs released from cells found in the skin, including inflammatory cells. The inventive skin care system is particularly useful in caring for human skin, in particular, facial skin, skin of the head and neck, or skin of any other part of the body. The invention provides cosmetic methods for topically administering MMP inhibitors for use in skin care and provides cosmetic compositions which include a cosmetically effective amount of an MMP inhibitor. In addition, the invention also provides therapeutic methods for topically administering MMP inhibitors for use in treating skin conditions or diseases and provides pharmaceutical compositions which include a therapeutically effective amount of an MMP inhibitor. Inventive compositions include, but are not limited to, lotions, creams, gels, pastes, serums, sticks, powders, sprays, foams, solutions, ointments, face masks, and patches. The invention also provides new small molecules which are useful inhibitors of MMPs (e.g., in the treatment and care of skin). MMP inhibitors are particularly useful in treating and caring for sun-damaged skin and/or aged skin. MMP inhibitors are also useful in preventing and reducing the signs of aging or sun damage. The inventive cosmetic compositions are particularly useful in improving the appearance of skin. Without wishing to be bound by any particular theory, the use of MMP inhibitors on skin is thought to work by decreasing the degradation of collagen in the treated skin, increase the levels of collagen and/or procollagen in the skin, prevent the decrease in levels of collagen and/or procollagen in the skin, and/or stimulate the formation of new collagen or new collagen in the skin. The levels of other extracellular matrix proteins (e.g., elastin, fibronectin, laminins) in the skin may also be increased or maintained using MMP inhibitors. MMP inhibitors may also be used in wound healing and/or in treating inflammatory diseases, autoimmune diseases, and/or proliferative diseases such as cancer. The inventive pharmaceutical compositions are particularly useful in treating diseases associated with the skin.

In one aspect, the invention provides methods of administering an MMP inhibitor to the skin of a subject. The invention provides for both therapeutic and cosmetic uses of MMP inhibitors. A cosmetically effective amount or therapeutically effective amount of an MMP inhibitor is administered topically to the skin of a subject (e.g., human). The MMP inhibitor may be administered to the skin in the form of a cream, lotion, ointment, powder, spray, solution, gel, paste, serum, stick, foam, patch, face masks, etc. The MMP inhibitor may also be administered in a semi-solid dispersed system such as a nonionic, anionic, cationic, or gel network emulsion. Such an emulsion may be oil in water, water in oil, silicone in water, or water in silicone. For therapeutic purposes, the MMP inhibitors may be administered using techniques specifically designed to deliver pharmaceutical agents to the skin, for example, microneedle systems, iontophoresis, electroporation, and ultrasound. The administration may be repeated in order to achieve the desired effect. In certain embodiments, the MMP inhibitor or composition thereof is administered at least once a day. The administration of the MMP inhibitor or compositions thereof may be continued for days, weeks, months, or indefinitely. An MMP inhibitor may also be administered to the skin of a subject (e.g., human) to prevent or lessen the appearance of age-associated features such as wrinkles, lines, changes in pigmentation, loss of elasticity, redness, etc. The MMP inhibitor may be administered to a portion of the skin such as the face or to the entire body. Any MMP inhibitor known in the art may be utilized in the present invention including those described in U.S. Pat. Nos. 4,877,805; 5,837,224; 6,365,630; 6,630,516; 6,683,069; 6,919,072; 6,942,870; and 7,176,217; published international PCT applications WO 05/1103399 and WO 06/028523; and U.S. provisional applications, U.S. Ser. No. 60/566,882, filed Apr. 29, 2004; U.S. Ser. No. 60/576,444, filed Jun. 3, 2004; and U.S. Ser. No. 60/826,488, filed Sep. 21, 2006; each of which is incorporated herein by reference. In certain embodiments, the MMP inhibitor is of one of the formulae:

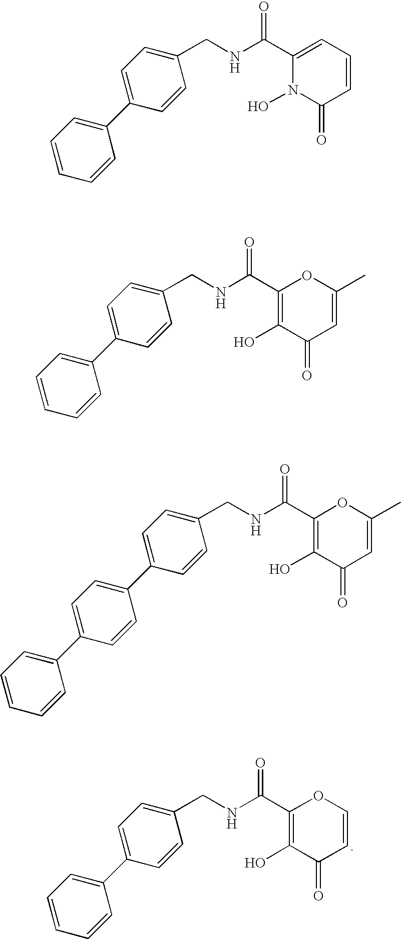

Cosmetic compositions that include MMP inhibitors are also provided. The cosmetic composition typically includes a cosmetically effective amount of an MMP inhibitor for topical administration. The cosmetic composition includes the MMP inhibitor and a cosmetically acceptable vehicle (e.g., an oil, emollient, lubricant, butter, wax, surfactants, detergents, emulsifier, water, solubilizer, solvent, fatty acid, thickener, polymer, resin, preservative, etc.). The cosmetic compositions may also include other agents such as, for example, sunscreens, antioxidants, fragrances, perfumes, plant extracts, proteins, amino acids, carbohydrates, coloring agents, preservatives, pharmaceutical agents, vitamins, humectants, film former, pH adjusting agent, buffers, neutralizing agents, salts, etc. In certain embodiments, the cosmetic composition is formulated such that the active ingredient, MMP inhibitor, penetrates one or more layers of the skin. In certain embodiments, the cosmetic composition is formulated such that the active ingredient(s) penetrates only the outermost layer of skin. Topical delivery of the MMP inhibitor may be enhanced through the use of film-forming agents (e.g., PVP, acrylates, acrylamides, and co-polymers thereof). The cosmetic composition may be a cream, lotion, emulsion, solution, gel, spray, powder, ointment, foam, solution, stick, face masks, etc. for administration of an MMP inhibitor to the skin. The composition may be a solution, suspension, mixture, emulsion, or other combination of components. The inventive compositions may include particles (e.g., nanoparticles, microparticles, liposomes, micelles) which include the MMP inhibitor or other components of the composition. In certain embodiments, the MMP inhibitor being used in the composition is difficult to solubilize, and the cosmetic composition includes a solubilizer such as dimethylisosorbide, polyethylene glycol, triethanolamine, phospholipids, and quaternary amines. Useful solubilizers include ethers, alkoxylated alcohols, alkoxylated amines, sorbitan esters, phospholipids, and fatty quaternaries. Without wishing to be bound by any particular theory, it is generally thought that the solubilizer does not sterically exclude the MMP inhibitor from interacting with alkoxylated moieties of the solubilizer. Such delivery vehicles may allow for the extended release or timed release of the MMP inhibitor or other component. The MMP inhibitor may comprise approximately 0.0001-30% by weight of the cosmetic compositions, preferably 0.001-10% by weight of the cosmetic composition. In certain embodiments, the MMP inhibitor is approximately 0.01-3% by weight of the cosmetic composition. The invention also provides kits including the inventive cosmetic compositions and instructions for using the composition. The kit may also include other skin care products (e.g., cleansers, lotions, sunscreens, moisturizers, cosmetics, etc.) and/or applicators for cosmetic compositions included in the kit.

Pharmaceutical compositions that include MMP inhibitors are also provided. The pharmaceutical composition typically includes a therapeutically effective amount of an MMP inhibitor for topical administration. The pharmaceutical composition includes the MMP inhibitor and a pharmaceutically acceptable excipient. The pharmaceutical composition is formulated such that the active ingredient, MMP inhibitor, can penetrate the various layers of the skin. Topical delivery of the MMP inhibitor may be enhanced through the use of film-forming agents (e.g., PVP, acrylates, acrylamides, and co-polymers thereof). The pharmaceutical composition may be a cream, lotion, emulsion, solution, cream, gel, paste, stick, foam, spray, serum, powder, etc. for administration of an MMP inhibitor to the skin. The composition may be a solution, suspension, mixture, emulsion, or other combination of components. The inventive compositions may include particles (e.g., nanoparticles, microparticles, liposomes, micelles) which include the MMP inhibitor or other components of the composition. In certain embodiments, the MMP inhibitor is difficult to solubilize, and the pharmaceutical composition may include a solubilizer such as dimethylisosorbide, polyethylene glycol, triethanolamine, phospholipids, and quaternary amines. Useful solubilizers include ethers, alkoxylated alcohols, alkoxylated amines, sorbitan esters, phospholipids, and fatty quaternaries. The delivery vehicle(s) may allow for extended or timed release of the MMP inhibitor or other component. In certain embodiments, the pharmaceutical composition is designed for iontophoresis, electroporation, injection, or delivery by ultrasound of the MMP inhibitor into skin. The MMP inhibitor may comprise approximately 0.0001-30% by weight of the pharmaceutical composition, preferably 0.001-10% by weight of the pharmaceutical composition. In certain embodiments, the MMP inhibitor is approximately 0.01-3% by weight of the pharmaceutical composition. The invention also provides kits including the inventive pharmaceutical compositions and instructions for using the composition. The kit may include multiple unit dosages of the MMP inhibitor. For example, the kit may include a month supply of the MMP inhibitor composition.

The invention also provides novel MMP inhibitors based on the following formulae:

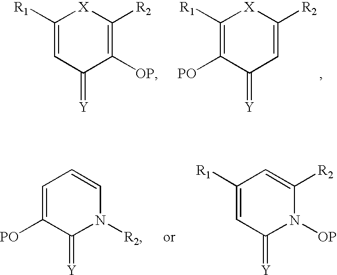

wherein

X is O or NR′, wherein R′ is hydrogen, C₁-C₆ alkyl, or a nitrogen protecting group;

Y is O or S;

P is hydrogen or an oxygen protecting group;

R₁ is hydrogen or C₁-C₆ alkyl; and

R₂ is an aryl- or heteroaryl-containing moiety which may be optionally substituted. In certain embodiments, R₂ includes one or more substituted phenyl rings. The phenyl rings may be linked together by covalent bonds or through an aliphatic or heteroaliphatic linker, which may be optionally substituted. In certain embodiments, R₁ is hydrogen or methyl. In certain embodiments, P is hydrogen. The compounds are particularly useful in inhibiting MMPs found in the skin. In certain embodiments, the compounds specifically inhibit one or more classes of MMPs. In certain embodiments, the compounds have an IC₅₀ below 10 μM, below 1 μM, below 0.1 μM, below 0.01 μM, or below 0.001 μM for inhibiting an MMP in a standard assay. Salts, pro-drugs, stereoisomers, tautomers, and other cosmetically acceptable forms of these compounds may be used in cosmetic compositions for skin care or pharmaceutical compositions.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in inhibiting MMPs, particularly those found in the skin. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term acyl as used herein refers to a group having the general formula —C(═O)R, where R is alkyl, alkenyl, alkynyl, aryl, carbocylic, heterocyclic, or aromatic heterocyclic. An example of an acyl group is acetyl.

The term aliphatic, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

The term alkyl as used herein refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-10 carbon atoms. In another embodiment, the alkyl group employed contains 1-8 carbon atoms. In still other embodiments, the alkyl group contains 1-6 carbon atoms. In yet another embodiments, the alkyl group contains 1-4 carbons. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substituents.

The term alkoxy as used herein refers to a saturated (i.e., alkyl-O—) or unsaturated (i.e., alkenyl-O— and alkynyl-O—) group attached to the parent molecular moiety through an oxygen atom. In certain embodiments, the alkyl group contains 1-20 aliphatic carbon atoms. In certain other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples include, but are not limited to, methoxy, ethoxy, propoxy, isopropoxy, n-butoxy, tert-butoxy, i-butoxy, sec-butoxy, neopentoxy, n-hexoxy, and the like.

The term alkenyl denotes a monovalent group derived from a hydrocarbon moiety having at least one carbon-carbon double bond by the removal of a single hydrogen atom. In certain embodiments, the alkenyl group employed in the invention contains 1-20 carbon atoms. In some embodiments, the alkenyl group employed in the invention contains 1-10 carbon atoms. In another embodiment, the alkenyl group employed contains 1-8 carbon atoms. In still other embodiments, the alkenyl group contains 1-6 carbon atoms. In yet another embodiments, the alkenyl group contains 1-4 carbons. Alkenyl groups include, for example, ethenyl, propenyl, butenyl, 1-methyl-2-buten-1-yl, and the like.

The term alkynyl as used herein refers to a monovalent group derived form a hydrocarbon having at least one carbon-carbon triple bond by the removal of a single hydrogen atom. In certain embodiments, the alkynyl group employed in the invention contains 1-20 carbon atoms. In some embodiments, the alkynyl group employed in the invention contains 1-10 carbon atoms. In another embodiment, the alkynyl group employed contains 1-8 carbon atoms. In still other embodiments, the alkynyl group contains 1-6 carbon atoms. Representative alkynyl groups include, but are not limited to, ethynyl, 2-propynyl (propargyl), 1-propynyl, and the like.

The term alkylamino, dialkylamino, and trialkylamino as used herein refers to one, two, or three, respectively, alkyl groups, as previously defined, attached to the parent molecular moiety through a nitrogen atom. The term alkylamino refers to a group having the structure —NHR′ wherein R′ is an alkyl group, as previously defined; and the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. In certain embodiments, the alkyl group contain 1-20 aliphatic carbon atoms. In certain other embodiments, the alkyl group contains 1-10 aliphatic carbon atoms. In yet other embodiments, the alkyl group contains 1-8 aliphatic carbon atoms. In still other embodiments, the alkyl group contain 1-6 aliphatic carbon atoms. In yet other embodiments, the alkyl group contain 1-4 aliphatic carbon atoms. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, iso-propylamino, piperidino, trimethylamino, and propylamino.

In general, the terms aryl and heteroaryl, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substituents, i.e., the substituents recited for aliphatic moieties, or for other moieties as disclosed herein, resulting in the formation of a stable compound. In certain embodiments of the present invention, aryl refers to a mono- or bicyclic carbocyclic ring system having one or two aromatic rings including, but not limited to, phenyl, naphthyl, tetrahydronaphthyl, indanyl, indenyl, and the like. In certain embodiments of the present invention, the term heteroaryl, as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The term carboxylic acid as used herein refers to a group of formula —CO₂H.

The terms halo and halogen as used herein refer to an atom selected from fluorine, chlorine, bromine, and iodine.

The term heteroaliphatic, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term heterocyclic, as used herein, refers to an aromatic or non-aromatic, partially unsaturated or fully saturated, 3- to 10-membered ring system, which includes single rings of 3 to 8 atoms in size and bi- and tri-cyclic ring systems which may include aromatic five- or six-membered aryl or aromatic heterocyclic groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring.

The term aromatic heterocyclic, as used herein, refers to a cyclic aromatic radical having from five to ten ring atoms of which one ring atom is selected from sulfur, oxygen, and nitrogen; zero, one, or two ring atoms are additional heteroatoms independently selected from sulfur, oxygen, and nitrogen; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like. Aromatic heterocyclic groups can be unsubstituted or substituted with substituents selected from the group consisting of branched and unbranched alkyl, alkenyl, alkynyl, haloalkyl, alkoxy, thioalkoxy, amino, alkylamino, dialkylamino, trialkylamino, acylamino, cyano, hydroxy, halo, mercapto, nitro, carboxyaldehyde, carboxy, alkoxycarbonyl, and carboxamide.

Specific heterocyclic and aromatic heterocyclic groups that may be included in the compounds of the invention include: 3-methyl-4-(3-methylphenyl)piperazine, 3 methylpiperidine, 4-(bis-(4-fluorophenyl)methyl)piperazine, 4-(diphenylmethyl)piperazine, 4-(ethoxycarbonyl)piperazine, 4-(ethoxycarbonylmethyl)piperazine, 4-(phenylmethyl)piperazine, 4-(1-phenylethyl)piperazine, 4-(1,1-dimethylethoxycarbonyl)piperazine, 4-(2-(bis-(2-propenyl)amino)ethyl)piperazine, 4-(2-(diethylamino)ethyl)piperazine, 4-(2-chlorophenyl)piperazine, 4-(2-cyanophenyl)piperazine, 4-(2-ethoxyphenyl)piperazine, 4-(2-ethylphenyl)piperazine, 4-(2-fluorophenyl)piperazine, 4-(2-hydroxyethyl)piperazine, 4-(2-methoxyethyl)piperazine, 4-(2-methoxyphenyl)piperazine, 4-(2-methylphenyl)piperazine, 4-(2-methylthiophenyl)piperazine, 4-(2-nitrophenyl)piperazine, 4-(2-nitrophenyl)piperazine, 4-(2-phenylethyl)piperazine, 4-(2-pyridyl)piperazine, 4-(2-pyrimidinyl)piperazine, 4-(2,3-dimethylphenyl)piperazine, 4-(2,4-difluorophenyl)piperazine, 4-(2,4-dimethoxyphenyl)piperazine, 4-(2,4-dimethylphenyl)piperazine, 4-(2,5-dimethylphenyl)piperazine, 4-(2,6-dimethylphenyl)piperazine, 4-(3-chlorophenyl)piperazine, 4-(3-methylphenyl)piperazine, 4-(3-trifluoromethylphenyl)piperazine, 4-(3,4-dichlorophenyl)piperazine, 4-3, 4-dimethoxyphenyl)piperazine, 4-(3,4-dimethylphenyl)piperazine, 4-(3,4-methylenedioxyphenyl)piperazine, 4-(3,4,5-trimethoxyphenyl)piperazine, 4-(3,5-dichlorophenyl)piperazine, 4-(3,5-dimethoxyphenyl)piperazine, 4-(4-(phenylmethoxy)phenyl)piperazine, 4-(4-(3,1-dimethylethyl)phenylmethyl)piperazine, 4-(4-chloro-3-trifluoromethylphenyl)piperazine, 4-(4-chlorophenyl)-3-methylpiperazine, 4-(4-chlorophenyl)piperazine, 4-(4-chlorophenyl)piperazine, 4-(4-chlorophenylmethyl)piperazine, 4-(4-fluorophenyl)piperazine, 4-(4-methoxyphenyl)piperazine, 4-(4-methylphenyl)piperazine, 4-(4-nitrophenyl)piperazine, 4-(4-trifluoromethylphenyl)piperazine, 4-cyclohexylpiperazine, 4-ethylpiperazine, 4-hydroxy-4-(4-chlorophenyl)methylpiperidine, 4-hydroxy-4-phenylpiperidine, 4-hydroxypyrrolidine, 4-methylpiperazine, 4-phenylpiperazine, 4-piperidinylpiperazine, 4-(2-furanyl)carbonyl)piperazine, 4-((1,3-dioxolan-5-yl)methyl)piperazine, 6-fluoro-1,2,3,4-tetrahydro-2-methylquinoline, 1,4-diazacylcloheptane, 2,3-dihydroindolyl, 3,3-dimethylpiperidine, 4,4-ethylenedioxypiperidine, 1,2,3,4-tetrahydroisoquinoline, 1,2,3,4-tetrahydroquinoline, azacyclooctane, decahydroquinoline, piperazine, piperidine, pyrrolidine, thiomorpholine, and triazole.

The term carbamoyl or carbamyl, as used herein, refers to an amide group of the formula —CONH₂.

The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents may also be further substituted (e.g., an aryl group substituent may have another substituent off it, such as another aryl group, which is further substituted with fluorine at one or more positions).

The term thiol, as used herein, refers to a group of the formula —SH.

The following are more general terms used throughout the present application:

As used herein and in the claims, the singular forms “a”, “an”, and “the” include the plural reference unless the context clearly indicates otherwise. Thus, for example, a reference to “a compound” includes a plurality of such compounds.

“Animal”: As used herein, the term “animal” refers to any member of the animal kingdom. In some embodiments, “animal” refers to a human, at any stage of development. In some embodiments, “animal” refers to a non-human animal, at any stage of development. In some embodiments, animals include, but are not limited to, mammals, birds, reptiles, amphibians, fish, and/or worms. In certain embodiments, the non-human animal is a mammal (e.g., a rodent, a mouse, a rat, a rabbit, a monkey, a dog, a cat, a sheep, cattle, a primate, and/or a pig). In certain embodiments, the animal has at least a portion of its body which is hairless such as a human or hairless breed of dog or cat. In some embodiments, an animal may be a transgenic animal, genetically-engineered animal, and/or a clone.

“Cosmetically effective amount”: As used herein, the term “cosmetically effective amount” means an amount of an MMP inhibitor that is sufficient, when administered to a subject, to impart a desired characteristic (e.g., appearance, attractiveness, feeling) on the skin or hair of the subject.

“Effective amount”: In general, the “effective amount” of an active agent such as an MMP inhibitor refers to an amount sufficient to elicit the desired biological, pharmaceutical, therapeutic, or cosmetic result. As will be appreciated by those of ordinary skill in this art, the effective amount of an MMP inhibitor may vary depending on such factors as the desired endpoint, the pharmacokinetics of the compound, the skin condition being treated, the mode of administration, the formulation of the agent, and the subject. For example, the effective amount of an MMP inhibitor is the amount that results in decreased signs and/or appearance of aging and/or sun damage. In certain embodiments, the effective amount of an MMP inhibitor is the amount sufficient to promote wound healing.

“Film-forming agent”: The term “film-forming agent” as used herein refers to an agent that when applied to skin leaves a pliable, cohesive, and continuous covering of the skin or hair. The created film may be hydrophilic and may leave the skin with a smooth feel. Exemplary film-forming agents include cellulose and derivatives thereof, polyvinyl alcohol, polyethylene, PVP, acrylates, acrylamides, and co-polymers thereof.

“Polynucleotide” or “oligonucleotide”: The terms “polynucleotide” or “oligonucleotide” refer to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′-N-phosphoramidite linkages).

“Peptide” or “protein”: According to the present invention, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, an acyl group (e.g., acetyl group), a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

The terms “saccharide”, “polysaccharide”, “carbohydrate”, and “oligosaccharide”, may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replace with a fluorine, or N-acetylglucosamine, a nitrogen-containing form of glucose. (e.g., 2′-fluororibose, deoxyribose, and hexose). Carbohydrates may exist in many different forms, for example, conformers, cyclic forms, acyclic forms, stereoisomers, tautomers, anomers, and isomers.

“Small molecule”: As used herein, the term “small molecule” is used to refer to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, heterocyclic rings, etc.). In some embodiments, small molecules are monomeric and have a molecular weight of less than about 1500 g/mol. In certain embodiments, the molecular weight of the small molecule is less than about 1000 g/mol or less than about 500 g/mol. Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference.

“Therapeutically effective amount” or “pharmaceutically effective amount”: As used herein, the term “therapeutically effective amount” or “pharmaceutically effective amount” means an amount of an MMP inhibitor that is sufficient, when administered to a subject suffering from or susceptible to a disease, disorder, and/or condition, to treat, prevent, and/or diagnose the disease, disorder, and/or condition.

### BRIEF DESCRIPTION OF THE DRAWING

FIG. 1 shows the change from baseline over 16 weeks of the appearance of telangiectasia (redness) in the group treated with the MMP inhibitor versus placebo.

FIG. 2 shows the trending toward improvement in the appearance of coarse wrinkles upon treatment with the MMP inhibitor over 16 weeks.

FIG. 3 shows the trending toward improvement in the appearance of pore size upon treatment with the MMP inhibitor over 16 weeks.

FIG. 4 shows percent subject improvement in target attributes with treatment using the MMP inhibitor over 16 weeks

FIG. 5 shows that profilometry of silicon replicas was consistent with the improvement of coarse wrinkle scores.

### DETAILED DESCRIPTION OF CERTAIN EMBODIMENTS OF THE INVENTION

The present invention provides a skin care system based on the discovery that MMP inhibitors prevent or at least reduce the signs of aging and/or sun damage in skin. Novel MMP inhibitors are also provided for use in skin care. Cosmetic compositions, pharmaceutical compositions, and kits including MMP inhibitors and methods of using such compositions in the care and treatment of skin are also provided. The cosmetic compositions typically comprise a cosmetically effective amount of an MMP inhibitor and a cosmetically acceptable vehicle. The pharmaceutical compositions typically comprise a therapeutically effective amount of an MMP inhibitor and a pharmaceutically acceptable excipient. The cosmetic or pharmaceutical compositions may be lotions, creams, gels, pastes, serums, sticks, powders, sprays, solutions, ointments, foams, face masks, or patches. For pharmaceutical applications, the MMP inhibitor may be delivered using a patch, by injection, using a microneedle delivery system, using iontophoresis, using electroporation, or using ultrasound. The inventive skin care system reduces the proteolytic degradation of proteins in the skin that lead to older looking, less attractive, and/or less appealing skin. The inventive therapeutic skin care system may be used to treat a skin disease or disorder associated with abnormal or undesired MMP activity (e.g., cancer or other proliferative disease, inflammatory disease).

### MMP Inhibitors

The present invention provides novel matrix metalloproteinase inhibitors. The compound may be selective for one or more particular classes of MMPs, or the compound may generally inhibit MMPs. In certain embodiments, the compound inhibits MMPs found in the skin, in particular those secreted by inflammatory cells found in the skin. The activity of these compounds may be assessed by MMP assays known in the art such as those described in Puerta et al. J. Am. Chem. Soc. 126:8388-8389, 2004; Fesik et al. J. Am. Chem. Soc. 119:5818-5827, 1997; each of which is incorporated herein by reference. Kits for assessing MMP inhibitory activity may also be purchased from commercial sources such as Biomol International. In certain embodiments, the IC₅₀ of the compound in a standard assay for MMP activity is less than 10 μM, less than 1 μM, less than 0.1 μM, less than 0.01 μM or less than 0.001 μM. Generally, the compounds include a zinc binding group, which binds the zinc atom at the active site of matrix metalloproteinases. Other functional groups around the zinc binding group may provide increased binding in the active site of an MMP.

Compounds of the invention are of the formula:

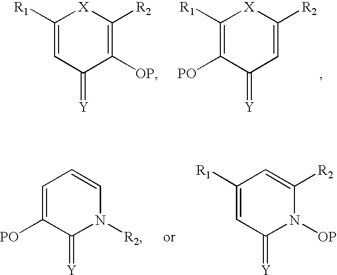

wherein

X is O or NR′, wherein R′ is hydrogen, C₁-C₆ alkyl, or a nitrogen-protecting group;

Y is O or S;

P is hydrogen or an oxygen-protecting group;

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —C(O)NHR_{A}; —CH₂NHR_{A}; —CH₂C(═O)NHR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —NO₂; —N(R_{B})₂; —NHC(O)R_{B}; —C(O)NHR_{B}; —CH₂NHR_{B}; —CH₂C(═O)NHR_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and cosmetically acceptable forms thereof.

In certain embodiments, X is O. In other embodiments, X is NH.

In certain embodiments, Y is O. In other embodiments, Y is S.

In certain embodiments, P is hydrogen. In other embodiments, P is an oxygen-protecting group. In certain embodiments, P is a silicon-containing protecting group. In certain embodiments, P is C₁-C₆ alkyl. In certain embodiments, P is acyl.

In certain embodiments, R₁ is hydrogen. In certain embodiments, R₁ is C₁-C₆ alkyl. In certain embodiments, R₁ is methyl. In certain embodiments, R₁ is ethyl. In certain embodiments, R₁ is propyl.

In certain embodiments, R₂ is an aryl-containing or heteroaryl-containing moiety, wherein the aryl or heteroaryl group is optionally substituted. In certain embodiments, R₂ is an aryl-containing moiety, wherein the aryl group is substituted. In certain embodiments, R₂ is a heteroaryl-containing moiety, wherein the heteroaryl group is substituted. In certain embodiments, R₂ is substituted or unsubstituted arylalkyl. In certain particular embodiments, R₂ is benzyl. In certain embodiments, R₂ is substituted benzyl. In certain embodiments, R₂ is substituted or unsubstituted arylcarbamyl. In certain embodiments, R₂ is substituted phenylcarbamyl. In certain embodiments, R₂ is substituted biphenylcarbamyl. In certain embodiments, R₂ is substituted phenylcarbamyl(C₁-C₆)alkyl. In certain embodiments, R₂ is substituted biphenylcarbamyl(C₁-C₆)alkyl. In certain embodiments, R₂ is substituted triphenylcarbamyl(C₁-C₆)alkyl. In certain embodiments, R₂ is substituted phenyl(C₁-C₆)alkyl carbamyl. In certain embodiments, R₂ is substituted biphenyl(C₁-C₆)alkyl carbamyl. In certain embodiments, R₂ is substituted triphenyl(C₁-C₆)alkyl carbamyl. In certain embodiments, R₂ is substituted phenyl(C₁-C₆)alkylcarbamyl(C₁-C₆)alkyl. In certain embodiments, R₂ is substituted biphenyl(C₁-C₆)alkycarbamyl(C₁-C₆)alkyl. In certain embodiments, R₂ is substituted triphenyl(C₁-C₆)alkycarbamyl(C₁-C₆)alkyl. In certain embodiments, R₂ is substituted 4-phenylbiphenylcarbamyl. In certain embodiments, R₂ is substituted 4-phenylbiphenyl(C₁-C₆)alkylcarbamyl. In certain embodiments, R₂ is substituted 4-phenylbiphenylcarbamyl(C₁-C₆)alkyl. In certain embodiments, R₂ is substituted 4-phenylbiphenyl(C₁-C₆)alkylcarbamyl(C₁-C₆)alkyl. In certain embodiments, R₂ is substituted phenoxyphenylcarbamyl. In certain embodiments, R₂ is substituted phenoxyphenylcarbamyl(C₁-C₆)alkyl. In certain embodiments, R₂ is substituted phenoxyphenyl(C₁-C₆)alkyl carbamyl(C₁-C₆)alkyl. In certain embodiments, R₂ is substituted phenylamino. In certain embodiments, R₂ is substituted phenyl(C₁-C₆)alkylamino. In certain embodiments, R₂ is substituted phenylamino(C₁-C₆)alkyl. In certain embodiments, R₂ is substituted phenyl(C₁-C₆)alkylamino(C₁-C₆)alkyl. In certain embodiments, R₂ is substituted (C₆-C₁₀)arylamino. In certain embodiments, R₂ is substituted (C₆-C₁₀)aryl(C₁-C₆)alkylamino. In certain embodiments, R₂ is substituted (C₆-C₁₀)arylamino(C₁-C₆)alkyl. In certain embodiments, R₂ is substituted (C₆-C₁₀)aryl(C₁-C₆)alkylamino(C₁-C₆)alkyl. In certain embodiments, R₂ is substituted biphenyl(C₁-C₆)alkylamino. In certain embodiments, R₂ is substituted biphenylamino(C₁-C₆)alkyl. In certain embodiments, R₂ is substituted biphenyl(C₁-C₆)alkylamino(C₁-C₆)alkyl.

In certain embodiments, R₂ is of the formula:

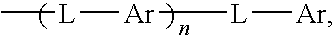

wherein

each occurrence of L is independently a covalent bond or a substituted or unsubstituted aliphatic or heteroaliphatic linker;

each occurrence of Ar is independently a substituted or unsubstituted aryl or heteroaryl ring; and

n is an integer from 0 to 6, inclusive. In certain embodiments, L is a covalent bond. In certain embodiments, L is a substituted or unsubstituted aliphatic linker. In certain embodiments, L is a substituted or unsubstituted alkylidene moiety. In certain embodiments, L is a substituted or unsubstituted alkenylidene or alkynylidene moiety. In certain embodiments, L is a substituted or unsubstituted heteroaliphatic linker. In certain embodiments, L is a covalent bond or of one of the formulae:

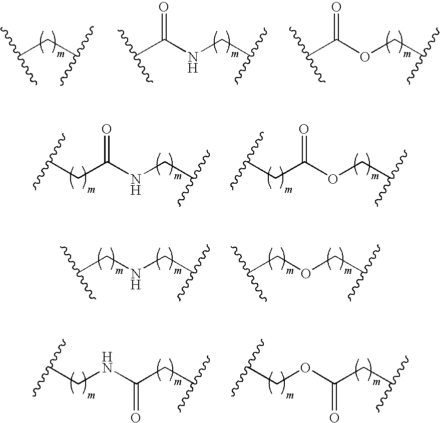

wherein each occurrence of m is an integer between 0 and 6, inclusive. In certain embodiments, at least one Ar is a substituted aryl or heteroaryl ring. In certain embodiments, at least one Ar is a substituted aryl ring. In certain embodiments, at least one Ar is a substituted phenyl ring. In certain embodiments, at least one Ar is a substituted or unsubstituted heteroaryl ring. In certain embodiments, at least one Ar is a substituted heteroaryl ring. In certain embodiments, at least one Ar is an unsubstituted heteroaryl ring. In certain embodiments, n is 0. In certain embodiments, n is 1. In certain embodiments, n is 2. In certain embodiments, n is 3. In certain embodiments, n is 4.

In certain embodiments, R₂ is substituted or unsubstituted heteroarylalkyl. In certain embodiments, R₂ is substituted or unsubstituted heteroarylcarbamyl.

In certain embodiments, R₂ is of one of the formulae:

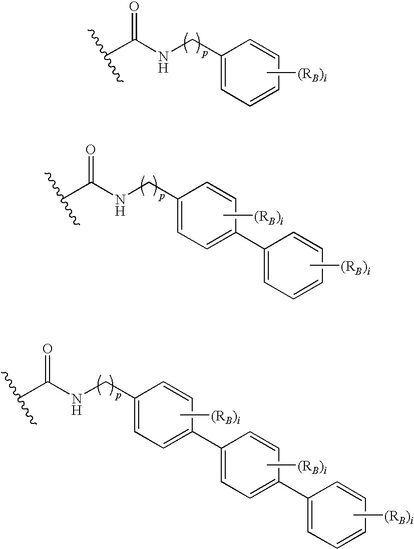

wherein

p is an integer between 0 and 6, inclusive;

each occurrence of i is independently an integer between 0 and 5, inclusive, and at least one i is at least 1; and

each occurrence R_{B} is independently halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}′; —C(═O)R_{B}′; —CO₂R_{B}′; —CN; —SCN; —SR_{B}′; —SOR_{B}′; —SO₂R_{B}′; —NO₂; —N(R_{B}′)₂; —NHC(O)R_{B}′; or —C(R_{B}′)₃; wherein each occurrence of R_{B}′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. In certain embodiments, at least one R_{B} is halogen. In certain embodiments, at least one R_{B} is C₁-C₆ alkyl. In certain embodiments, at least one R_{B} is —OH. In certain embodiments, at least one R_{B} is —NH₂. In certain embodiments, at least one R_{B} is acyl. In certain embodiments, at least one R_{B} is acetyl.

In certain embodiments, the compound is of one of the formulae:

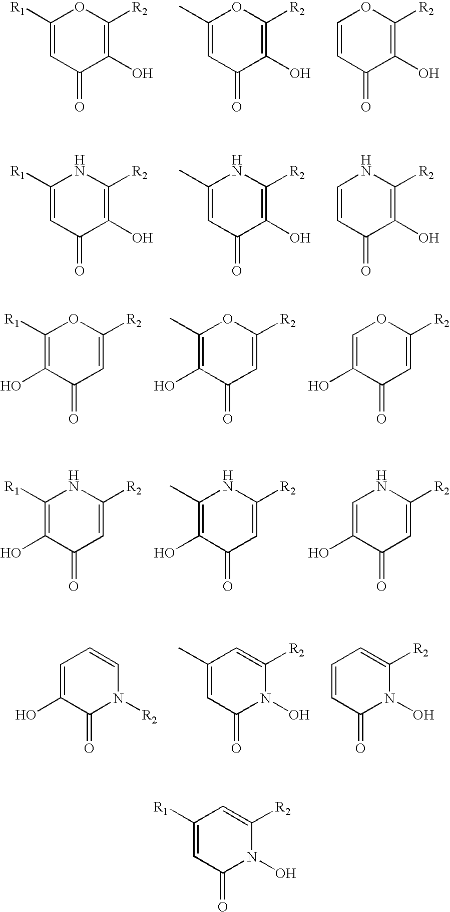

wherein R₁ and R₂ are as defined in the genera, classes, subclasses, and species described herein.

The compounds of the invention may be prepared based on synthetic methodologies described in U.S. patent applications, U.S. Ser. No. 60/566,882, filed Apr. 29, 2004; U.S. Ser. No. 60/576,444, filed Jun. 3, 2004; and U.S. Ser. No. 60/826,488, filed Sep. 21, 2006; and PCT applications, WO 05/110399, published Nov. 24, 2005; WO 06/028523, published Mar. 16, 2006; each of which is incorporated herein by reference. The compounds may be purified and characterized using any methods known in the art. In certain embodiments, the compounds is at least 90% pure, at least 95% pure, at least 99% pure, or at least 99.9% pure.

### Cosmetic Compositions

The present invention also provides cosmetic compositions comprising an MMP inhibitor and a cosmetically suitable vehicle. The cosmetic composition is formulated for application to the skin of a subject (e.g., a human). The cosmetic composition may be a cream, a lotion, a solution, an ointment, an emulsion, a powder, a spray, a gel, a paste, a serum, a solid stick, a foam, a patch, a face mask, or other composition suitable for application to the skin. In certain embodiments, the vehicle allows for the easy application of the MMP inhibitor to the skin and optionally may increase its effectiveness in reducing or preventing the signs of aging or sun damage. In certain embodiments, the vehicle allows for the easy application of the MMP inhibitor to the skin and optionally may increase its effectiveness in improving the appearance of skin. In certain embodiments, the cosmetic composition is designed to deliver the MMP inhibitor to multiple layer of the skin. In certain embodiments, the cosmetic composition is designed to deliver the MMP inhibitor to only the outermost layer of skin. In certain embodiments, the cosmetic composition is designed to deliver the MMP inhibitor to only the epidermis. In certain embodiments, the cosmetic composition is designed to deliver the MMP inhibitor to only the surface of the skin.

Without wishing to be bound by any particular theory the use of MMP inhibitors on skin is thought to create the desired effect by decreasing the degradation of collagen in the treated skin, increasing the levels of collagen and/or procollagen in the skin, preventing the decrease in levels of collagen and/or procollagen in the skin, and/or stimulating the formation of new collagen or new collagen in the skin. The levels of other extracellular matrix (ECM) proteins (e.g., elastin, fibronectin, laminins) in the skin may also be increased or maintained using MMP inhibitors. The level of degradation of procollagen, collagen, or other ECM proteins in the treated skin may be decreased by at least about 10%, at least about 20%, at least about 25%, at least about 30%, at least about 40%, at least about 50%, at least about 60%, at least about 70%, at least about 75%, at least about 80%, or at least about 90%. The formation of procollagen, collagen, or other ECM proteins in the treated skin may be increase by at least bout 5%, at least about 10%, at least about 20%, at least about 25%, at least about 30%, at least about 40%, at least about 50%, at least about 60%, at least about 70%, at least about 75%, at least about 80%, or at least about 90%. Methods of detecting the levels of procollagen, collagen, or other ECM proteins are known in the art. Any method of detecting these proteins in treated skin may be used. In certain embodiments, the method is quantitative. In certain embodiments, methods for measuring collagen content are those described in Plastow et al., “Early Changes in Dermal Collagen of Mice Exposed to Chronic UVB Irradiation and the Effects of a UVB Sunscreen” Journal of Investigative Dermatology, 91:590-92, 1998; which is incorporated herein by reference. In summary, the method of Plastow et al. uses SDS-polyacrylamide gel electrophoresis to separate proteins from digested skin samples. The results of such a separation can be used to calculate a percent decrease in collagen degradation for treated skin versus untreated skin. Immunohistochemistry may also be used to determine levels of collagen, procollagen, or other ECM proteins in the skin. For example, Kim et al. describes immunohistochemical techniques for determining procollagen levels in the skin. “Photoprotective and anti-skin-aging effects of eicosapentaenoic acid in human skin in vivo” Journal of Lipid Research 46:921-930, 2006, which is incorporated herein by reference. Radiolabeling may also be used to measure the biosynthesis of new procollagen, collagen, or other ECM proteins. For example, total collagen biosynthesis can be measured using ¹⁴C-proline incorporation. Fresh skin samples are incubated in culture media with radiolabeled proline for 1 day. Following the incubation period, the skin samples are acidified with 0.1N acetic acid and incubated with 1 mg/mL pepsin to digest the non-collagenous matrix. Samples are then lyophilized and subjected to SDS-polyacrylamide gel electrophoresis. Trayhurn et al., “The Metabolism of Amino Acids in the Bovine Lens” Biochem. J. 136:67-75, 1973; incorporated herein by reference.

Many MMP inhibitors are poorly soluble in many vehicles; therefore, an appropriate solubilizer and method of formulating the MMP inhibitor is needed to prepare a cosmetic composition with an MMP inhibitor. Useful solubilizers include ethers, alkoxylated alcohols, alkoxylated amines, sorbitan esters, phospholipids, and fatty quaternaries. The vehicle may also include emollients which lubricate or hydrate the skin. The cosmetic composition may in addition to an MMP inhibitor also include another active ingredient such as a vitamin, anti-inflammatory agent, retinoid, anti-oxidant, steroid, caffeine, sunscreen, protein, peptide, carbohydrate, lipid, polynucleotide, or other biologically active agent. The cosmetic composition may include multiple MMP inhibitors. The composition may also include a preservative, a coloring agent, optical agent, or a fragrance.

Any MMP inhibitor may be utilized in the inventive cosmetic compositions. In certain embodiments, the MMP inhibitor is described herein such as one of the compounds of the invention. In other embodiments, the MMP inhibitor is described in U.S. patent applications, U.S. Ser. No. 60/566,882, filed Apr. 29, 2004; U.S. Ser. No. 60/576,444, filed Jun. 3, 2004; and U.S. Ser. No. 60/826,488, filed Sep. 21, 2006; and PCT applications, WO 05/110399, published Nov. 24, 2005; WO 06/028523, published Mar. 16, 2006; each of which is incorporated herein by reference. Other MMP inhibitors are described in U.S. Pat. Nos. 7,094,752; 7,029,713; 6,942,870; 6,919,072; 6,906,036; 6,890,937; 6,884,425; 6,858,598; 6,759,432; 6,750,233; 6,750,228; 6,713,074; 6,699,486; 6,645,477; 6,630,516; 6,548,667; 6,541,489; 6,379,667; 6,365,630; 6,130,254; 6,093,398; 5,962,466; and 5,837,224; each of which is incorporated herein by reference. Still other MMP inhibitors are described in published U.S. patent applications 20070037253; 20060293345; 20060173183; 20060084688; 20050058709; 20050020607; 20050004177; 20040235818; 20040185127; 20040176393; 20040067883; 20040048852; 20040034098; 20040034086; 20040034085; 20040023969; 20040019055; 20040019054; 20040019053; 20040006137; 20040006077; 20030212048; 20030004165; 20020198176; 20020177588; 20020169314; 20020164319; 20020106339; 20020061866; 20020054922; 20020049237; 20020010162; 20010039287; and 20010014688; each of which is incorporated herein by reference. MMP inhibitors have also been described in the scientific literature, see, for example, Whittaker et al. Chem Rev. 99:2735-2776, 1999; which is incorporated herein by reference.

In certain embodiments, the MMP inhibitor utilized in the cosmetic composition is of one of the formulae:

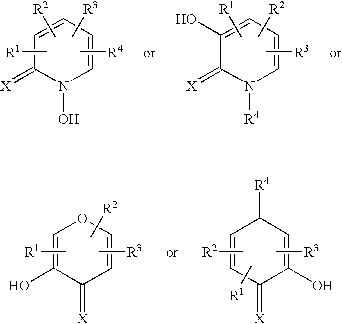

wherein

X is O or S; and

each of R¹, R², R³, and R⁴ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —C(O)NHR_{A}; —CH₂NHR_{A}; —CH₂C(═O)NHR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

and at least one of R¹, R², R³, and R⁴ is not hydrogen; or a cosmetically acceptable form thereof.

In certain embodiments, the MMP inhibitor utilized in the cosmetic composition is of one of the formulae:

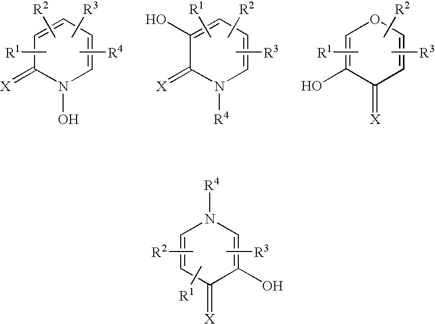

wherein X is O or S, and each R¹, R², R³, and R⁴ is individually hydrogen or another substituent, wherein at least one of R¹, R², R³, and R⁴ is an organic substituent, or a cosmetically acceptable form (e.g., salt, pro-drug, stereoisomer, etc.) thereof. In certain embodiments, at least two of R¹, R², R³, and R⁴ is an organic substituent.

In certain embodiments, at least one of R¹, R², R³, or R⁴ comprises one or more amido and/or amino moieties. In certain embodiments, at least one of R¹, R², R³, or R⁴ contains one or more peptidyl residues. In certain embodiments, at least one of R¹, R², R³, or R⁴ is a naturally-occurring peptide, a synthetic peptide, or a peptide analog (e.g., a peptidomimetic). In certain embodiments, one or two of R¹, R², R³, or R⁴ comprises one or more amino moieties (—C(O)NH—). In certain embodiments, one or two of R¹, R², R³, and R⁴ is [[(C₆-C₁₀)aryl]_{q}-[O]ₚ—[(C₆-C₁₀)aryl]-[O]ᵣ—(C₁-C₆)alkyl]ₒ-[C(O)]ₛ—[N(R)]—[C(O)]ₜ—[C₁-C₆)alkyl]_{w}-], wherein q, p, r, o, s, t, and w are individually 0 or 1, and wherein R is H, (C₁-C₄)alkyl, phenyl, or benzyl. In certain embodiments, R¹, R², R³, or R⁴ are individually biphenylcarbamyl, biphenylcarbamyl(C₁-C₆)alkyl, biphenyl(C₁-C₆)alkylcarbamyl, biphenyl(C₁-C₆)alkylcarbamyl(C₁-C₆)alkyl, phenoxyphenylcarbamyl, (C₆-C₁₀)aryl(C₁-C₆)alkylamino(C₁-C₆)alkyl, biphenyl(C₁-C₆)alkylamino(C₁-C₆)alkyl, (C₆-C₁₀)arylcarbonylamino(C₁-C₆)alkyl, (C₆-C₁₀)aryl(C₁-C₆)alkylcarbonylamino(C₁-C₆)alkyl, biphenyloxy(C₁-C₆)alkycarbonylamino(C₁-C₆)alkyl, or phenoxyphenylcarbamyl(C₁-C₆)alkyl, wherein the phenyl or aryl group(s) may be optionally substituted. In certain embodiments, R¹, R², R³, and R⁴ individually are, or are an organic substituent substituted with, at least one of H, halo, CN, nitro, amino, sulfonamido, (C₁-C₆)alkyl, (C₁-C₆)alkoxy, (C₃-C₆)cycloalkyl, (C₃-C₆)cycloalkyl((C₁-C₆)alkyl), (C₆-C₁₀)aryl, (C₆-C₁₀)aryl(C₂-C₁₀)alkyl, (C₆-C₁₀)heteroaryl, (C₃-C₆)heterocycloalkyl, (C₃-C₆)heterocycloalkyl(C₁-C₆)alkyl, (C₂-C₆)alkenyl, (C₂-C₆)alkynyl, (C₁-C₆)alkanoyl, halo(C₁-C₆)alkyl, hydroxyl(C₁-C₆)alkyl, (C₁-C₆)alkoxycarbonyl, (C₁-C₆)alkylthio, thio(C₁-C₆)alkyl, (C₁-C₆)alkanoyloxy, N(R⁶)(R⁷), or SO₂N(R⁶)(R⁷), wherein R⁶ and R⁷ are individually H, ═O, —OH, (C₁-C₆)alkyl, (C₃-C₆)cycloalkyl, (C₃-C₆)cycloalkyl(C₁-C₆)alkyl, phenyl, or benzyl, or R⁶ and R⁷, together with the N to which they are attached, form a 5- or 6-membered ring which may optionally contain 1-2 S, N(R⁶), or nonperoxide O; or R¹ and R² together are methylenedioxy, optionally any of R¹, R², R³, and R⁴ is substituted with one to four R¹; or a pharmaceutically acceptable salt thereof. In certain embodiments, one of R¹, R², R³, R⁴ is (C₁-C₃)alkyl. In certain embodiments, one of R¹, R², R³, or R⁴ is H. In certain embodiments, one of R¹, R², R³, or R⁴ comprises an NR′, moiety, wherein R′ is (C₁-C₄)alkyl, benzyl, t-Boc, (C₃-C₆)cycloalkyl(C₁-C₃)alkyl, or H. In certain embodiments, one or two of R², R³, or R⁴ comprises one or two (C₆-C₁₂) aryl groups. In certain embodiments, R¹ is halo, lower alkyl, sulfonamido, amino, —NO₂, or —CN. In certain embodiments, the organic substituent comprises the moiety —C(R⁵)(R⁸)—C(O)NH— wherein R⁵ is H, and R⁸ is (C₁-C₂₂)alkyl, (C₂-C₆)alkenyl, (C₆-C₁₀)aryl, (C₁-C₆)alkyl, (C₆-C₁₀)heteroaryl, (C₆-C₁₀)heteroaryl(C₁-C₆)aryl, (C₃-C₆)cycloalkyl, (C₃-C₆)cycloalkyl(C₁-C₆)alkyl, or R⁵ and R⁸ taken together with the carbon atom to which they are attached can be (C₄-C₆) spiroalkyl or spiroheterocycloalkyl. In certain embodiments, R¹, R², R³, or R⁴ is an organic substituent terminated by —C(O)N(R⁶)(R⁷). In certain embodiments, R¹, R², R³, or R⁴ is —C(R⁸)—C(O)N(H)—CH(R⁹)—C(O)NH(R¹⁰) or —C(R⁵)(R⁸)—C(O)N(H)—CH(R⁹)—C(O)NH(R¹⁰) wherein R⁵, R⁸, R⁹, and R¹⁰ are each independently H, (C₁-C₂₂)alkyl, (C₂-C₆)alkenyl, (C₆-C₁₀)aryl, (cl-C₆)alkyl, (C₆-C₁₀)heteroaryl, (C₆-C₁₀)heteroaryl(C₁-C₆)aryl, (C₃-C₆)cycloalkyl, or (C₃-C₆)cycloalkyl(C₁-C₆)alkyl, or R⁵ and R⁸ taken together with the carbon atom to which they are attached can be (C₄-C₆) spiroalkyl or spiroheterocycloalkyl.

In certain embodiments, the MMP inhibitor utilized in the cosmetic composition is of one of the formulae:

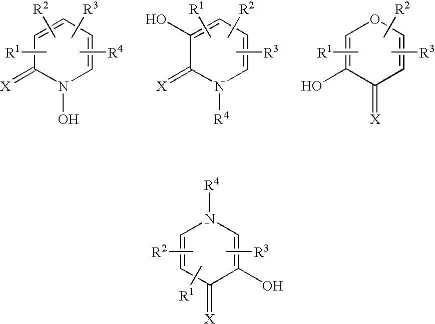

wherein X is O or S, and one or two of R¹, R², R³ and R⁴ is individually a substituent of formula: [(C₆-C₁₀)aryl]ₓ-[(C₆-C₁₀)aryl]_{q}-[O]ₚ—[(C₆-C₁₀)aryl]-[O]ᵣ—(C₁-C₆)alkyl]ₒ-[C(O)]ₛ—[N(R)]—[C(O)]ₜ—[C₁-C₆)alkyl]_{w}-], wherein q, p, r, o, s, t, w, and x are individually 0 or 1, and wherein R is H, (C₁-C₄)alkyl, phenyl, or benzyl; and the remainder of R¹, R², R³, and R⁴ are individually H, halo, CN, nitro, amino, sulfonamido, (C₁-C₆)alkyl, (C₁-C₆)alkoxy, (C₃-C₆)cycloalkyl, (C₃-C₆)cycloalkyl((C₁-C₆)alkyl), (C₆-C₁₀)aryl, (C₆-C₁₀)aryl(C₂-C₁₀)alkyl, (C₆-C₁₀)aryl(C₂-C₁₀)alkenyl, (C₆-C₁₀)heteroaryl, (C₃-C₆)heterocycloalkyl, (C₃-C₆)heterocycloalkyl(C₁-C₆)alkyl, (C₂-C₆)alkenyl, (C₂-C₆)alkynyl, (C₁-C₆)alkanoyl, halo(C₁-C₆)alkyl, hydroxyl(C₁-C₆)alkyl, (C₁-C₆)alkoxycarbonyl, (C₁-C₆)alkylthio, thio(C₁-C₆)alkyl, (C₁-C₆)alkanoyloxy, N(R⁶)(R⁷), or SO₂N(R⁶)(R⁷), wherein R⁶ and R⁷ are individually H, ═O, —OH, (C₁-C₆)alkyl, (C₃-C₆)cycloalkyl(C₃-C₆)cycloalkyl(C₁-C₆)alkyl, phenyl, or benzyl, or R⁶ and R⁷, together with the N to which they are attached, form a 5- or 6-membered ring which may optionally contain 1-2 S, N(R⁶) or nonperoxide O; or R¹ and R² together are methylenedioxy, and optionally any of R¹, R², R³, and R⁴ is substituted with one to four R¹; or a pharmaceutically acceptable salt thereof. In certain embodiments, (C₆-C₁₀)aryl is phenyl. In certain embodiments, t is 1, and w or o is 1. In certain embodiments, p is 0, s is 0, t is 1, o is 1, and w is 0. In certain embodiments, p is 0. In certain embodiments, one or two of R¹, R², R³, and R⁴ is individually biphenylcarbamyl, biphenylcarbamyl(C₁-C₆)alkyl, biphenyl(C₁-C₆)alkylcarbamyl, biphenyl(C₁-C₆)alkylcarbamyl(C₁-C₆)alkyl, 4-phenylbiphenylcarbamyl, 4-phenylbiphenyl(C₁-C₆)alkylcarbamyl, 4-(4-cyanophenyl)benzylcarbamyl, 4-methoxybenzylcarbamyl, phenoxyphenylcarbamyl, (C₆-C₁₀)aryl(C₁-C₆)alkylamino(C₁-C₆)alkyl, biphenyl(C₁-C₆)alkylamino(C₁-C₆)alkyl, (C₆-C₁₀)arylcarbonylamino(C₁-C₆)alkyl, (C₆-C₁₀)aryl(C₁-C₆)alkylcarbonylamino(C₁-C₆)alkyl, biphenyloxy(C₁-C₆)alkycarbonylamino(C₁-C₆)alkyl, or phyenoxyphenylcarbamyl(C₁-C₆)alkyl, wherein the phenyl or aryl group(s) may be optionally substituted with one to four R¹. In certain embodiments, one of R¹, R², or R³ is (C₁-C₃)alkyl. In certain embodiments, one of R₁, R₂, or R₃ is H. In certain embodiments, the inhibitor comprises an NR⁴ moiety wherein R⁴ is (C₁-C₃)alkyl, benzyl, t-Boc, (C₃-C₆)cycloalkyl(C₁-C₃)alkyl, or hydrogen. In certain embodiments, a phenyl or aryl group is substituted with (C₁-C₆)alkyl, halo, (C₁-C₆)alkoxy, heteroaryl, styryl, phenyl, CN, sulfonamide, amino, or —NO₂.

In certain embodiments, the MMP inhibitor utilized in the cosmetic composition is of the formula:

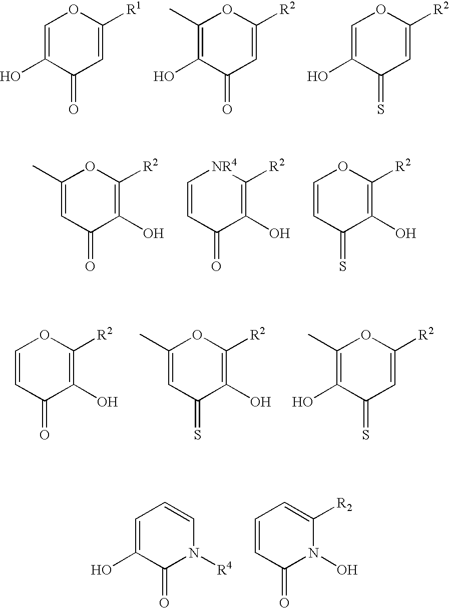

wherein

R¹, R², or R⁴ is a substituent of formula: [(C₆-C₁₀)aryl]ₓ-[(C₆-C₁₀)aryl]_{q}-[O]ₚ—[(C₆-C₁₀)aryl]-[O]ᵣ—(C₁-C₆)alkyl]ₒ-[C(O)]ₛ—[N(R)]—[C(O)]ₜ—[C₁-C₆)alkyl]_{w}-], wherein q, p, r, o, s, t, w, and x are individually 0 or 1, and wherein R is H, (C₁-C₄)alkyl, phenyl, or benzyl; and R⁴ is (C₁-C₃)alkyl, benzyl, t-Boc, (C₃-C₆)cycloalkyl(C₁-C₄)alkyl, or hydrogen. In certain embodiments, R1 or R2 is biphenylcarbamyl, biphenylcarbamyl(C₁-C₆)alkyl, biphenyl(C₁-C₆)alkylcarbamyl, biphenyl(C₁-C₆)alkylcarbamyl(C₁-C₆)alkyl, 4-phenylbiphenylcarbamyl, 4-phenylbiphenyl(C₁-C₆)alkylcarbamyl, 4-(4-cyanophenyl)benzylcarbamyl, 4-methoxybenzylcarbamyl, phenoxyphenylcarbamyl, (C₆-C₁₀)aryl(C₁-C₆)alkylamino(C₁-C₆)alkyl, biphenyl(C₁-C₆)alkylamino(C₁-C₆)alkyl, (C₆-C₁₀)arylcarbonylamino(C₁-C₆)alkyl, (C₆-C₁₀)aryl(C₁-C₆)alkylcarbonylamino(C₁-C₆)alkyl, biphenyloxy(C₁-C₆)alkycarbonylamino(C₁-C₆)alkyl, or phyenoxyphenylcarbamyl(C₁-C₆)alkyl, wherein the phenyl or aryl group(s) may be optionally substituted. In certain embodiments, R¹, R², or R⁴ is biphenylmethylcarbamyl, phenoxyphenylcarbamyl, biphenylcarbamyl, benzylaminomethyl, phenethylaminomethyl, benzoylaminomethyl, benzylcarbonylaminomethyl, phenethylcarbonylaminomethyl, phenylpropylcarbonylaminomethyl, biphenylmethylcarbamylmethyl, phenoxyphenylcarbamylmethyl, biphenylcarbamylmethyl, and biphenylyloxyethylcarbonylaminomethyl.

In certain embodiments, the MMP inhibitor utilized in the cosmetic composition is of one of the formulae:

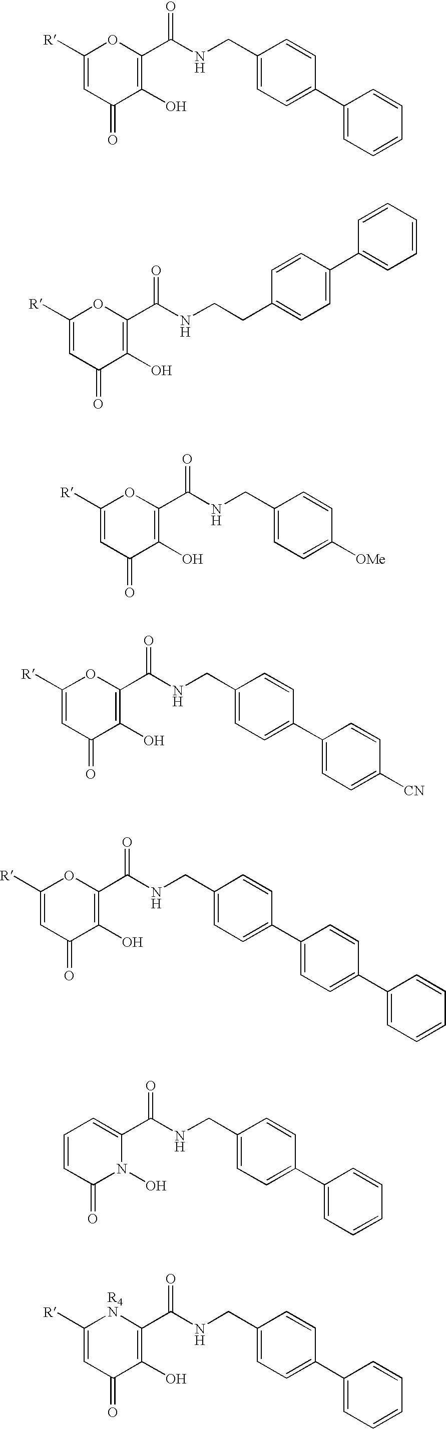

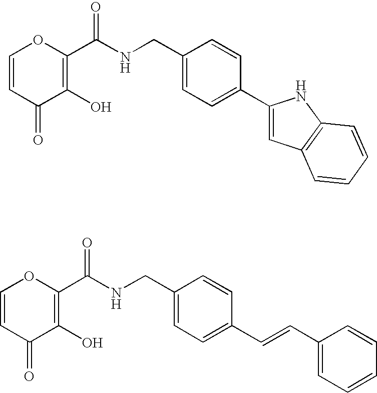

wherein R′ is H or —CH₃, and R⁴ is H, (C₁-C₃)alkyl, benzyl, t-Boc, or (C₃-C₆)cycloalkyl(C₁-C₄)alkyl. In certain embodiments, R′ is H. In other embodiments, R′ is —CH₃.

In certain embodiments, the MMP inhibitor utilized in the cosmetic composition is of one of the formulae:

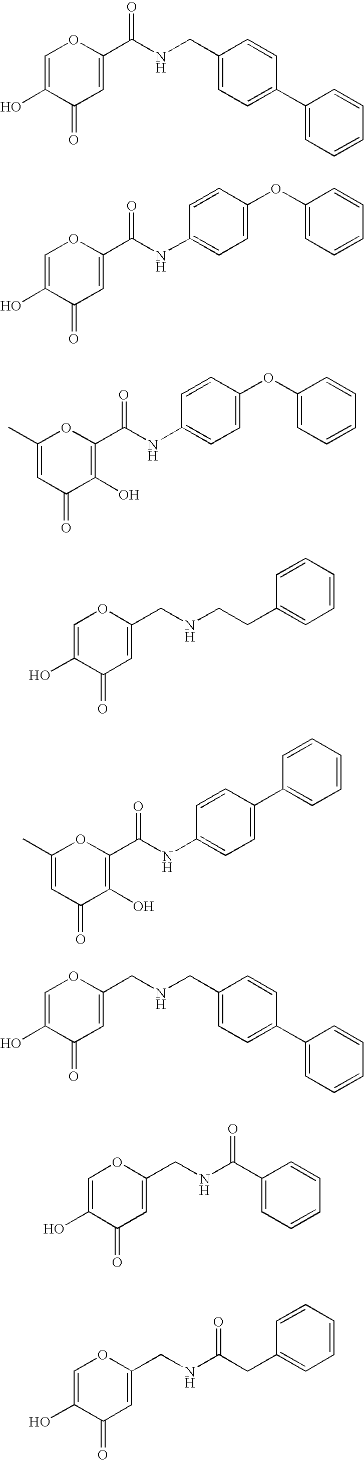

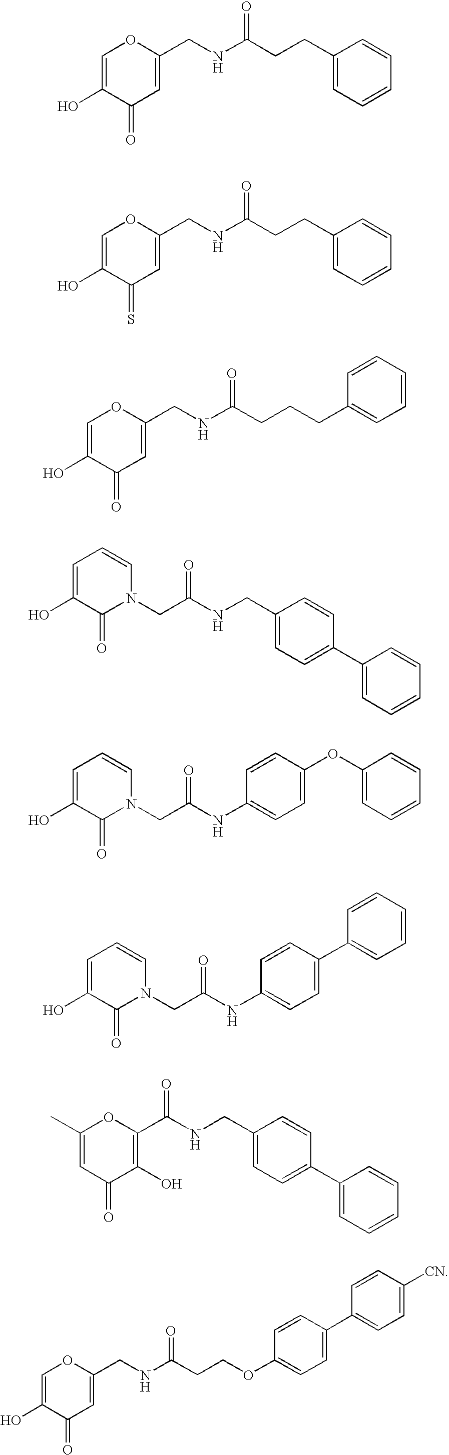

In certain embodiments, the MMP inhibitor utilized in the cosmetic composition is of the formula:

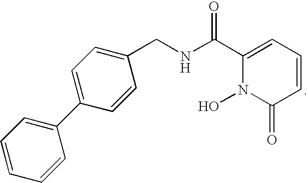

In certain embodiments, the MMP inhibitor utilized in the cosmetic composition is of the formula:

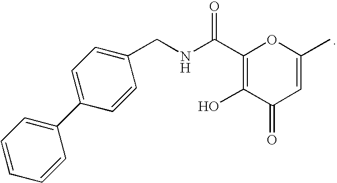

In certain embodiments, the MMP inhibitor utilized in the cosmetic composition is of the formula:

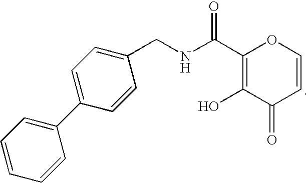

In certain embodiments, the MMP inhibitor utilized in the cosmetic composition is of the formula:

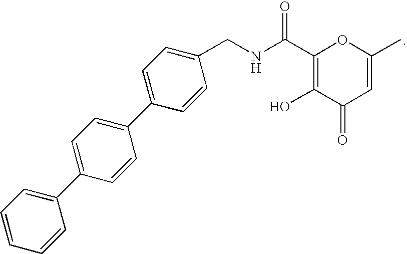

In certain embodiments, the MMP inhibitor used in the cosmetic composition is not a retinoid. In certain embodiments, the MMP inhibitor is not aspirin, E5510, a glucocorticoid, vitamin D₃, GI12947, a TIMP, a hydroxamate, a hydroxyurea derivative, or a tetracycline. In certain embodiments, the MMP inhibitor is not CGS27023A, an MMP inhibitor being developed by Novartis Pharma. In certain other embodiments, the MMP inhibitor is not genistein, galardin, batimastat, marimastat, N-acetyl cysteine, or a green tea extract.

In certain embodiments, the cosmetic composition contains a sufficiently high concentration of the MMP inhibitor such that when it is applied to skin the appearance or feel of the skin is improved. In certain embodiments, the concentration of MMP inhibitor in the composition is effective to improve the smoothness of skin. In particular, the concentration is sufficient to prevent or reduce the appearance of wrinkles. In certain embodiments, the concentration of MMP inhibitor in the composition is effective to decrease redness of the skin. In certain embodiments, the concentration of MMP inhibitor in the skin is effective to improve radiance of the skin. In certain embodiments, the concentration of MMP inhibitor in the composition is sufficient to achieve a desirable cosmetic effect (e.g., reduce the appearance of lines and wrinkles, increase radiance of the skin, make skin feel smoother, reduce hyperpigmentation, improve the look of skin, improve the feel of skin, increase firmness, increase skin tone, reduce redness, etc.).

The amount of MMP inhibitor in the composition may range from approximately 0.001% to approximately 50% by weight of the composition. In certain embodiments, the amount of MMP inhibitor is between approximately 0.01% and approximately 20%. In certain embodiments, the amount of MMP inhibitor is between approximately 0.01% and approximately 1% by weight. In certain embodiments, the amount of MMP inhibitor is between approximately 0.001% and approximately 0.1% by weight. In certain embodiments, the amount of MMP inhibitor is between approximately 0.5% and approximately 10% by weight. In certain embodiments, the amount of MMP inhibitor is between approximately 1% and approximately 5% by weight. In certain embodiments, the amount of MMP inhibitor in the composition is approximately 0.001%, 0.005%, 0.01%, 0.05%, 0.1%, 0.25%, 0.5%, 0.75%, 1%, 2%, 3%, 4%, 5%, 6%, 7%, 8%, 9%, or 10%. In certain embodiments, the amount of MMP inhibitor in the composition is approximately 0.2%, 0.3%, 0.4%, 0.5%, 0.6%, 0.7%, 0.8%, or 0.9%. In certain embodiments, the amount of MMP inhibitor in the composition is approximately 0.09%, 0.08%, 0.07%, 0.06%, 0.05%, 0.04%, 0.03%, or 0.02%. In certain particular embodiments, the amount of MMP inhibitor in the composition is approximately 0.1%. The concentration of the MMP inhibitor in the composition is typically high enough to achieve a concentration in the skin greater than the IC₅₀ value for the inhibitor. In certain embodiments, the concentrations achieved in the skin are at least approximately 2-fold, 3-fold, 5-fold, 10-fold, 50-fold, or 100-fold greater than the IC₅₀ value of the inhibitor.

The MMP inhibitor may be included in the cosmetic composition as part of a micelle, liposome, droplet, particle, microparticle, or nanoparticle. Such delivery devices may provide for timed release or extended release of the MMP inhibitor. Techniques are known in the art for preparing such delivery devices. The size and composition of these delivery devices may vary depending on the desired characteristics of the devices. In certain embodiments, the MMP inhibitor is contained within a micelle or liposome. In certain embodiments, the MMP inhibitor is contained within a particle. In particular, the MMP inhibitor is contained within a polymeric particle. The MMP inhibitor may be provided in an oil-in-water emulsion or a water-in-oil emulsion. The MMP inhibitor may be provided in a nanoemulsion.

The remainder of the composition besides the MMP inhibitor is typically a cosmetically suitable vehicle or combination of vehicles. The vehicle may solubilize the MMP inhibitor for ease of application to the skin. Certain MMP inhibitors are rather difficult to solubilize thereby benefiting from the use of a solubilizer. Generally, appropriate solubilizers includes ethers, alkoxylated alcohols, alkoxylated amines, sorbitan esters, phospholipids, and fatty quaternaries. Exemplary ethers include, but are not limited to, dimethyl isosorbide, ethoxydiglycol, and polyethylene glycol (e.g., polyethylene glycol 200, 300, 400). Exemplary amines include, but are not limited to, aminomethyl propanol, triethanolamine, and diisopropylethylamine. Exemplary sorbitan esters include, but are not limited to, polysorbate 20 (TWEEN 20) and polysorbate 80 (TWEEN 80). Exemplary alkoxylated alcohols include, but are not limited to, laureth-4, oleth-2, PEG-30 sorbitol, PEG-6 caprylic/capric glycerides, glycereth-18 ethylhexanoate, PEG-7 glyceryl cocoate, glycereth-26, glycereth-7 benzoate, methyl gluceth-20 benzoate, and dimethicone PEG-8 benzoate. Exemplary phospholipids include, but are not limited to, PHOSAL 50 PG (lecithin, propylene glycol, ethanol) and PHOSAL 75A (lecithin, alcohol, safflower oil, glyceryl stearate, coconut oil, and ascorbyl palmitate). Exemplary fatty quaternaries include, but are not limited to, linoleamidopropyl PG-dimonium chloride phosphate and myristamidopropyl PG-dimonium chloride phosphate. Other solubilizers include dimethicone, cyclopentacyloxane, PEG-12 dimethicone, phenyltrimethicone, almond oil, avocado oil, joboba oil, raspberry seed oil, squalane, isohexadecane, isooctane, isododecane, hydrogenated didecene, ethoxydiglycol oleate, caprylic/capric triglyceride, propylene glycol dicaprylate/dicaprate, isocetyl stearate, octyldodecyl octyldodecanoate, propylene glycol dipelargonate, diisopropyl adipate, triisostearin, tridecyl trimellitate, neopentyl glycol, C₁₂₋₁₅ alkyl benzoate, polyglyceryl-10 decaoleate, polyglyceryl-3 oleate, poloxamer 192 dibenzoate, PEG-8 dilaurate, PEG/PPG-18/4 co-polymer, PPG-12-buteth-16, PPG-33 butyl ether, PPG-3 benzyl ether myristate, di-PPG-3 myristyl ether adipate, PPG-15 stearyl ether, sorbitan oleate, sorbitan triolate, methyl perfluorobutyl ether, methyl perfluoroisobutyl ether, ethyl perfluorobutyl ether, ethyl perfluoroisobutyl ether, poly vinyl pyrrolidone, and butylphthalimide/isopropylphthalimide. In certain embodiments, the solubilizer is poly vinyl pyrrolidone (PVP). In certain embodiments, the solubilizer is dimethyl isosorbide. In certain embodiments, the solubilizer is polyethylene glycol. Without wishing to be bound by any particular theory, it is thought that the solubilizer does not exclude the hydrophobic MMP inhibitor from interacting with the alkoxylated moieties of the solubilizer. In certain embodiments, the MMP inhibitor is stabilized through the use of an antioxidant (e.g., BHT, vitamin C or derivatives thereof, vitamin E or derivatives thereof).

The vehicle may also function to assist in the delivery or penetration of the MMP inhibitor into the skin. For example, the cosmetic composition may include a film-forming agent to enhance the effect of the MMP inhibitor. Additionally, the vehicles may aid in lubricating or moisturizing the skin. Other active ingredients may also be included in the inventive compositions (e.g., sunscreen (derivatives of PABA, cinnamates, salicylates, etc.), steroids, retinoids, anti-inflammatory agents, vitamins (vitamin A, vitamin E, biotin, vitamin C, vitamin B₃, vitamin F, D-panthenol, etc.), antibiotics, etc.), antioxidants, proteins, peptides, polynucleotides, carbohydrates, and other bioactive agents. In certain embodiments, the compositions further comprise a plant extract (e.g., St. John's wort extract, witch hazel extract, chamomile extract, arnica extract, ginseng extract, aloe vera, green tea extract, white tea extract, etc.), coloring agent (e.g., natural and artificial pigments), fragrance, protein (e.g., tropoelastin, collagen, elastin, procollagen, fibronectin, etc.), peptide, polynucleotide, etc.

Various non-toxic, dermatologically acceptable vehicles in which MMP inhibitors are stable are available in the art. In general, lubricating vehicles which help hydrate the skin are preferred. Various cosmetically acceptable vehicles are described in U.S. Pat. Nos. 7,118,735; 7,083,780; 7,067,140; 7,001,604; 6,979,452; 6,919,072; 6,864,274; 6,790,434; 6,759,052; 6,682,749; 6,630,516; 6,451,339; 6,261,603; 6,238,284; 6,146,650; 5,922,331; 5,837,224; 5,747,051; 5,322,685; 5,254,331; 5,153,230; 4,877,805; 4,801,586; and 4,228,162; each of which is incorporated herein by reference. Cosmetically acceptable vehicles are also described in the following international and foreign patent references: WO2005/097068; WO 2004/016289; WO 89/04179; DE 3442402; EP-A-131927; GB 2139496; GB 2146525; E-A 120262; DD 217989; JP-A-60-64418; each of which is incorporated herein by reference. Any of the vehicles described herein or in the cited references may be combined to form mixtures that act as the vehicle in the inventive compositions.

In certain embodiments, the composition comprises an emulsifier as part of the cosmetically suitable vehicle. The emulsifier may be an anionic, cationic, or neutral emulsifier. In certain embodiments, the emulsifier is an anionic emulsifier selected from the group consisting of alkyl sulphate, aralkyl sulphates, alkyl ethoxy ether sulphates, alkaryl sulphonates, alkyl succinates, alkyl sulphosuccinates, N-alkoyl sarconsinates, isethionates, N-acyl taurate, sodium lauryl sulfate, sodium laureth sulfate, sodium oleyl succinate, sodium dodecylbenzenesulfonate, and sodium lauryl sarconsinate. Exemplary non-ionic or neutral emulsifiers include sorbitan ester, ethoxylated sorbitan ester, ethoxylated alkyl ether, ethoxylated fatty acid ether, fatty alcohol, ethoxylated fatty alcohol, and esters of glycerin and fatty acids. In certain embodiments, the emulsifiers are synthetic or natural polymers. In certain embodiments, the emulsifier includes silicon. In certain embodiments, the emulsifier is a silicone (e.g. dimethicone, phenyltrimethicone, PEG dimethicone, PPG dimethicone, etc.).

In certain embodiments, the composition comprises an oil, lipid, wax, fatty alcohols, glycerides, or fatty acid as part of the cosmetically suitable vehicle. Exemplary oils that may be used in the composition include triglycerides, diglycerides, monoglycerides, cholesterol, lanosterol, lanolin oil, cetyl alcohol, stearyl alcohol, cetyl ester wax, cod liver oil, soybean oil, fish liver oil, squalene, liquid paraffin, ceresin oil, 2-octyldodecanol, 2-hexyldecanol, crotamiton, 1-menthol, mentha oil, benzyl alcohol, silicone oil, white petrolatum, corn oil, avocado oil, sesame oil, etc. In certain embodiments, the composition comprises a fatty acid selected from the group consisting of salts and esters of palmitate, salts and esters of stearate, salts and esters of laurate, salts and esters of oleate, isopropyl myristate, isopropyl palmitate, cis-oleic acid, diisopropyl sebacate, diethyl sebacate, diisopropyl adipate, glycerol caprate, linoleic acid, γ-linolenic acid, homo-γ-linolenic acid, columbinic acid, eicosa-n-6,9,13)-trienoic acid, arachidonic acid, γ-linolenic acid, timnodonic acid, hexaenoic acid, sorbitan sesquioleate, polyoxyl 40 stearate, glycerol caprylate, myristyl myrisate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate, and erucyl erucate. In certain embodiments, the lipid is a naturally occurring lipid. In certain embodiments, the lipid is a phospholipid. In certain embodiments, the lipid is a glycosphingolipid. Exemplary waxes include beeswax, carnauba wax, candelilia wax, ouricuri wax, Japan wax, esparto grass wax, shellac wax, spermaceti, lanolin (wool wax), petrolatum, uropygial grease, guaruma wax, cork fibre wax, sugarcane wax, rice wax, montan wax, paraffin, lignite wax, microcrystalline wax, ceresin, ozokerite, polyethylene wax, Fischer-Tropsch waxes, octacosanyl stearate, glycerides, silicone waxes, and poly(di)methylsiloxane esters. Exemplary alcohols include lauryl alcohol, coconut fatty alcohol, myristyl alcohol, cetyl alcohol, ceteraryl alcohol, stearyl alcohol, isostearyl alcohol, oleyl alcohol, elaidyl alcohol, petroselinyl alcohol, linolyl alcohol, and linolenyl alcohol.

In certain embodiments, the composition comprises a carbohydrate as part of the cosmetically suitable vehicle. Exemplary carbohydrates include monosaccharides, disaccharides, oligosaccharides, and polysaccharides. Exemplary polysaccharides include cellulose, methylcellulose, hydroxypropylmethylcellulose, chitin, galactoarabinan, polygalactose, and polyarabinose. Exemplary glycerides includes hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid monoglyceride, malic acid diglyceride, and mixture thereof.

In certain embodiments, the composition comprises a polymer or thickening agent. The polymer may be a natural or synthetic polymer. Natural polymers include polysaccharides, nucleic acid, and proteins. Synthetic polymers include polyesters, polyureas, polycarbonates, polyvinyl alcohol, polyamides, polyethers, polyesters, polyamines, polytyrosines, polyanhydrides, polyphosphazenes, polyacrylamides, polyacrylates, polymethacrylates, polyvinylpyrrolidone, etc. Exemplary thickening agents include alginate derivatives, preneutralized carbomer 430, hydrophilic silicas, polysaccharides, xanthan gum, guar guar, agar agar, carboxymethylcellulose, hydroxyethylcellulose, polyacrylates, polyacrylamides, polyvinylpyrrolidone, and salts.

In certain embodiments, the cosmetically suitable vehicle includes a solvent. In certain embodiments, the solvent comprises water. In certain embodiments, the solvent comprises an alcohol (e.g., methanol, ethanol, isopropanol, butanol, tert-butanol, etc.). In certain embodiments, the solvent is an ether. In certain embodiments, the solvent comprises propylene glycol, butylene glycol, butylated hydroxytoluene, or glycerin. In certain embodiments, the solvent is dimethylisosorbide. In certain embodiments, the solvent is 3,6-dimethoxyfuro[3,2-b]furan. In certain embodiments, the solvent is propylene glycol. In certain embodiments, the solvent is polyethylene glycol.

In certain embodiments, the composition further comprises a preservative. In certain embodiments, the preservative is quaternium-15, methylparaben, propylparaben, or diazolidinyl urea. In certain embodiments, the preservative is a metal chelating agent. The metal chelating agent binds metal ions that might accelerate the degradation of composition. In certain embodiments, the chelating agents is EDTA (e.g., disodium EDTA, tetrasodium EDTA, or other salts of EDTA), citric acid or a salt thereof, tartaric acid or a salt thereof, organo aminophosphonic acid (e.g., tri(methylene phosphonic acid), diethylene triamine penta(methylene phosphonic acid), hexamethylene diamine tetra(methylene phosphonic acid), etc.), organo phosphonic acids, nitrilotriacetic acid, polyaminocarboxylic acids (e.g., ethylenetriamine pentacetic acid), and iminodiacetic acids (e.g., 2-hydroxyl diacetic acid, glycerol imino diacetic acid). In certain embodiments, the preservative is an anti-oxidant such as butylated hydroxytoluene (BHT), vitamin E, derivatives of vitamin E, vitamin C, derivatives of vitamin C, and sodium metabisulfite. In certain embodiments, the preservative is GERMAZIDE PMP (phenoxyethanol, chlorphenesin, methylparaben, propylparaben). Various combinations of the preservatives described herein may also be used in the inventive compositions.

Cosmetically acceptable excipients and vehicles used in the skin care industry can be broken down into several categories. Components from a category may be included or excluded from the final skin care composition depending on the use and form of the final composition (e.g., lotion, cream, spray, solid stick, powder, liquid, solution, gel, serum, face mask). The categories of excipients include: (1) preservatives/antioxidants/chelating agents; (2) sunscreen agents; (3) vitamins; (4) dyes/coloring agents; (4) proteins/amino acids; (5) plant extracts; (6) humectants; (7) fragrances/perfumes; (8) oils/emollients/lubricants/butters; (9) penetrants; (10) thickeners/viscosity modifiers; (11) polymers/resins/film formers; (12) surfactants/detergents/emulsifiers/opacifying agents; (13) volatiles/propellants/solvents; (14) liquid vehicles/solvents; (15) salts; (16) pH adjusting agents/buffers/neutralizing agents; and (17) absorbents.

### Preservatives/Antioxidants/Chelating Agents

The inventive cosmetic skin care compositions may include preservatives, antioxidants, and/or chelating agents to extend the shelf-life and/or prevent the degradation of the components of the inventive composition. Antioxidants used in skin care compositions include reducing agents and/or free radical scavengers. Exemplary preservative, antioxidants, and chelating agents useful in the inventive hair care compositions include vitamin C (ascorbic acid), glutathione, lipoic acid, uric acid, carotenes (e.g., beta-carotene), alpha-tocopherol (vitamin E), ubiquinol (coenzyme Q), melatonin, ethylenediamine tetraacetic acid (EDTA) and salts thereof, citric acid and salts thereof, EGTA, aminotrimethylene phosphonic acid, resveratrol, flavonoids, lycophene, propyl gallate, tertiary butylhydroquinone, butylated hydroxyanisole, butylated hydroxytoluene, benzoates, nitrites, nitrates, sulfites, calcium propionate, sodium bisulfite, potassium hydrogen sulfite, benzyl alcohol, methyl paraben, propyl paraben, imidazolidinylurea, sulfur dioxide, and combinations thereof. Exemplary antioxidants include Acer Palmatum Leaf Extract, Acetamidocaproic Acid, Acetyl Benzoyloxy Prasterone, Acetyl Cysteine, Agrimonia Eupatoria Root Extract, Aminoethanesulfinic Acid, Aminopropyl Ascorbyl Phosphate, Aminopropyl Tocopheryl Phosphate, Anserine, Arbutin, Alpha-Arbutin, Argon, Ascorbic Acid, Ascorbic Acid Polypeptide, Ascorbyl Dipalmitate, Ascorbyl Glucoside, Ascorbyl Methylsilanol Pectinate, Ascorbyl Palmitate, Ascorbyl Stearate, Ascorbyl Tetraisopalmitate, Ascorbyl Tocopheryl Maleate, Asiaticoside, Avena Sativa (Oat) Kernel Extract, Bacillus/Rice Bran Extract/Soybean Extract Ferment Filtrate, butylated hydroxyanisole (BHA), butylated hydroxytoluene (BHT), Bis-demethoxycurcumin, Bis-Hydroxyethyl Tocopherylsuccinoylamido, Hydroxypropane, Butylated Xylenol, 4-Butylresorcinol, Caffeic Acid, Calcium Ascorbate, Calophyllum Inophyllum Seed Oil, Camellia Sinensis Catechins, Camellia Sinensis Leaf Extract, Camellia Sinensis Leaf Oil, Carnosic Acid, Carotenoids, Chitosan Ascorbate, Chitosan Glycolate, Chitosan Salicylate, Chlorogenic Acids, Cimicifuga Dahurica Root Extract, Citrus Medica Vulgaris Fruit Extract, Coptis Chinensis Root Extract, Crotonaldehyde, Curcumin, Cyamopsis Tetragonoloba (Guar) Symbiosome Extract, Cysteine, Cysteine HCI, Decyl Mercaptomethylimidazole, Demethoxycurcumin, Diamylhydroquinone, Di-t-Butylhydroquinone, Dicetyl Thiodipropionate, Dicyclopentadiene/t-Butylcresol Copolymer, Digalloyl Trioleate, Dilauryl Thiodipropionate, Dimethoxy Di-p-Cresol, Dimethylmethoxy Chromanol, Dimyristyl Thiodipropionate, Dioleyl Tocopheryl Methylsilanol, Diosmine, Disodium Ascorbyl Sulfate, Disodium Rutinyl Disulfate, Distearyl Thiodipropionate, Ditridecyl Thiodipropionate, Dodecyl Gallate, Dunaliella Bardawil Powder, Ellagic Acid, Epigallocatechin Gallate, Epimedium Sagittatum Extract, Ergothioneine, Eriobotrya Japonica Leaf Protoplasts, Erythorbic Acid, Ethylbisiminomethylguaiacol Manganese Chloride, Ethyl Ferulate, Ethylhexyl Ferulate, Ethylhexyl Gallate, Ferulic Acid, Foeniculum Vulgare (Fennel) Seed Extract, Forsythia Suspensa Fruit Extract, Fragilaria Pinnata Extract, Furfuryl Palmitate, Glucosylrutin, Glycine Max (Soybean) Symbiosome Extract, Glycine Soja (Soybean) Oil, Glycyrrhiza Glabra (Licorice) Root Extract, Grifola Frondosa Fruit Body Extract, Haematococcus Pluvialis Extract, Haematococcus Pluvialis Oil, Haematococcus Pluvialis Powder, Hesperetin Laurate, Hesperidin Methyl Chalcone, Hydrolyzed Gardenia Florida Extract, Hydroquinone, p-Hydroxyanisole, Hydroxydecyl Ubiquinone, Hydroxylamine HCI, Hydroxylamine Sulfate, Isooctyl Thioglycolate, Isoquercitrin, Kojic Acid, Kojyl Glucoside, Kojyl Methylenedioxycinnamate, Kou-Cha Ekisu, Lactobacillus/Rice Bran/Saccharomyces/Camellia Sinensis Leaf Extract Ferment, Lactobacillus/Wasabia Japonica Root Ferment Extract, Lens Culinaris (Lentil) Symbiosome Extract, Ligusticum Striatum Root Extract, Lupinus Subcarnosus Symbiosome Extract, Lycium Chinense Fruit Extract, Lycopene, Madecassicoside, Magnesium Ascorbate, Magnesium Ascorbate/PCA, Magnesium Ascorbyl Phosphate, Malachite Extract, Malpighia Punicifolia (Acerola) Fruit Extract, Manganese Dioxide, Medicago Sativa (Alfalfa) Symbiosome Extract, Melaleuca Alternifolia (Tea Tree) Leaf Oil, Melatonin, Melia Azadirachta Conditioned Media/Culture, Methoxy PEG-7 Ascorbic Acid, Methoxy-PEG-7 Rutinyl Succinate, Methoxytrimethylphenyl Dihydroxyphenyl, Propanol, Methylene Di-t-Butylcresol, Methylsilanol Ascorbate, Monascus/Rice Ferment, Murraya Exotica Leaf Extract, Nordihydroguaiaretic Acid, Octadecyl Di-t-butyl-4-hydroxyhydrocinnamate, Octanicotinoyl Epigallocatechin Gallate, Olea Europaea (Olive) Bud Extract, Olea Europaea (Olive) Fruit Unsaponifiables, Oxycoccus Palustris Seed Oil, Palmitoyl Camellia Sinensis Extract, Palmitoyl Grape Seed Extract, PEG/PPG-2/5 Tocopheryl Ether, PEG/PPG-5/10 Tocopheryl Ether, PEG/PPG-5/20 Tocopheryl Ether, PEG/PPG-5/30 Tocopheryl Ether, PEG/PPG-30/10 Tocopheryl Ether, PEG/PPG-50/20 Tocopheryl Ether, PEG/PPG-70/30 Tocopheryl Ether, PEG/PPG-100/70 Tocopheryl Ether, Pentaerythrityl Tetra-di-t-butyl Hydroxyhydrocinnamate, Perilla Ocymoides Seed Extract, Phenylthioglycolic Acid, Phloroglucinol, Pikea Robusta Extract, Pinus Pinaster Bark Extract, Pinus Radiata Bark Extract, Piper Nigrum (Black Pepper) Seed, Pisum Sativum Symbiosome Extract, Platycodon Grandiflorum Root Extract, Polygonum Cuspidatum Root Extract, Potassium Ascorbyl Tocopheryl Phosphate, Potassium Sulfite, PPG-2 Tocophereth-5, PPG-5 Tocophereth-2, PPG-10 Tocophereth-30, PPG-20 Tocophereth-50, PPG-30 Tocophereth-70, PPG-70 Tocophereth-100, PPG-5 Tocopheryl Ether, Propyl Gallate, Prunus Cerasus (Bitter Cherry) Extract, Pyridoxine Serinate, Quercetin, Quercetin Caprylate, Resveratrol, Rhodochrosite Extract, Rosmarinic Acid, Rosmarinus Officinalis (Rosemary) Flower Extract, Rosmarinus Officinalis (Rosemary) Leaf Extract, Rutin, Ryoku-Cha Ekisu, Salnacedin, Sargassum Pallidum Extract, Sarothamnus Scoparius Extract, Sedum Rosea Root Extract, Smithsonite Extract, Sodium Ascorbate, Sodium Ascorbyl/Cholesteryl Phosphate, Sodium Ascorbyl Phosphate, Sodium Bisulfite, Sodium Erythorbate, Sodium Metabisulfite, Sodium Phosphono-Pyridoxylidenerhodanine, Sodium Sulfite, Sodium Thioglycolate, Sodium Tocopheryl Phosphate, Sorbityl Furfural, TBHQ, Tetrabutyl Ethylidinebisphenol, Tetradibutyl Pentaerithrityl Hydroxyhydrocinnamate, Tetrahexyldecyl Ascorbate, Tetrahydrobisdemethoxydiferuloylmethane, Tetrahydrodemethoxydiferuloylmethane, Tetrahydrodiferuloylmethane, Tetramethylchromanol Glucosides, Thioctic Acid, Thiodiglycol, Thiodiglycolamide, Thiodiglycolic Acid, Thioglycolic Acid, Thiolactic Acid, Thiosalicylic Acid, Thiotaurine, Tococysteamide, Tocophereth-5, Tocophereth-10, Tocophereth-12, Tocophereth-18, Tocophereth-50, Tocopherol, Tocophersolan, Tocopheryl Acetate, Tocopheryt Linoleate, Tocopheryl Linoleate/Oleate, Tocopheryl Nicotinate, Tocopheryl Succinate, Tocoquinone, Toluene, o-Tolyl Biguanide, Totarol, Tremella Fuciformis Extract, Trifolium Pratense (Clover) Symbiosome Extract, Trigonella Foenum Symbiosome Extract, Tripropylene Glycol, Tris(Nonylphenyl) Phosphite, Trisodium Fructose Diphosphate, Ubiquinone, Uuron-Cha Ekisu, Vaccinium Myrtillus Bud Extract, Vaccinium Myrtillus Stem Extract, Vaccinium Vitis-Idaea Leaf Protoplasts, Vaccinium Vitis-Idaea Seed Oil, Vicia Sativa Symbiosome Extract, Vitis Vinifera (Grape) Seed Extract, Xylyl Dibutylbenzofuranone, and Zinc Dibutyldithiocarbamate. Exemplary chelating agents useful in accordance with the present invention include Acrylic Acid/Acrylamidomethyl Propane Sulfonic Acid Copolymer, Beta-Alanine Diacetic Acid, Aminotrimethylene Phosphonic Acid, Calcium Disodium EDTA, Citric Acid, Citrus Medica Vulgaris Fruit Extract, Cyclodextrin, Cyclohexanediamine Tetraacetic Acid, Diammonium Citrate, Diammonium EDTA, Diethylenetriamine Pentamethylene Phosphonic Acid, Dipotassium EDTA, Disodium Azacycloheptane Diphosphonate, Disodium EDTA, Disodium Pyrophosphate, EDTA, Etidronic Acid, Galactaric Acid, Galacturonic Acid, Alpha-Glucan, Gluconic Acid, Glucuronic Acid, HEDTA, Humic Acids, Hydroxypropyl Cyclodextrin, Lauroyl Ethylenediamine Triacetic Acid, Methyl Cyclodextrin, Methyl Dihydroxybenzoate, Oxyquinoline, Oxyquinoline Sulfate, Pentapotassium Triphosphate, Pentasodium Aminotrimethylene Phosphonate, Pentasodium Ethylenediamine Tetramethylene Phosphonate, Pentasodium Pentetate, Pentasodium Triphosphate, Pentetic Acid, Phosphonobutanetricarboxylic Acid, Phytic Acid, Potassium Citrate, Potassium EDTMP, Potassium Gluconate, Potassium Polyphosphate, Potassium Trisphosphonomethylamine Oxide, Ribonic Acid, Sodium Chitosan Methylene Phosphonate, Sodium Citrate, Sodium Diethylenetriamine Pentamethylene Phosphonate, Sodium Dihydroxyethylglycinate, Sodium EDTMP, Sodium Gluceptate, Sodium Gluconate, Sodium Glycereth-1 Polyphosphate, Sodium Hexametaphosphate, Sodium Lauroyl Ethylenediamine Triacetate, Sodium Metaphosphate, Sodium Metasilicate, Sodium Phytate, Sodium Polydimethylglycinophenolsulfonate, Sodium Polyphosphate, Sodium Trimetaphosphate, TEA-Cocamide Diacetate, TEA-EDTA, TEA-Polyphosphate, Tetrahydroxyethyl Ethylenediamine, Tetrahydroxypropyl Ethylenediamine, Tetrahydroxypropyl Ethylenediamine Dioleate, Tetrapotassium Etidronate, Tetrapotassium Pyrophosphate, Tetrasodium Dicarboxymethyl Aspartate, Tetrasodium EDTA, Tetrasodium Etidronate, Tetrasodium Glutamate Diacetate, Tetrasodium Iminodisuccinate, Tetrasodium Pyrophosphate, Tripotassium EDTA, Trisodium Dicarboxymethyl Alaninate, Trisodium EDTA, Trisodium Ethylenediamine Disuccinate, Trisodium Fructose Diphosphate, Trisodium HEDTA, Trisodium NTA, and Trisodium Phosphate. In certain embodiments, the preservative is ascorbic acid. In certain embodiments, the preservative is butylated hydroxytoluene (BHT). In certain embodiments, the preservative is tocopherol acetate. In certain embodiments, the preservative is a combination of butylated hydroxytoluene (BHT), ascorbic acid or a derivative thereof (e.g., vitamin C palmitate), and tocopherol or a derivative thereof. In certain embodiments, the preservative is Germazide PMP. The inventive composition may include 0% to approximately 4% by weight of the preservative, antioxidant, or chelating agent. In certain embodiments, the composition includes approximately 0.0001% to approximately 5% by weight of the preservative, antioxidant, or chelating agent. In certain embodiments, the composition includes approximately 0.0005% to approximately 3% by weight of the preservative, antioxidant, or chelating agent. In certain embodiments, the composition includes approximately 0.001% to approximately 2% by weight of the preservative, antioxidant, or chelating agent. In certain embodiments, the composition includes approximately 0.1% to approximately 1% by weight of the preservative, antioxidant, or chelating agent.

### Sunscreen Agents

The inventive cosmetic skin care compositions may include a sunscreen agent to protect the treated skin from the damaging ultraviolet rays of the sun. In certain embodiments, the sunscreen agent protects the treated skin from damaging UV-A and/or UV-B rays. In certain embodiments, the sunscreen agent absorb light in the wavelength range from approximately 150 nm to approximately 400 nm. In certain embodiments, the sunscreen agent absorb light in the wavelength range from approximately 250 nm to approximately 390 nm. Exemplary sunscreen agents useful in the inventive skin care compositions include p-aminobenzoic acid (PABA), PABA-derivatives (e.g., allantoin PABA, butyl PABA, dimethyl PABA ethyl cetearyldimonium tosylate, ethyl dihydroxypropyl PABA, ethylhexyl dimethyl PABA, N-ethyl-3-nitro PABA, ethyl PABA, glyceryl PABA, PEG-25 PABA, pentyl dimethyl PABA, and triPABA panthenol), avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, zinc oxide, ecamsule, terephthalylidene dicamphor sulfonic acid, 4-methylbenzylidene camphor, bisoctrizole, methylene bis-benzotriazolyl tetramethylbutylphenol, bemotrizinol, bis-ethylhexyloxyphenol methoxyphenyl triazine, drometrizole trisiloxane, bisdisulizole disodium, disodium phenyl dibenzimidazole tetrasulfonate, diethylamino hydroxybenzoyl hexyl benzoate, octyl triazone, ethylhexyl triazone, iscotrizinol, diethylhexyl butamido triazone, polysilicone-15, hydroxyphenylbenzotriazole, isoamyl p-methoxycinnamate, benzophenone-3 (oxybenzone), benzophenone-4 (sulisobenzone), benzophenone-8 (dioxybenzone), butyl methoxydibenzoylmethane (avobenzone), cinoxate, DEA-methoxycinnamate, digalloyl trioleate, dimethicone/PEG-15 crosspolymer, 1-(3,4-dimethoxyphenyl)-4,4-dimethyl-1,3-pentanediene, ethylhexyl methoxycinnamate (octinoxate), ethylhexyl salicylate (octisalate), 4-(2-beta-glucopyranosiloxy) propoxy-2-hydroxybenzophenone, Helianthus annuus (sunflower) seed extract, homosalate, hydrolyzed linseed extract, lawsone, menthyl anthranilate (meradimate), octocrylene, phenylbenzimidazole sulfonic acid (ensulizole), Pinus pinaster bark extract, red petrolatum, Spirulina platensis powder, TEA-salicylate (trolamine salicylate), titanium dioxide, Vitis vinifera (grape) seed extract, and combinations thereof. The inventive composition may include 0.0001% to approximately 40% by weight of the sunscreen agent. In certain embodiments, the composition includes approximately 0.0001% to approximately 5% by weight of the sunscreen agent. In certain embodiments, the composition includes approximately 0.001% to approximately 5% by weight of the sunscreen agent. In certain embodiments, the composition includes approximately 0.0005% to approximately 3% by weight of the sunscreen agent. In certain embodiments, the composition includes approximately 0.001% to approximately 2% by weight of the sunscreen agent. In certain embodiments, the composition does not include a sunscreen agent.

### Vitamins

The inventive cosmetic compositions may include a vitamin to nourish or replenish the treated skin. Exemplary vitamins include vitamin A, vitamin B₁ (thiamine), vitamin B₂ (riboflavin), vitamin B₃ (niacin), vitamin B₄ (adenine), vitamin B₅ (pantothenic acid), vitamin B₆ (pyridoxine), vitamin B₇ (biotin), vitamin B₉ (folic acid), vitamin B₁₂ (cyanocobalamin), vitamin C (ascorbic acid), vitamin D (ergocalciferol), vitamin E (tocopherol), vitamin K, and combinations thereof. Salts, esters, and other forms of a vitamin are also acceptable for use in the invention. In certain embodiments, the cosmetic composition includes vitamin E. In certain embodiments, the cosmetic composition includes tocopheryl acetate. In certain embodiments, the cosmetic composition includes one of the B vitamins (e.g., vitamin B₅). In certain embodiments, the compositions includes vitamin C or a derivative thereof (e.g., ascorbyl palmitate). The inventive composition may include 0.0001% to approximately 20% by weight of the vitamin. In certain embodiments, the composition includes approximately 0.0001% to approximately 5% by weight of the vitamin. In certain embodiments, the composition includes approximately 0.001% to approximately 5% by weight of the vitamin. In certain embodiments, the composition includes approximately 0.0005% to approximately 3% by weight of the vitamin. In certain embodiments, the composition includes approximately 0.001% to approximately 2% by weight of the vitamin.

### Dyes/Coloring Agents

The inventive cosmetic compositions may include a dye or other coloring agent. Any dye or coloring agent approved for use on humans by the U.S. Food and Drug Administration may be used in the inventive cosmetic compositions. In certain embodiments, the dye or coloring agent is a FD&C or D&C dye, lake, or pigment. Such materials are well known in the art. Exemplary dyes and coloring agents include 1,7-dihydroxyaphthalene; 1,3-diaminobenzene; 1-methyl-2,5-diaminobenzene; 1,4-diaminobenzene; 1,3-Dihydroxybenzene; 1,3-Benzenediol, 4-chloro-; 1-Hydroxy-2-aminobenzene; 3-amino-phenol; 1-Hydroxy-4-aminobenzene; 1-Hydroxynaphthalene; 1,5-Dihydroxynaphthalene; 2,7-Dihydroxynaphthalene; 1,4-Dihydroxybenzene; 1-Hydroxy-4-methylaminobenzene; 6-Hydroxybenzomorpholine; 1-Methyl-2-hydroxy-4-aminobenzene; 1-Methyl-2-hydroxy-4-(2′-hydroxyethyl)aminobenzene; 1-Phenyl-3-methylpyrazol-5-on; 1-(2′Hydroxyethyloxy)-2,4-diaminobenzene hydrochloride; 1,3-Dihydroxy-2-methylbenzene; 1-Amino-4-bis-(2′-hydroxyethyl)aminobenzene sulfate; 1-Hydroxy-3-methyl-4-aminobenzene; 1-Hydroxy-2-amino-5-methylbenzene; 1-(2′-Hydroxyethyl)2,5-diaminobenzene sulfate; 1-Methoxy-2-amino-4-(2′-hydroxyethylamino)benzene; 3,5-Pyridinediamine, 2,6-dimethoxy-, dihydrochloride; 4-Amino-2-aminomethylphenol HCl; 6-Hydroxyindole; 2,3-Indolinedione; 2-Amino-3-hydroxypyridine; 2,4-Dinitro-3′-sulfo-4′-phenylaminodiphenylamine; 1-(4′-Nitrophenylazo)-2-methyl-4-bis-(betahydroxyethyl)aminobenzene; Mixture of 1-(3-Nitro-4-amino)-phenylazo-2-hydroxy-7-trimethylammoniumchloride naphthalene and 1-(2′-nitro-4′-amino)-phenylazo-2-hydroxy-7-trimethylammoniumchloride naphthalene; 1-Amino-2-(4′-nitrophenylazo)-7-phenylazo-8-hydroxynapthalene-3,6-disulfonic acid, Disodium salt; 1-Amino-4-methylaminoanthraquinone; 1,2-Diamino-4-nitrobenzene; 1,4-Diamino-2-nitrobenzene; 1-Hydroxy-2-amino-4,6-dinitrobenzene; 1,4-Bis-(2′-Hydroxyethyl)-amino-2-nitrobenzene; 1-Amino-2-(b-hydroxyethyl)-amino-5-nitrobenzene; Ethanol, 2-[(4-amino-3-nitrophenyl)amino]-; Ethanol, 2,2′-[[4-[(2-hydroxethyl)amino]-3-nitrophenyl]imino]bis-; N,O-Di(hydroxyethyl)-2-amino-5-nitrophenol; 1-(2′-Hydroxyethyl)amino-2-nitrobenzene; 2-Nitro-4′-hydroxydiphenylamine; 2-Nitro-4-aminodiphenylamine; 2-Chloro-5-nitro-n-hydroxyethyl-p-phenylenediamine; 1-(2′Hydroxyethyl)amino-2-nitro-4-aminobenzene; 1-Hydroxy-3-nitro-4-aminobenzene; 1-methoxy-2-(2′hydroxyethyl)-amino-5-nitrobenzene; 1-Hydroxy-3-nitro-4-(2′-hydroxyethyl)aminobenzene; 1-Hydroxy-2-amino-3-nitrobenzene; 1-Amino-2-methyl-6-nitrobenzene; 1-(2′-Hydroxyethyl)oxy-3-methylamino-4-nitrobenzene; 1-Methylamino-2-nitro-5-(2′,3′-dihydroxypropyl)oxybenzene; 1-(2′-Hydroxyethyl)amino-2-hydroxy-4-nitrobenzene; 1-Amino-3-methyl-4(2-hydroxyethyl)amino-6-nitrobenzene; 1,2-Ethanediamine, N-(5-methoxy-2-nitrophenyl)-, monohydrochloride; 1-(2′-Ureldoethyl)amino-4-nitrobenzene; Mixture of 1,2-Propanediol, 3-[(4-amino-2-chloro-5-nitrophenyl)amino]- and 1,2-Propanediol, 3,3′-[(2-chloro-5-nitro-1,4-phenylene)diimino]bis-; Ethanol, 2-[4-[ethyl[(2-hydroxyethyl)amino]-2-nitrophenyl]amino]-hydrochloride; 1-Hydroxy-2-(2′-hydroxyethylamino)-4,6-dinitrobenzene; 4-(2′,3′-Dihydroxypropyl)amino-3-nitrotrifluormethylbenzene; 1-Methyl-3-nitro-4-(2′-hydroxyethyl)aminobenzene; 1-Chloro-3-nitro-4-(2′-hydroxyethyl)aminobenzene; 1-(4′-Aminophenylazo)-2-methyl-4-bis(2′-hydroxethyl)aminobenzene; 2-Chloro-6-ethylamino-1-hydroxy-4-nitrobenzene; 2,5-Diamino-6-nitropyridine; 2-Amino-6-chloro-4-nitrophenol; 1-Hydroxy-3-nitro-4-(3-hydroxypropylamino)benzene; Arianor bordeaux; 1H-Imidazolium, 2-[[4-(dimethylamino)phenyl]azo]-1,3-dimethyl-,chloride; Pyridinium, 1-methyl-4-[(methylphenylhydrazono)methyl]-, methyl sulfate; 1H-Imidazolium, 2-[(4-aminophenyl)azo]-1,3-dimethyl-,chloride; Pyridinium, 1-methyl-4-[(methylphenylhydrazono)methyl]-, methyl sulfate; 1H-Imidazolium, 2-[(4-aminophenyl)azo]-1,3-dimethyl,chloride; 5-(4′-dimethylaminophenylazo)-1,4-dimethyl-triazolium chloride; 1-(2′-Methoxyphenylazo)-2-hydroxy-7-trimethyl-ammoniumnaphthalene-chloride; 1-(4′-Amino-phenylazo)-2-hydroxy-7-trimethylammoniumnaphthalene; 3-[(4,5-dihydro-3-methyl-5-oxo-1-phenyl-1H-pyrazol-4-yl)azo]-N,N,N-trimethylanilinium chloride; 7-(2′,4′-Dimethyl-5′-sulfophenylazo)-5-sulfo-8-hydroxynaphthalene; 1-(4′-Sulfophenylazo)-2-hydroxy-naphthalene; 7-Phenylazo-1-amino-3,6-disulfo-8-hydroxy-naphthalene; 7-(6′-Methylphenylazo)-1-acetamido-3,6-disulfo-8-hydroxy-naphthalene; 4-(4′-Sulfo-1-phenylazo)-1-(4-sulfophenyl)-3-carboxy-5-hydroxy-pyrazolone; 4,4′-Bis-(N(Ethyl, 3 sulfobenzyl)-amino-2-sulfofuchsonium; 4′,4′,4-Triamino-2-methyl-triphenylcarbeniumchloride; Bis-4,4′-Diethylaminophenyl-4-ethylaminonaphthyl-carbenium chloride; Bis-4,4′-Dimethylaminophenyl-4-phenylaminonaphthyl carbenium chloride; Sodium salt of mixture of mono- & disulfonic acids (mainly the latter) of quinophthlanone or 2-quinolylindandione; 2,8-Dimethyl-3,7-diamino-5-phenylphenazinium-chloride; 2-Bromo-4,8-diamino-6-(3-trimethylammonium)-phenylamino-1,5-naphthoquinone; 1-Amino-4-hydroxy-anthraquinone; 1-)2′-Sulfo-4′-methylphenyl)-amino-4-hydroxy-anthraquinone; 1,4-Diaminoanthraquinone; 1-Amino-2-sulfo-4-cyclohexylamino-anthraquinone; 4-Bis-(2-Hydroxyethlyamino)-4′-amino-benzene Ethanol, 2,2′-[[4-[(4-aminophenyl)azo]phenyl]imino]bis; 1-(N-Methylmorphollnium-propylamino)-4-hydroxy-anthraquinone methylsulfate; 1-Methylamino-4-amino-propylamino-anthraquinone; 1-Gamma-amino-propylamino-anthraquinone; 2-Hydroxy-1,4-naphthoquinone; 6-Hydroxy-5-[(2-methoxy-5-methyl-4-sulfo-phenyl)azo]-2-naphthalensulfonic acid, disodium salt; 7-Hydroxy-8-[(4-sulfo-1-naphthalenyl)azo]-1,3-naphthtlenedisulfonic acid, trisodium salt; 4,4′-Bis-(N(Ethyl,3 sulfobenzyl)-amino-2-sulfo-4-hydroxy-fuchsonlum, Disodium salt; Hydrogen 3,6-bis(diethylamino)-9-(2,4-disulphonatophenyl)xanthylium, sodium salt; 1,4 Di-[-(2′-sulfo-4′-methylphenyl)-amino]-anthraquinone; 1-Hydroxyethyl-4,5-diamino pyrazole sulfate; (3-ammonio-4-methoxyphenyl)(2-hydroxyethyl)ammonium sulphate; 2-(1,3-benzodioxol-5-ylamino)ethanol hydrochloride; 3,4-(methylenedioxy)phenol; 4,4′-[propane-1,3-diylbis(oxy)]bisbenzene-1,3-diamine tetrahydrochloride; 2-Chloro-6-methyl-3-aminophenol HCI; 2,2′-[(4-amino-3-nitrophenyl)imino]bisethanol hydrochloride; 2,4,5,6-Tetraminopyrimidine Sulfate; 2,6-dihydroxy-3,4-dimethylpyridine; Ethanol, 2,2′-[(2-nitro-1,4-phenylene)diimino]bis-; 1,4-benzenediamine, 2-nitron-n(1)-(2-carboxyphenyl)-; Phenol, 2-aminomethyl-4-amino-, dihydrochloride; Benzene-1,4-diamine dihydrochloride; 4-amino-3-nitrophenol; Benzene-1,3-diammonium sulphate; 2,5,6-triaminopyrimidin-4-ol sulphate; 2-amino-6-chloro-4-nitrophenol monohydrochloride; 4-[(4-amino-m-tolyl)(4-imino-3-methylcyclohexa-2,5-dien-1-ylidene)methyl]-o-toluidine monohydrochloride; 9,10-Anthracenedione, 1,4-diamino-, N,N′-mixed 2-hydroxyethyl and Methyl derives; Sodium 2-amino-4,6-dinitrophenoxide; 2-methyl-p-phenylenediamine sulphate; 4,4′-[1,3-propanediylbis(oxy)]bisbenzene-1,3-diamine; 1-(3-Hydroxypropylamino)-4-bis-(2-hydroxyethylamino)benzene HCI; 3-amino-2-chloro-6-methylphenol; 1,3-diamino-4-(2-hydroxyethoxy)benzene sulphate; 9,10-Anthracenedione, 1,4-dihydroxy-5,8-bis[(2-hydroxyethyl)amino]-; 1,5-di-(2-hydroxyethyl)-amino-2-nitro-4-chlorobenzene; 1-(3-Hydroxypropylamino)-2-nitro-4-bis-(2-hydroxyethylamino)benzene; 1,4-Bis-(2,3-dihydroxy propyl)-amino-anthraquinone; 2,6-Diamino-3-((pyridine-3-yl)azo)pyridine; 2-[2-Nitro-4-(trifluoromethyl)phenylamino]ethanol; Disodium 5,7-dinitro-8-oxidonaphthalene-2-sulphonate; 3,4,5,6-Tetrachloro-2-(1,4,5,8-Tetrabromo-6-hydroxy-3-oxoxanthen-9-yl)benzoic acid; Disodium-3-hydroxy-4-[4-methyl-2-sulphonatophenylazo]-2-Naphthoate; 4,4′-(4,5,6,7-tetrabromo-3H-2,1-benzoxathiol-3-ylidene)bis[2,6-dibromophenol]S,S-dioxide; 4-[(2,6-dichlorophenyl)(4-imino-3,5-dimethylcyclohexa-2,5-dien-1-ylidene)methyl]-2,6-xylidine phosphate (1:1); Phenol, 3-amino-2,4-dichloro-hydrochloride; Phenol, 2-amino-4-chloro-; Ethanol, 2,2′-[(2-methyl-1,3-phenylene)diimino]bis-; 6-methoxy-2-methylamino-3-aminopyridine HCI; 1-Hydroxy-4-methylaminobenzene sulfate; bis-(5-Amino-2-hydroxyphenyl)-methane (2HCl); 1-[(2-Chloro-4-nitrophenyl)azo]-2-naphthalenol; and 1,2,3,4-tetrahydro-6-nitroquinoxaline (HCl). Any colorant listed in 21 C.F.R. § 178.3297 may be used in an inventive cosmetic composition. The inventive composition may include 0.0001% to approximately 5% by weight of the dye or coloring agent. In certain embodiments, the composition includes approximately 0.001% to approximately 5% by weight of the dye or coloring agent. In certain embodiments, the composition includes approximately 0.01% to approximately 3% by weight of the dye or coloring agent. In certain embodiments, the composition includes approximately 0.1% to approximately 5% by weight of the dye or coloring agent. In certain embodiments, the inventive cosmetic composition does not include a dye or other coloring agent.

### Proteins/Amino Acids

The inventive cosmetic compositions may include a protein, peptide, or amino acid. Such components may be added to the inventive composition to nourish the skin, impart a desired characteristic to skin, or impart a desired characteristic to the composition (e.g., thickening the composition). Exemplary proteins that may be added to inventive compositions include elastin, fibrillin, fibronectin, laminin, keratin, proteoglycans, wheat protein, gelatin, collagen, silk, soy protein, wheat protein, rice protein, corn protein, jojoba protein, milk protein, whey protein, casein, albumin, egg protein, and fragments thereof. As would be appreciated by one of ordinary skill in the art, derivatives, mutants, fusion proteins, fragments, or combinations of any of these proteins may also be included in the inventive cosmetic composition. In certain embodiments, any of the twenty natural amino acids may be included in the inventive cosmetic composition. In certain embodiments, the amino acid used in the inventive composition is selected from the group consisting of phenylalanine, valine, tryptophan, threonine, isoleucine, methionine, cysteine, homocysteine, histidine, arginine, lysine, leucine, and combinations thereof. In certain embodiments, the amino acid(s) used in the composition is an essential amino acid (e.g., isoleucine, leucine, lysine, methionine, phenylalanine, threonine, tryptophan, valine, arginine, and/or histidine). In certain embodiments, the amino acid(s) used in the composition is a nonessential amino acid (e.g., alanine, asparagine, aspartate, cysteine, glutamine, glutamate, glycine, proline, serine, and/or tyrosine). In certain embodiments, cysteine, homocysteine, or methionine is included in the cosmetic composition. In certain embodiments, short peptides (e.g., dipeptides, tripeptides, quatrapeptide, pentapeptides, etc.) are included in the cosmetic composition. Proteins, peptides, or amino acids may be added to the inventive hair care compositions up to 10% by weight. In certain embodiments, the proteins, peptides, or amino acids are included in the composition in a range from about 0.0001% to about 10%. In certain embodiments, the proteins, peptides, or amino acids are included in the composition in a range from about 0.01% to about 5%. In certain embodiments, the proteins, peptides, or amino acids are included in the composition in a range from about 0.1% to about 3%. In certain embodiments, the proteins, peptides, or amino acids are included in the composition in a range from about 0.1% to about 2%. In certain embodiments, the proteins, peptides, or amino acids are included in the composition in a range from about 5% to about 10%. In certain embodiments, the proteins, peptides, or amino acids are included in the composition in a range from about 1% to about 5%.

### Plant Extracts

The inventive cosmetic compositions may include an extract from a plant. Plant extract may be added to the inventive composition to nourish the skin, provide a fragrance or color to the composition, impart a desired characteristic to treated skin, or impart a desired characteristic to the composition. Extracts may be prepared from any part of a plant, including leaves, fruit, flower, grass, vegetable, nut, root, stem, bark, and the like. An extract may be prepared by using any solvent and optionally heat. The extraction solvents are typically polar organic solvents including, for example, lower alcohols such as methanol, ethanol, and the like, propylene glycol, 1,3-butylene glycol and glycerin, and water. These solvents may be used singly or in combination. Any extraction technique known in the art may be used to prepare the plant extract. Any plant may be used to prepare a plant extract. Exemplary plants that can be used to prepare plant extracts include, but are not limited to, birch, rosemary, arnica, hamamelis, camomile, sage, St. John's bread, henna, hop, lime, orange, lemon, grapefruit, aloe, thyme, calendula, horsetail, mountain gentian, nettle, chestnut, avocado, seaward, milfoil, coltsfoot, marigold, peach, rose, senna, mint, white lilly, lavender, grape seed, bayberry, saw palmetto, tea tree, soy bean, almond, peanut, sea buckthorn seed, seaweed, tea tree, hibiscus, lemongrass, horsetail, raspberry, rose hips, and olive. Certain of these plants have been used in the past to prepare plant extract for cosmetic compositions. In certain embodiments, the plant extracts useful in the inventive composition may be obtained commercially. When the plant extract is in a liquid form, the plant extract is typically used in an amount ranging from 0.001 to 10.0% by weight of the total composition, calculated as a residue obtained after distillation of extraction solvent therefrom. In certain embodiments, the plant extract is used in an amount ranging from 0.01 to 1.0% by weight of the total composition, calculated as a residue obtained after distillation of extraction solvent therefrom.

### Humectants

The inventive cosmetic compositions may include a humectant. A humectant is a hydrogroscopic substance. It is typically a chemical compound containing hydrophilic groups such as hydroxyl groups, amines, carboxylates, amides, esters, etc. Humectants are typically found in cosmetic compositions to reduce static and/or to provide a moisturizing quality to the composition. The humectants attracts and holds moisture on the skin. Examples of humectants useful in the inventive compositions include acetyl glucosamine, ascorbic acid, diglycerin, erythritol, fructose, glucose, inositol, lactitol, lactose mannose, methylglucamine, methylpropanediol, phytantriol, raffinose, riboflavin, maltose, glycerin, glycerol, hyaluronic acid, atelocollagen, propylene glycol, glyceryl triacetate, polyols, sorbitol, sorbityl acetate, sorbityl furfural, sorbityl silanediol, xylose, tromethamine, zinc glucoheptonate, xylitol, maltitol, polydextrose, quillaia, lactic acid, sodium lactate, triacetin, urea, Acetyl Arginine, Acetyl Hydroxyproline, Adansonia Digitata Fruit Extract, Adenophora Stricta Root Extract, Agave Atrovirens Extract, Alanyl Glutamine, Alcaligenes Polysaccharides, Algae Extract, Aloe Barbadensis Leaf Extract, Aloe Barbadensis Leaf Polysaccharides, Amidinoproline, Bacillus/Rice Bran Extract/Soybean Extract Ferment Filtrate, Betaine, Bittern, Black Strap Powder, 2,3-Butanediol, Caprylyl Glycol/Glycerin/Polyacrylic Acid Copolymer, Carnitine HCI, Chitosan Lauroyl Glycinate, Cholesterol/HDI/Pullulan Copolymer, Coix Lacryma-Jobi (Job's Tears) Shell Extract, Coleus Forskohlii Root Extract, Cryptomeria Japonica Leaf Extract, Cucumis Melo (Melon) Fruit Water, Diglycereth-7 Malate, Diglycerin, Diglycol Guanidine Succinate, Dunaliella Bardawil Extract, Earthworm Conditioned Soil, Earthworm Conditioned Soil Extract, Erythritol, Ethylhexyl Hydroxystearoyl Hydroxystearate, Fructose, Glucose, Glucosyl Hesperidin, Glucuronolactone, Glycereth-7 Glycolate, Glycerin, Glyceryl Citrate Crosspolymer, Glyceryl Dimaltodextrin, Glycol, Glycosyl Trehalose, Hesperetin Laurate, 1,2,6-Hexanetriol, Honey, Hydrogenated Honey, Hydrogenated Isocetyl Olivate, Hydrogenated Starch Hydrolysate, Hydrolyzed Wheat Protein/PEG-20 Acetate Copolymer, Hydroxypropyltrimonium Hyaluronate, Impatiens Balsamina Extract, Inositol, Isostearyl Acetyl Glutaminate, Jojoba Amino Acids, Lactic Acid, Lactitol, Lactobaccillus/Phoenix Dactylifera (Date) Fruit Ferment Extract, Lauramidobutyl Guanidine Acetate, Lauramidobutyl Guanidine HCI, Laur/MyrisUPalmitamidobutyl Guanidine Acetate, Lauryl Malamide, Lonicera Caerulea Fruit Juice, Lonicera Caerulea Fruit Water, Lysophosphatidylethanolamine, Malpighia Emarginata (Acerola) Seed Extract, Maltitol, Maltose, Mannitol, Mannose, Methoxy PEG-7, Methoxy PEG-10, Methoxy PEG-16, Methoxy PEG-25, Methoxy PEG-40, Methoxy PEG-100, Momordica Charantia Fruit Extract, Myrciaria Dubia Seed Extract, Myristamidobutyl Guanidine Acetate, Myrist/Palmitamidobutyl Guanidine Acetate, Palmitamidobutyl Guanidine Acetate, PEG-4, PEG-6, PEG-7, PEG-8, PEG-9, PEG-10, PEG-12, PEG-14, PEG-16, PEG-18, PEG-20, PEG-32, PEG-33, PEG-40, PEG-45, PEG-55, PEG-60, PEG-75, PEG-80, PEG-90, PEG-100, PEG-135, PEG-150, PEG-180, PEG-200, PEG-220, PEG-240, PEG-800, PEG-15 Butanediol, PEG-3 Methyl Ether, PEG-4 Methyl Ether, PEG-8 Methyl Ether Dimethicone, PEG-5 Pentaerythrityl Ether, PEG/PPG-28/21 Acetate Dimethicone, PEG/PPG-23/23 Butyl Ether Dimethicone, PEG/PPG-24/18 Butyl Ether Dimethicone, Persea Thunbergii Extract, Polydimethylsiloxy PPG-13 Butyl Ether Silsesquioxane, Polyglyceryl Sorbitol, Polyquaternium-64, Polyquaternium-65, Potassium Dextrin Octenylsuccinate, Potassium PCA, PPG-12 Butyl Ether Dimethicone, PPG-6-Sorbeth-245, PPG-6-Sorbeth-500, PPG-2 Tocophereth-5, Propylene Glycol, Prunus Mume Fruit Extract, Pseudoalteromonas Ferment Extract, Rosa Canina Seed Extract, Saccharomyces/Prunus Extract Ferment Filtrate, Schizophyllan, Sea Water, Sodium Acetylated Hyaluronate, Sodium Dextrin Octenylsuccinate, Sodium Glucuronate, Sodium PCA, Sorbeth-6, Sorbeth-20, Sorbeth-30, Sorbeth-40, Sorbitol, Sorbityl Silanediol, Stearyl Acetyl Glutaminate, Stearyl Palmitate, Sucrose, TEA-Dextrin Octenylsuccinate, Trehalose, Triglycereth-7 Citrate, Trimethylamine Oxide, Trioxaundecanedioic Acid, Tripropylene Glycol, Urea, Urea-d-Glucuronic Acid, Xylitol, Xyloglucan, and Xylose. The humectant is used in the composition in an amount ranging from about 0.01% to about 30% by weight. In certain embodiments, the humectant is used in the cosmetic composition in an amount ranging from about 0.1% to about 20% by weight. In certain embodiments, the humectant is used in the cosmetic composition in an amount ranging from about 1% to about 25% by weight. In certain embodiments, the humectant is used in the cosmetic composition in an amount ranging from about 1% to about 10% by weight. In certain embodiments, the humectant is used in the cosmetic composition in an amount ranging from about 1% to about 5% by weight.

### Fragrances/Perfumes

The inventive cosmetic compositions may optionally include a fragrance or perfume. Fragrance ingredient, as defined by the International Fragrance Association, is “any basic substance used in the manufacture of fragrance materials for its odorous, odor-enhancing, or blending properties. Fragrance ingredients may be obtained by chemical synthesis from synthetic, fossil, or natural raw materials, or by physical operations from natural sources. The function comprises aroma chemicals, essential oils, natural extracts, distillates and isolates, oleoresins, etc.” In certain embodiments, a fragrance is any natural or synthetic substance or substances used solely to impart an odor to a cosmetic product. Any perfume or fragrance used in the cosmetics industry may be used in the inventive hair care compositions. In certain embodiments, the perfume or fragrance is commercially available. In certain embodiments, the perfume or fragrance may be blended from raw ingredients used in the cosmetics industry. Exemplary fragrance ingredients include Abies Alba Leaf Oil, Abies Balsamea (Balsam Canada) Resin, Abies Pectinata Oil, Abies Sibirica Oil, Acacia Catechu Gum, Acacia Senegal Gum, Acetaldehyde, Acetanilid, Acetic Acid, 1-Acetonaphthone, 2-Acetonaphthone, Acetone, Acetyl Hexamethyl Indan, Acetyl Hexamethyl Tetralin, Acetyl Tributyl Citrate, Acetyl Triethyl Citrate, Achillea Millefolium Extract, Achillea Millefolium Flower Water, Achillea Millefolium Oil, Achyrocline Satureiodes Flower Oil, Actinidia Chinensis (Kiwi) Fruit Water, Adipic Acid, Agar, Agropyron Repens Root Extract, Alanine, Albizia Julibrissin Bark Extract, Alcohol, Alcohol Denat., Algae Extract, Algin, Allyl Caproate, Aloe Barbadensis Leaf, Aloe Barbadensis Leaf Water, Amidinoproline, Aminomethyl Propanediol, Ammonia, Ammonium Chloride, Ammonium Glycyrrhizate, Amyl Acetate, Amyl Benzoate, Amyl Cinnamal, Amylcinnamyl Alcohol, Amyl Salicylate, Amyris Balsamifera Bark Oil, Anethole, Angelica Archangelica Root Extract, Angelica Archangelica Root Oil, Aniba Rosaeodora (Rosewood) Wood Extract, Aniba Rosaeodora (Rosewood) Wood Oil, Anisaldehyde, Anise Alcohol, p-Anisic Acid, Anthemis Nobilis Flower Extract, Anthemis Nobilis Flower Oil, Apium Graveolens (Celery) Extract, Arginine, Arnica Montana Flower Extract, Artemisia Annua Extract, Artemisia Princeps Leaf Water, Artemisia Tridentata Oil, Ascorbic Acid, Ascorbyl Palmitate, Asparagine, Aspartic Acid, Astragalus Gummifer Gum, Azelaic Acid, Backhousia Citriodora Oil, Beeswax, Benzaldehyde, Benzoic Acid, Benzophenone, Benzophenone-2, Benzophenone-6, Benzyl Acetate, Benzyl Alcohol, Benzyl Benzoate, Benzyl Benzoyloxybenzoate, Benzyl Cinnamate, 3-Benzylidene Camphor, Benzyl Laurate, Benzyl Salicylate, Betula Alba Bark Extract, Betula Alba Extract, Betula Alba Leaf Extract, Betula Alba Oil, BHA, BHT, Bisabolol, Bixa Orellana Extract, Bixa Orellana Seed Extract, Boesenbergia Pandurata Root Oil, Borneo), Boswellia Carterii Extract, Boswellia Carterii Oil, Boswellia Serrata Gum, Boswellia Serrata Oil, 2,3-Butanediol, Butoxydiglycol, Butoxyethanol, Butoxyethyl Acetate, Butyl Acetate, n-Butyl Alcohol, t-Butyl Alcohol, 4-t-Butyl Benzaldehyde, Butyl Benzoate, 4-t-Butylbenzoic Acid, 2-t-Butylcyclohexyl Acetate, 2-t-Butylcyclohexyloxybutanol, Butylene Glycol, Butyl Lactate, Butyl Methacrylate, Butyl Oleate, Butylparaben, Butylphenyl Methylpropional, Butyl Stearate, Butyric Acid, Butyrolactone Caffeic Acid, Caffeine, Calcium Acetate, Calcium Alginate, Calendula Officinalis Flower Extract, Calendula Officinalis Flower Oil, Callitris Introtropica Wood Oil, Callitris Quadrivalvis Gum, Camellia Oleifera Leaf, Camellia Sinensis Leaf Extract, Camellia Sinensis Leaf Water, Camphene, Camphor, Camphylcyclohexanol, Cananga Odorata Flower Oil, Cananga Odorata Flower Wax, Canarium Commune Gum Oil, Canarium Luzonicum Gum Nonvolatiles, Capric Acid, Caproic Acid, Gamma-Caprolactone, Caprylic Acid, Caprylic Alcohol, Caprylic/Capric Triglyceride, Caprylyl Butyrate, Capsaicin, Capsicum Annuum Extract, Capsicum Frutescens Resin, Caramel, Carmine, Carthamus Tinctorius (Safflower) Seed Oil, Carum Carvi (Caraway) Fruit Oil, Carum Carvi (Caraway) Seed Extract, Carum Carvi (Caraway) Seed Oil, Carom Petroselinum (Parsley) Seed Oil, Carvone, Beta-Caryophyllene, Cedrol, Cedrus Atlantica (Cedarwood) Bark Oil, Cedrus Deodara Wood Oil, Cellulose Gum, Ceratonia Siliqua (Carob) Fruit Extract, Ceratonia Siliqua Gum, Cetyl Acetate, Cetyl Alcohol, Cetyl Palmitate, Chamaecyparis Obtusa Oil, Chamaecyparis Obtusa Water, Chamomilla Recutita (Matricaria) Flower Extract, Chamomilla Recutita (Matricaria) Flower Oil, Chamomilla Recutita (Matricaria) Flower Water, Chamomilla Recutita (Matricaria) Leaf Extract, Chondrus Crispus (Carrageenan), Chouji Yu, CI 75470, Cichorium Intybus (Chicory) Leaf Extract, Cichorium Intybus (Chicory) Root Extract, Cinnamal, Cinnamomum Camphora (Camphor) Bark Oil, Cinnamomum Camphora (Camphor) Leaf Extract, Cinnamomum Cassia Leaf Oil, Cinnamomum Zeylanicum Bark Oil, Cinnamyl Acetate, Cinnamyl Alcohol, Cistus Ladaniferus Extract, Cistus Ladaniferus Oil, Cistus Ladaniferus Resin, Cistus Monspeliensis Extract, Citral, Citric Acid, Citronella), Citronellol, Citronellyl Acetate, Citronellyl Methylcrotonate, Citrus Aurantifolia (Lime) Oil, Citrus Aurantium Amara (Bitter Orange) Flower Water, Citrus Aurantium Amara (Bitter Orange) Oil, Citrus Aurantium Amara (Bitter Orange) Peel Extract, Citrus Aurantium Bergamia (Bergamot) Fruit Oil, Citrus Aurantium Dulcis (Orange) Flower Oil, Citrus Aurantium Dulcis (Orange) Flower Water, Citrus Aurantium Dulcis (Orange) Fruit Extract, Citrus Aurantium Dulcis (Orange) Fruit Water, Citrus Aurantium Dulcis (Orange) Oil, Citrus Grandis (Grapefruit) Fruit Water, Citrus Grandis (Grapefruit) Peel Oil, Citrus Grandis/Paradisi Fruit Water, Citrus Limon Leaf Oil, Citrus Medica Limonum (Lemon) Fruit Extract, Citrus Medica Limonum (Lemon) Fruit Water, Citrus Medica Limonum (Lemon) Peel Oil, Citrus Medica Vulgaris Peel Oil, Citrus Nobilis (Mandarin Orange) Fruit Extract, Citrus Nobilis (Mandarin Orange) Peel Extract, Citrus Nobilis (Mandarin Orange) Peel Oil, Citrus Reticulata Leaf Oil, Citrus Tangerina (Tangerine) Peel Oil, Citrus Unshiu Peel Powder, Cnidium Officinale Root Water, Cochlearia Armoracia (Horseradish) Root Extract, Cocos Nucifera (Coconut) Oil, Cocos Nucifera (Coconut) Water, Cod Liver Oil, Coffea Arabica (Coffee) Extract, Coffea Arabica (Coffee) Seed Extract, Coffea Arabica (Coffee) Seed Oil, Coleus Forskohlii Root Oil, Commiphora Myrrha Oil, Commiphora Myrrha Resin, Copaifera Officinalis (Balsam Copaiba) Resin, Coriandrum Sativum (Coriander) Extract, Coriandrum Sativum (Coriander) Fruit Oil, Corylus Avellana (Hazel) Leaf Water, Corylus Avellana (Hazel) Seed Oil, Coumarin, Crataegus Oxyacantha Flower Water, m-Cresol, o-Cresol, p-Cresol, Crocus Sativus Flower Extract, Crotonaldehyde, Crotonic Acid, Cryptocarya Crassinervia Bark Oil, Cryptocarya Massoy Bark Extract, Cuminum Cyminum (Cumin) Seed Extract, Cuminum Cyminum (Cumin) Seed Oil, Cupressus Sempervirens Oil, Curcuma Longa (Turmeric) Root Extract, Cyamopsis Tetragonoloba (Guar) Gum, Cyclamen Aldehyde, Cyclohexylethyl Acetate, Cyclohexylethyl Butyrate, Cyclopentadecanone, Cymbopogon Flexuosus Oil, Cymbopogon Martini Oil, Cymbopogon Nardus (Citronella) Oil, Cymbopogon Schoenanthus Oil, p-Cymene, Cyperus Esculentus Root Oil, Cysteine, Cystine, Cytisus Scoparius Flower Extract, Dalea Spinosa Seed Oil, Alpha-Damascone, Daucus Carota Sativa (Carrot) Root Water, Daucus Carota Sativa (Carrot) Seed Oil, Delta-Decalactone, Decanal, Decane, Decenal, Decenol, Decyl Alcohol, Denatonium Benzoate, Diacetone Alcohol, Dianthus Caryophyllus Flower Oil, Dibutyl Phthalate, Dibutyl Sebacate, Diethoxynonadiene, Diethyl Adipate, Diethylamine, Diethyl Caprylamide, Diethylene Glycol, Diethylhexyl Phthalate, Diethylhexyl Sebacate, Diethyl Oxalate, Diethyl Phthalate, Diethyl Sebacate, Diethyl Succinate, Dihydrocitronellol, Dihydrocoumarin, Dihydrojasmonate, Dihydro Pentamethylindanone, Diisobutyl Adipate, Diisopropyl Adipate, Dimethyl Brassylate, Dimethyl Carbonate, 2,4-Dimethyl-3-Cyclohexene Carboxaldehyde, Dimethyl Decadienal, Dimethyl Hexahydronaphthyl Dihydroxymethyl Acetal, Dimethylhydroxy Furanone, Dimethyloctahydro-2-Naphthaldehyde, 2,6-Diethyl-7-Octen-2-ol, Dimethyl Phenylpropanol, Dimethyl Phthalate, Dimethyl Succinate, Diphenyl Ether, Diphenyl Methane, Dipropylene Glycol, Dipteryx Odorata Seed Extract, Disodium Phosphate, Disodium Succinate, Dodecene, Eicosane, Elettaria Cardamomum Seed Extract, Elettaria Cardamomum Seed Oil, Erythorbic Acid, Ethoxydiglycol, Ethoxyethanol, Ethoxyethanol Acetate, Ethyl Acetate, Ethyl Benzoate, Ethyl Butyl Valerolactone, Ethylcellulose, Ethyl Cinnamate, Ethyl Cyclohexyl Propionate, Ethyl 2,2-Dimethylhydrocin namal, Ethylene Brassylate, Ethylene Dodecanedioate, Ethyl Ether, Ethyl Hexanediol, Ethylhexyl Ethylhexanoate, Ethylhexyl Palmitate, Ethylhexyl Salicylate, Ethyl Lactate, Ethyl Laurate, Ethyl Linoleate, Ethyl Linolenate, Ethyl Menthane Carboxamide, Ethyl Methacrylate, Ethyl Methylphenylglycidate, Ethyl Myristate, Ethyl Nicotinate, Ethyl Oleate, Ethyl Palmitate, Ethylparaben, Ethyl Pelargonate, Ethyl Phenethyl Acetal, Ethyl Phenylacetate, Ethyl Pyruvate, Ethyl Ricinoleate, Ethyl Stearate, Ethyl Thioglycolate, Ethyl Trimethylcyclopentene Butenol, Ethyl Vanillin, Eucalyptol, Eucalyptus Citriodora Oil, Eucalyptus Globulus Leaf Oil, Eucalyptus Globulus Leaf Water, Eugenia Caryophyllus Bud Oil, Eugenia Caryophyllus (Clove) Flower Extract, Eugenia Caryophyllus (Clove) Flower Oil, Eugenia Caryophyllus (Clove) Leaf Oil, Eugenia Operculata Leaf Powder, Eugenol, Eugenyl Acetate, Euphorbia Cerifera (Candelilla) Wax, Evernia Furfuracea (Treemoss) Extract, Evernia Prunastri (Oakmoss) Extract, Farnesene, Farnesol, Farnesyl Acetate, Ferula Galbaniflua (Galbanum) Resin Oil, Ficus Carica (Fig) Fruit Water, Foeniculum Vulgare (Fennel) Fruit Extract, Foeniculum Vulgare (Fennel) Fruit Powder, Foeniculum Vulgare (Fennel) Oil, Foeniculum Vulgare (Fennel) Water, Formic Acid, Fucus Vesiculosus Extract, Fumaric Acid, Furfural, Fusanus Spicatus Wood Oil, Galactoarabinan, Gardenia Florida Oil, Gaultheria Procumbens (Wintergreen) Leaf Extract, Gaultheria Procumbens (Wintergreen) Leaf Oil, Gentiana Lutea Root Extract, Geraniol, Geranium Maculatum Oil, Geranyl Acetate, Gluconic Acid, Gluconolactone, Glucose Pentaacetate, Glutamic Acid, Glutamine, Glutaral, Glutaric Acid, Glutathione, Glycerin, Glyceryl Oleate, Glyceryl Rosinate, Glyceryl Stearate, Glycine, Glycine Soja (Soybean) Oil, Glycol, Glycyrrhiza Glabra (Licorice), Glycyrrhizic Acid, Glyoxal, Gnaphalium Leontopodium Water, Gossypium Herbaceum (Cotton) Fruit Water, Guaiazulene, Hakka Yu, Hay Water, Helichrysum Stoechas Extract, Heliotropine, 2-Heptylcyclopentanone, Heterotheca Inuloides Flower Extract, Hexadecanolactone, Hexamethyl indanopyran, Hexanal, 3-Hexenol, Hexyl Alcohol, Hexyl Cinnamal, Hexylene Glycol, Hexyl Salicylate, Hibiscus Abelmoschuus Extract, Hinokitiol, Hippuric Acid, Histidine, Homosalate, Humulus Lupulus (Hops) Cone Oil, Humulus Lupulus (Hops) Extract, Hydroabietyl Alcohol, Hydrogenated Ethylbicycloheptane Guaiacol, Hydrogenated Lanolin, Hydrogenated Polydecene, Hydrogenated Rosin, Hydroquinone, p-Hydroxyanisole, 4-Hydroxybenzoic Acid, Hydroxycitronellal, Hydroxyisohexyl 3-Cyclohexene Carboxaldehyde, Hydroxymethoxybenzyl Pelargonamide, Hyptis Suaveolens Seed Oil, Hyssopus Officinalis Extract, Hyssopus Officinalis Leaf Oil, Illicium Verum (Anise) Oil, Iris Florentina Root Extract, Isoamyl Acetate, Isoamyl Alcohol, Isoamyl Allylglycolate, Isoamyl Cinnamate, Isoamyl Laurate, Isobornyl Acetate, Isobutenyl Methyltetrahydropyran, Isobutyl Acetate, Isobutyl Benzoate, Isobutyl Methyl Tetrahydropyranol, Isobutyl Palmitate, Isobutyl Pelargonate, Isobutyric Acid, Isododecane, Isoeugenol, Isoleucine, Isolongifolene Epoxide, Isolongifolene Ketone-Exo, Alpha-Isomethyl lonone, Isopentanal, Isopentylcyclohexanone, Isopropyl Acetate, Isopropyl Alcohol, Isopropyl Benzoate, Isopropyl Cyclohexylpropionate, Isopropyl Laurate, Isopropyl Myristate, Isopropyl Palmitate, Isopropylphenylbutanal, Isopulegol, Jasminum Officinale (Jasmine) Extract, Jasminum Officinale (Jasmine) Flower Extract, Jasminum Officinale (Jasmine) Flower Water, Jasminum Officinale (Jasmine) Oil, Juglans Regia (Walnut) Leaf Extract, Juglans Regia (Walnut) Shell Extract, Juniperus Communis Fruit Extract, Juniperus Communis Fruit Oil, Juniperus Mexicana Oil, Juniperus Oxycedrus Wood Oil, Juniperus Oxycedrus Wood Tar, Juniperus Scopulorum Oil, Juniperus Virginiana Oil, Keihi Ekisu, Keihi Yu, Ketoglutaric Acid, Kinginka Ekisu, Krameria Triandra Root Extract, Kunzea Ericoides Leaf Oil, Lactic Acid, Laminaria Cloustoni Extract, Laminaria Digitata Extract, Laminaria Hyperborea Extract, Laminaria Japonica Extract, Laminaria Ochroleuca Extract, Laminaria Saccharina Extract, Lantana Camara Leaf Water, Lauraldehyde, Lauramine Oxide, Lauric Acid, Laurus Nobilis Leaf, Laurus Nobilis Leaf Extract, Laurus Nobilis Oil, Lauryl Alcohol, Lauryl Lactate, Lavandula Angustifolia (Lavender) Extract, Lavandula Angustifolia (Lavender) Flower Extract, Lavandula Angustifolia (Lavender) Flower Powder, Lavandula Angustifolia (Lavender) Flower Water, Lavandula Angustifolia (Lavender) Oil, Lavandula Angustifolia (Lavender) Water, Lavandula Hybrida Extract, Lavandula Hybrida Oil, Lavandula Spica (Lavender) Extract, Lavandula Stoechas Extract, Lemon Ekisu, Leptospermum Petersonii Oil, Leptospermum Scoparium Oil, Leucine, Levisticum Officinale Oil, Levulinic Acid, Limonene, Linalool, Linalyl Acetate, Linoleic Acid, Linolenic Acid, Linum Usitatissimum (Linseed) Seed Oil, Lippia Citriodora Flower Water, Lippia Citriodora Water, Liquidambar Styraciflua Oil, Litsea Cubeba Fruit Oil, Longifolene, Lysine, Maleic Acid, Malic Acid, Malonic Acid, Marrubium Vulgare Extract, Massoy Bark Oil, Medicago Sativa (Alfalfa) Extract, MEK, Melaleuca Alternifolia (Tea Tree) Leaf Oil, Melaleuca Ericifolia Leaf Oil, Melaleuca Leucadendron Cajaput Oil, Melilotus Officinalis Extract, Melissa Officinalis Leaf Extract, Melissa Officinalis Leaf Oil, Mentha Aquatica Water, Mentha Arvensis Extract, Mentha Arvensis Leaf Oil, Mentha Arvensis Powder, p-Menthan-7-ol, Mentha Piperita (Peppermint) Leaf, Mentha Piperita (Peppermint) Leaf Extract, Mentha Piperita (Peppermint) Leaf Water, Mentha Piperita (Peppermint) Oil, Mentha Pulegium Oil, Mentha Viridis (Spearmint) Extract, Mentha Viridis (Spearmint) Leaf Oil, Menthol, Menthone Glycerin Acetal, Menthoxypropanediol, Menthyl Acetate, Menthyl Lactate, Menthyl Salicylate, Methionine, Methoxydiglycol, Methoxyethanol, Methoxyethanol Acetate, Methoxyindane, Methoxyisopropanol, Methoxytrimethylheptanol, Methyl Acetate, p-Methyl Acetophenone, Methylal, Methyl Alcohol, Methyl Anthranilate, 4-Methylbenzaldehyde, Methyl Benzoate, Methyl Benzodioxepinone, Methylbenzyl Acetate, Methyl 4-t-Butyl benzoate, Methyl Caproate, Methyl Caprylate, Methylcellulose, 6-Methyl Coumarin, Methylcyclohexenyl Butanol, Methylcyclopentadecenone, Methyldihydrojasmonate, Methyl Diisopropyl Propionamide, Methyl Eugenol, Methyl Hexyl Ether, Methyl Hydrogenated Rosinate, Methyl Lactate, Methyl Lactic Acid, Methyl Laurate, Methyl Linoleate, Methyl 3-Methyl resorcylate, Methyl Myristate, Methyl Nicotinate, Methyl 2-Octynoate, Methyl Oleate, Methyl Palmitate, Methylparaben, Methyl Pelargonate, Methyl Phenylbutanol, Methyl Rosinate, Methyl Salicylate, Methyl Stearate, MIBK, Michelia Alba Flower Oil, Michelia Alba Leaf Oil, Michelia Champaca Oil, Mimosa Tenuiflora Bark Extract, Mimosa Tenuiflora Leaf Extract, Mineral Oil, Mixed Cresols, Mixed lonones, Monarda Didyma Oil, Musa Sapientum (Banana) Flower Water, Musk Ketone, Myrcenol, Myrica Gale Extract, Myristic Acid, Myristica Fragrans (Nutmeg) Extract, Myristica Fragrans (Nutmeg) Kernel Oil, Myristyl Alcohol, Myrocarpus Fastigiatus Oil, Myroxylon Balsamum (Balsam Tolu) Resin, Myroxylon Pereirae (Balsam Peru) Oil, Myroxylon Pereirae (Balsam Peru) Resin, Myrrhis Odorata Extract, Myrtus Communis Leaf Water, Myrtus Communis Oil, 2-Naphthol, Narcissus Poeticus Extract, Narcissus Poeticus Flower Wax, Nasturtium Officinale Extract, Nelumbium Speciosum Flower Water, Nelumbo Nucifera Flower Water, Neohesperidin Dihydrochalcone, Nepeta Cataria Extract, Nicotiana Tabacum (Tobacco) Leaf Extract, Nindou Ekisu, Nitrous Oxide, Gamma-Nonalactone, Nonyl Acetate, Nopyl Acetate, Ocimum Basilicum (Basil) Extract, Ocimum Basilicum (Basil) Oil, Octadecane, Octyldodecanol, Olax Dissitiflora Root Oil, Olea Europaea (Olive) Fruit Oil, Oleic Acid, Oleth-2, Oleth-3, Oleth-4, Oleth-5, Oleth-6, Oleth-7, Oleth-8, Oleth-9, Oleth-10, Oleth-11, Oleth-12, Oleth-15, Oleth-16, Oleth-20, Oleth-23, Oleth-24, Oleth-25, Oleth-30, Oleth-35, Oleth-40, Oleth-44, Oleth-50, Oleth-82, Oleth-106, Oleyl Alcohol, Olibanum, Opoponax Oil, Orange Yu, Origanum Heracleoticum Flower Oil, Origanum Majorana Flower Oil, Origanum Majorana Leaf Extract, Origanum Majorana Leaf Oil, Origanum Vulgare Flower Extract, Ormenis Multicaulis Extract, Ormenis Multicaulis Oil, Oryza Sativa (Rice) Bran Water, Osmanthus Fragrans Flower Extract, Palmitic Acid, Panax Ginseng Root Water, Pandanus Amaryllifolius Leaf Extract, Paraffin, PEG-2 Hydrogenated Castor Oil, PEG-5 Hydrogenated Castor Oil, PEG-6 Hydrogenated Castor Oil, PEG-7 Hydrogenated Castor Oil, PEG-10 Hydrogenated Castor Oil, PEG-16 Hydrogenated Castor Oil, PEG-20 Hydrogenated Castor Oil, PEG-25 Hydrogenated Castor Oil, PEG-30 Hydrogenated Castor Oil, PEG-35 Hydrogenated Castor Oil, PEG-40 Hydrogenated Castor Oil, PEG-45 Hydrogenated Castor Oil, PEG-50 Hydrogenated Castor Oil, PEG-54 Hydrogenated Castor Oil, PEG-55 Hydrogenated Castor Oil, PEG-60 Hydrogenated Castor Oil, PEG-80 Hydrogenated Castor Oil, PEG-100 Hydrogenated Castor Oil, PEG-200 Hydrogenated Castor Oil, PEG-10 Sorbitan Laurate, PEG-40 Sorbitan Laurate, PEG-44 Sorbitan Laurate, PEG-75 Sorbitan Laurate, PEG-80 Sorbitan Laurate, PEG-3 Sorbitan Oleate, PEG-6 Sorbitan Oleate, PEG-3 Sorbitan Stearate, PEG-4 Sorbitan Stearate, PEG-6 Sorbitan Stearate, PEG-40 Sorbitan Stearate, PEG-60 Sorbitan Stearate, Pelargonic Acid, Pelargonium Graveolens Extract, Pelargonium Graveolens Flower Oil, Pelargonium Graveolens Water, Pelargonium Graveolens Wax, Pentadecalactone, Pentadecyl Alcohol, Pentamethylheptenone, Perillaldehyde, Persea Gratissima (Avocado) Fruit Water, Phenethyl Acetate, Phenethyl Alcohol, Phenol, Phenoxyethanol, Phenylalanine, Phenyl Benzoate, Phenylisohexanol, Phenylmethylpentanal, o-Phenylphenol, Phenylpropanol, Phenyl Salicylate, Phosphoric Acid, Phytol, Picea Excelsa Leaf Oil, Pimenta Acris (Bay) Leaf Oil, Pimpinella Anisum (Anise) Fruit Extract, Pinus Palustris Oil, Pinus Palustris Tar Oil, Pinus Pumilio Oil, Pinus Strobus Cone Extract, Pinus Sylvestris Cone Extract, Pinus Sylvestris Cone Oil, Pinus Sylvestris Leaf Oil, Piper Betle Leaf Oil, Piper Nigrum (Black Pepper) Fruit Oil, Pistacia Lentiscus (Mastic) Gum, Pogostemon Cablin Oil, Polianthes Tuberosa Extract, Polysorbate 20, Polysorbate 21, Polysorbate 60, Polysorbate 61, Polysorbate 80, Polysorbate 81, Pongamol, Potassium Acetate, Potassium Sorbate, PPG-2-Buteth-2, PPG-2-Buteth-3, PPG-3-Buteth-5, PPG-4-Buteth-4, PPG-5-Buteth-5, PPG-5-Buteth-7, PPG-7-Buteth-4, PPG-7-Buteth-10, PPG-9-Buteth-12, PPG-10-Buteth-9, PPG-12-Buteth-12, PPG-12-Buteth-16, PPG-15-Buteth-20, PPG-17-Buteth-17, PPG-19-Buteth-19, PPG-20-Buteth-30, PPG-24-Buteth-27, PPG-26-Buteth-26, PPG-28-Buteth-35, PPG-30-Buteth-30, PPG-33-Buteth-45, PPG-36-Buteth-36, PPG-38-Buteth-37, PPG-2 Methyl Ether, Proline, Propionic Acid, Propyl Acetate, Propyl Alcohol, Propyl Benzoate, Propylene Glycol, Propylene Glycol Alginate, Propylene Glycol Butyl Ether, Propylene Glycol Stearate, Propyl Gallate, Propylparaben, Prunus Amygdalus Amara (Bitter Almond) Kernel Oil, Prunus Amygdalus Dulcis (Sweet Almond) Oil, Prunus Armeniaca (Apricot) Fruit Water, Prunus Armeniaca (Apricot) Kernel Oil, Prunus Cerasus (Bitter Cherry) Seed Oil, Prunus Serotina (Wild Cherry) Bark Extract, Prunus Serotina (Wild Cherry) Fruit Extract, Punica Granatum Bark Extract, Punica Granatum Extract, Pyrocatechol, Pyrogallol, Pyrus Cyclonia Seed Extract, Pyrus Malus (Apple) Fruit Water, Pyruvic Acid, Quillaja Saponaria Bark Extract, Quillaja Saponaria Root Extract, Quinine, Raspberry Ketone, Raspberryketone Glucoside, Resorcinol, Rhamnose, Rhododendron Chrysanthum Leaf Extract, Rhododendron Ferrugineum Extract, Ricinus Communis (Castor) Seed Oil, Rosa Canina Flower, Rosa Canina Flower Oil, Rosa Centifolia Flower Extract, Rosa Centifolia Flower Oil, Rosa Damascena Extract, Rosa Damascena Flower Oil, Rosa Damascena Flower Water, Rosa Damascena Flower Wax, Rosa Eglentaria Extract, Rosa Gallica Flower Oil, Rosa Moschata Oil, Rosa Multiflora Fruit Extract, Rose Flower Oil, Rosmarinus Officinalis (Rosemary) Flower Wax, Rosmarinus Officinalis (Rosemary) Leaf Extract, Rosmarinus Officinalis (Rosemary) Leaf Oil, Rosmarinus Officinalis (Rosemary) Leaf Water, Rosmarinus Officinalis (Rosemary) Water, Rubus Fruticosus (Blackberry) Fruit Extract, Rubus Fruticosus (Blackberry) Leaf Extract, Rubus Idaeus (Raspberry) Fruit Water, Rubus Idaeus (Raspberry) Leaf Wax, Ruta Graveolens (Rue) Oil, Saccharin, Salicylic Acid, Salvia Lavandulaefolia Oil, Salvia Officinalis (Sage) Leaf Water, Salvia Officinalis (Sage) Oil, Salvia Sclarea (Clary) Oil, Sambucus Nigra Oil, Sambucus Nigra Wax, Santalum Album (Sandalwood) Oil, Sassafras Officinale Root Oil, Satureia Hortensis Extract, Schinus Molle Oil, Sciareolide, Serine, Sesamum Indicum (Sesame) Seed Oil, Sisymbrium Trio Seed Oil, Beta-Sitosterol, Sodium Acetate, Sodium Benzoate, Sodium Citrate, Sodium Glutamate, Sodium Hexametaphosphate, Sodium Saccharin, Solanum Lycopersicum (Tomato) Fruit Water, Sorbic Acid, Sorbitan Oleate, Sorbitan Stearate, Sorbitol, Spartium Junceum Flower Extract, Stearic Acid, Stearyl Alcohol, Sterculia Urens Gum, Styrax Benzoin Gum, Styrax Benzoin Resin Extract, Succinic Acid, Sucrose Octaacetate, Synthetic Wax, Tagetes Minuta Flower Oil, Taimu Yu, Tanacetum Cinerariifolium (Pyrethrum) Flower Extract, Tannic Acid, Tarchonanthus Camphoratus Oil, Tar Oil, Tartaric Acid, Taurine, TBHQ, Terpineol, 4-Terpineol, Terpineol Acetate, Tetrahydrofurfuryl Acetate, Tetrahydrofurfuryl Alcohol, Tetramethyl Cyclopentene Butenol, Theobroma Cacao (Cocoa) Seed Butter, Theobromine, Thiamine HCI, Thiolactic Acid, Threonine, Thuja Occidentalis Leaf Oil, Thymol, Thymus Mastichina Oil, Thymus Vulgaris (Thyme) Extract, Thymus Vulgaris (Thyme) Leaf, Thymus Vulgaris (Thyme) Oil, Thymus Zygis Oil, Tilia Americana Flower Extract, Tilia Cordata Flower Water, Tilia Cordata Oil, Tocopherol, Torreya Californica (California Nutmeg) Oil, Triacetin, Tribenzoin, Tricalcium Phosphate, Tricaprin, Tricaprylin, Tricyclodecenyl Propionate, Tridecyl Alcohol, Triethanolamine, Triethyl Citrate, Triethylene Glycol, Triethylhexanoin, Trifolium Pratense (Clover) Flower Extract, Trigonella Foenum-Graecum Seed Extract, Trimethylamine Oxide, Trimethylhexanol, Tromethamine, Tryptophan, Turpentine, Tyrosine, Uikyo Yu, Gamma-Undecalactone, Undecanoic Acid, Undecyl Alcohol, Undecylenal, Undecylenic Acid, Undecylenyl Alcohol, Ursolic Acid, Valeriana Officinalis Root, Valine, Vanilla Planifolia Fruit, Vanilla Tahitensis Fruit, Vanillin, Vanillyl Butyl Ether, Verbena Officinalis Extract, Vetiveria Zizanoides Root Oil, Viburnum Prunifolium Extract, Viola Odorata Extract, Viola Odorata Leaf Extract, Viola Odorata Leaf Wax, Viola Odorata Oil, Viola Tricolor Water, Vitis Vinifera (Grape) Leaf Oil, Ximenia Americana Seed Oil, Xylene, Xylose, Yucca Aloifolia Extract, Yucca Filamentosa Extract, Yucca Schidigera Extract, Yucca Vera Extract, Yukari Yu, Zanthoxylum Acanthopodium Fruit Oil, Zanthoxylum Americanum Bark Extract, Zanthoxylum Piperitum Oil, Zea Mays (Corn) Oil, Zingiber Officinale (Ginger) Root Extract, Zingiber Officinale (Ginger) Root Oil, and Zingiber Officinale (Ginger) Water. The perfume or fragrance may be used in the cosmetic composition in an amount ranging from 0.0001% to 10% by weight. In certain embodiments, the perfume or fragrance is used in the composition in an amount ranging from 0.01% to 1% by weight. In certain embodiments, the perfume or fragrance is used in the composition in an amount ranging from 0.001% to 0.01% by weight. In certain embodiments, the perfume or fragrance is used in the composition in an amount ranging from 0.001% to 0.1% by weight. In certain embodiments, the composition does not include a perfume or fragrance.

### Oils/Emollients/Lubricants/Butters

The inventive cosmetic compositions may include an oil, emollient, lubricant, or butter. These terms are used interchangeably herein. Oils are used in cosmetic compositions to moisturize and/or nourish the hair. In certain embodiments, an oil is any fatty substance which is liquid at room temperature (25° C.). Exemplary oils, emollients, lubricants, and butters include silicone oils; phenylsilicones; silicone resins; silicone gums; mineral oils such as paraffin oil or vaseline oil; oils of animal origin such as perhydrosqualene, squalene, lanolin; oils of plant origin such as liquid triglycerides, e.g., sunflower oil, corn oil, soybean oil, rice oil, jojoba oil, babusscu oil, pumpkin oil, grapeseed oil, sesame oil, walnut oil, apricot oil, macadamia oil, avocado oil, sweet almond oil, lady's-smock oil, castor oil, triglycerides of caprylic/capric acids, olive oil, peanut oil, rapeseed oil, and coconut oil; synthetic oils such as purcellin oil, isoparaffins, linear and/or branched fatty alcohols and fatty acid esters, preferably guerbet alcohols having 6 to 18, preferably 8 to 10, carbon atoms; esters of linear (C₆-C₁₃) fatty acids with linear (C₆-C₂₀) fatty alcohols; esters of branched (C₆-C₁₃) carboxylic acids with linear (C₆-C₂₀) fatty alcohols, esters of linear (C₆-C₁₈) fatty acids with branched alcohols, especially 2-ethylhexanol; esters of linear and/or branched fatty acids with polyhydric alcohols (such as dimerdiol or trimerdiol, for example) and/or guerbet alcohols; triglycerides based on (C₆-C₁₀) fatty acids; esters such as dioctyl adipate, diisopropyl dimer dilinoleate; propylene glycols/dicaprylate or waxes such as beeswax, paraffin wax or microwaxes, alone or in combination with hydrophilic waxes, such as cetylstearyl alcohol, for example; fluorinated and perfluorinated oils; fluorinated silicone oils; mixtures of the aforementioned compounds. Exemplary plant derived oils include almond oil, apricot kernel oil, arnica oil, avocado oil, babusscu oil, black cumin seed oil, borage seed oil, castor oil, coconut oil, colza oil, corn oil, cotton seed oil, cowslip oil, evening primrose seed oil, grapeseed oil, hazelnut oil, hemp seed oil, jojoba oil, kukui nut oil, lady's-smock oil, linseed oil, macadamia nut oil, menhaden oil, neem seed oil, olive oil, palm oil, palm seed oil, peanut oil, pine oil, pomegranate seed oil, pumpkin seed oil, rape oil, rapeseed oil, rice oil, rice palm oil, rosehip seed oil, safflower oil, seabuckthorn oil, sesame oil, sesame seed oil, shea nut oil, soya oil, soybean oil, sunflower oil, tamanu oil, vitamin E oil, wheat germ oil, and walnut oil. Exemplary animal-derived oils include squalene, perhydrosqualene, and lanolin. Exemplary synthetic oils include purcellin oil, isoparafiins, linear or branched hydrocarbons, linear or branched fatty alcohols, linear or branched fatty acids, and linear or branched fatty acid esters. Exemplary mineral oils include paraffin oil and vaseline oil. In certain embodiments, the oil is a glyceryl ester which is primarily a fatty acid mono-, di-, or triglyceride modified by reaction with other alcohols, for example, acetylated castor oil, glyceryl stearate, glyceryl dioleate, glyceryl distearate, glyceryl trioctanoate, glyceryl distearate, glyceryl linoleate, glyceryl myristate, glyceryl isostearate, PEG castor oils, PEG glyceryl oleates, PEG glyceryl stearates, PEG glyceryl tallowates, etc. In certain embodiments, the oil is a fluorinated oil. Examples of fluorinated oils include fluoro guerbet esters or perfluoropolyethers. Suitable perfluoropolyethers are disclosed in U.S. Pat. Nos. 5,183,589, 4,803,067, and 5,183,588, all of which are hereby incorporated by herein reference. In certain embodiments, the oil is a sorbitan derivative. Exemplary sorbitan derivatives include PEG sorbitan beeswax, PEG sorbitan isostearate, PEG sorbitan lanolate, PEG sorbitan laurate, PEG sorbitan oleate, PEG sorbitan palmitate, PEG sorbitan stearate, polysorbates, sorbitan trioleates, sorbitan sesquioleates, sorbitan stearates, sorbitan tristearates, etc. These perfluoropolyethers are commercially available from Montefluos under the trademark Fomblin. In certain embodiments, the inventive composition does not include a Fomblin. The oil is used in the hair care composition in an amount ranging from about 1% to about 50% by weight. In certain embodiments, the oil is used in the cosmetic composition in an amount ranging from about 1% to about 20% by weight. In certain embodiments, the oil is used in the cosmetic composition in an amount ranging from about 1% to about 10% by weight.

### Thickeners/Viscosity Modifiers

The inventive cosmetic compositions may include a thickening agent or a viscosity modifier. The thickening agent may be a natural or synthetic thickening agent. In certain embodiments, the thickening agent is polymeric. In certain embodiments, the thickening agent is a polysaccharide. In certain embodiments, the thickening agent is a protein. In certain embodiments, the thickening agent is a low melting point wax. Examples of low melting point waxes include emulsifying wax, and fatty alcohols having the formula R—OH, wherein R is a straight or branched chain, unsaturated or unsaturated alkyl moiety having from about 4 to 35, more preferably about 6 to 22, carbon atoms. Examples of fatty alcohols include stearyl alcohol, cetearyl alcohol, behenyl alcohol, and the like. In certain embodiments, the thickening agent is a synthetic polymeric thickener. Examples of synthetic polymeric thickeners include polymers of acrylic acid, methacrylic acid and their simple esters, which may be co-polymerized with one or more organic groups such as ethoxylated or propoxylated polymeric moieties. Examples of such synthetic polymeric thickeners include acrylamides copolymer, acrylates/behenth-25 methacrylate copolymer, acrylates C10 30 alkyl acrylate crosspolymer, acrylates ceteth-20 itaconate copolymer, acrylates/steareth-50 acrylate copolymer, acrylates/stearyl methacrylate copolymer, acrylates/vinyl isodecanoate crosspolymer, or mixtures thereof. In certain embodiments, the thickening agent is a natural polymeric thickener. In certain embodiments, the thickening agent is a cellulosic thickener. Examples of cellulosic thickeners include cellulose gum as well as alkyl and hydroxylalkyl derivatives of celluloses and methyl or ethyl cellulose, such as hydroxyethylcellulose, hydroxypropylcellulose, hydroxyethyl ethylcellulose, hydroxybutyl cellulose, or mixtures thereof. Other specific exemplary viscosity modifying agents include carrageenan, quince mucilage, carboxyvinyl polymer, xanthane gum, Acrylates/Bis-Hydroxypropyl Dimethicone Crosspolymer, Ammonium Phosphatidyl Rapeseedate, C40-60 Acid, Citrus Aurantium Dulcis (Orange) Peel Extract, Cocamide Methyl MEA, Diallyloxyneohexyl Zirconium Tridecanoate, Dimethyldibenzylidene Sorbitol, Ethylhexyl AcrylateNP/Dimethicone Methacrylate Copolymer, Glycereth-7/IPDI Copolymer, Hydrogenated Didodecene, Hydrogenated Polydodecene, Hydrogenated Styrene/Isoprene Copolymer, Hydrogenated Tridodecene, Hydroxypropyl Dimethiconylpropyl Acrylates Copolymer, Isoprene/Pentadiene Copolymer, Lauryl Dimethicone PEG-15 Crosspolymer, Polianthes Tuberosa Extract, Polysilicone-16, Pyrus Malus (Apple) Fiber, Rosa Multiflora Flower Wax, Sodium Polyacrylate, Sodium Trimethylpentene/MA Copolymer, Alcohol, Alcohol Denat., Benzyl Alcohol, Butoxydiglycol, Butoxyethanol, Butylene Glycol, CD Alcohol 19, Ceteareth-22, C7-8 Isoparaffin, C8-9 Isoparaffin, C9-11 Isoparaffin, C9-13 Isoparaffin, C9-14 Isoparaffin, C10-11 Isoparaffin, C10-12 Isoparaffin, C11-14 Isoparaffin, Decane, Decene, Deodorized Kerosene, Diethylene Glycol, Dimethyl Ether, Dimethyl Isosorbide, Dimethyl Sulfone, Dipropylene Glycol, Dodecene, Ethoxydiglycol, Ethoxyethanol, Ethyl Perfluorobutyl Ether, Glycereth-7, Glycereth-12, Glycereth-20, Glycereth-26, Glycereth-31, Glycerin, Glycofurol, Glycol, Heptane, Hexadecene, Hexane, 1,2,6-Hexanetriol, Hexyl Alcohol, Hexylene Glycol, Isobutoxypropanol, Isopentane, Isopropyl Alcohol, Methoxydiglycol, Methoxyethanol, Methoxyethanol Acetate, Methoxyisopropanol, Methyl Hexyl Ether, Methyl Perfluorobutyl Ether, Octadecene, Octene, Pentane, Polyglyceryl Sorbitol, Propanediol, Propyl Alcohol, Propylene Carbonate, Propylene Glycol, SD Alcohol 1, SD Alcohol 3-A, SD Alcohol 3-B, SD Alcohol 3-C, SD Alcohol 23-A, SD Alcohol 23-F, SD Alcohol 23-H, SD Alcohol 27-B, SD Alcohol 30, SD Alcohol 31-A, SD Alcohol 36, SD Alcohol 37, SD Alcohol 38-B, SD Alcohol 38-C, SD Alcohol 38-D, SD Alcohol 38-F, SD Alcohol 39, SD Alcohol 39-A, SD Alcohol 39-B, SD Alcohol 39-C, SD Alcohol 39-D, SD Alcohol 40, SD Alcohol 40-A, SD Alcohol 40-B, SD Alcohol 40-C, SD Alcohol 46, Sorbeth-6, Sorbeth-20, Sorbeth-30, Sorbeth-40, Tetradecene, Triethylene Glycol, Turpentine, Acetamide MEA, Acrylamides Copolymer, Acrylamide/Sodium Acrylate Copolymer, Acrylamide/Sodium Acryloyldimethyltaurate Copolymer, Acrylates/Acetoacetoxyethyl Methacrylate Copolymer, Acrylates/Beheneth-25 Methacrylate Copolymer, Acrylates/C10-30 Alkyl Acrylate Crosspolymer, Acrylates/Ceteth-20 Itaconate Copolymer, Acrylates/Ceteth-20 Methacrylate Copolymer, Acrylates/Laureth-25 Methacrylate Copolymer, Acrylates/Palmeth-25 Acrylate Copolymer, Acrylates/Palmeth-25 Itaconate Copolymer, Acrylates/Steareth-50 Acrylate Copolymer, Acrylates/Steareth-20 Itaconate Copolymer, Acrylates/Steareth-20 Methacrylate Copolymer, Acrylates/Stearyl Methacrylate Copolymer, Acrylates/Vinyl Isodecanoate Crosspolymer, Acrylic Acid/Acrylonitrogens Copolymer, Adipic Acid/Methyl DEA Crosspolymer, Agar, Agarose, Alcaligenes Polysaccharides, Algin, Alginic Acid, Almondamide DEA, Almondamidopropyl Betaine, Aluminum/Magnesium Hydroxide Stearate, Ammonium Acrylates/Acrylonitrogens Copolymer, Ammonium Acrylates Copolymer, Ammonium Acryloyldimethyltaurate/Vinyl Formamide Copolymer, Ammonium Acryloyldimethyltaurate/VP Copolymer, Ammonium Alginate, Ammonium Chloride, Ammonium Polyacryloyldimethyl Taurate, Ammonium Sulfate, Amylopectin, Apricotamide DEA, Apricotamidopropyl Betaine, Arachidyl Alcohol, Arachidyl Glycol, Arachis Hypogaea (Peanut) Flour, Ascorbyl Methylsilanol Pectinate, Astragalus Gummifer Gum, Attapulgite, Avena Sativa (Oat) Kernel Flour, Avocadamide DEA, Avocadamidopropyl Betaine, Azelamide MEA, Babassuamide DEA, Babassuamide MEA, Babassuamidopropyl Betaine, Behenamide DEA, Behenamide MEA, Behenamidopropyl Betaine, Behenyl Betaine, Bentonite, Butoxy Chitosan, Caesalpinia Spinosa Gum, Calcium Alginate, Calcium Carboxymethyl Cellulose, Calcium Carrageenan, Calcium Chloride, Calcium Potassium Carbomer, Calcium Starch Octenylsuccinate, C20-40 Alkyl Stearate, Canolamidopropyl Betaine, Capramide DEA, Capryl/Capramidopropyl Betaine, Carbomer, Carboxybutyl Chitosan, Carboxymethyl Cellulose Acetate Butyrate, Carboxymethyl Chitin, Carboxymethyl Chitosan, Carboxymethyl Dextran, Carboxymethyl Hydroxyethylcellulose, Carboxymethyl Hydroxypropyl Guar, Carnitine, Cellulose Acetate Propionate Carboxylate, Cellulose Gum, Ceratonia Siliqua Gum, Cetearyl Alcohol, Cetyl Alcohol, Cetyl Babassuate, Cetyl Betaine, Cetyl Glycol, Cetyl Hydroxyethylcellulose, Chimyl Alcohol, Cholesterol/HDI/Pullulan Copolymer, Cholesteryl Hexyl Dicarbamate Pullulan, Citrus Aurantium Dulcis (Orange) Peel Extract, Cocamide DEA, Cocamide MEA, Cocamide MIPA, Cocamidoethyl Betaine, Cocamidopropyl Betaine, Cocamidopropyl Hydroxysultaine, Coco-Betaine, Coco-Hydroxysultaine, Coconut Alcohol, Coco/Oleamidopropyl Betaine, Coco-Sultaine, Cocoyl Sarcosinamide DEA, Cornamide/Cocamide DEA, Cornamide DEA, Croscarmellose, Cyamopsis Tetragonoloba (Guar) Gum, Decyl Alcohol, Decyl Betaine, Dehydroxanthan Gum, Dextrin, Dibenzylidene Sorbitol, Diethanolaminooleamide DEA, DiglycoVCHDM/Isophthalates/SIP Copolymer, Dihydroabietyl Behenate, Dihydrogenated Tallow Benzylmonium Hectorite, Dihydroxyaluminum Aminoacetate, Dimethicone/PEG-15 Crosspolymer, Dimethicone Propyl PG-Betaine, DMAPA Acrylates/Acrylic Acid/Acrylonitrogens Copolymer, Erucamidopropyl Hydroxysultaine, Ethylene/Sodium Acrylate Copolymer, Gelatin, Gellan Gum, Glyceryl Alginate, Glycine Soja (Soybean) Flour, Guar Hydroxypropyltrimonium Chloride, Hectorite, Hyaluronic Acid, Hydrated Silica, Hydrogenated Potato Starch, Hydrogenated Tallow, Hydrogenated Tallowamide DEA, Hydrogenated Tallow Betaine, Hydroxybutyl Methylcellulose, Hydroxyethyl Acrylate/Sodium Acryloyldimethyl Taurate Copolymer, Hydroxyethylcellulose, Hydroxyethyl Chitosan, Hydroxyethyl Ethylcellulose, Hydroxyethyl Stearamide-MIPA, Hydroxylauryl/Hydroxymyristyl Betaine, Hydroxypropylcellulose, Hydroxypropyl Chitosan, Hydroxypropyl Ethylenediamine Carbomer, Hydroxypropyl Guar, Hydroxypropyl Methylcellulose, Hydroxypropyl Methylcellulose Stearoxy Ether, Hydroxypropyl Starch, Hydroxypropyl Starch Phosphate, Hydroxypropyl Xanthan Gum, Hydroxystearamide MEA, Isobutylene/Sodium Maleate Copolymer, Isostearamide DEA, Isostearamide MEA, Isostearamide MIPA, Isostearamidopropyl Betaine, Lactamide MEA, Lanolinamide DEA, Lauramide DEA, Lauramide MEA, Lauramide MIPA, Lauramide/Myristamide DEA, Lauramidopropyl Betaine, Lauramidopropyl Hydroxysultaine, Laurimino Bispropanediol, Lauryl Alcohol, Lauryl Betaine, Lauryl Hydroxysultaine, Lauryl Sultaine, Lecithinamide DEA, Linoleamide DEA, Linoleamide MEA, Linoleamide MIPA, Lithium Magnesium Silicate, Lithium Magnesium Sodium Silicate, Macrocystis Pyrifera (Kelp), Magnesium Alginate, Magnesium/Aluminum/Hydroxide/Carbonate, Magnesium Aluminum Silicate, Magnesium Silicate, Magnesium Trisilicate, Methoxy PEG-22/Dodecyl Glycol Copolymer, Methylcellulose, Methyl Ethylcellulose, Methyl Hydroxyethylcellulose, Microcrystalline Cellulose, Milkamidopropyl Betaine, Minkamide DEA, Minkamidopropyl Betaine, MIPA-Myristate, Montmorillonite, Moroccan Lava Clay, Myristamide DEA, Myristamide MEA, Myristamide MIPA, Myristamidopropyl Betaine, Myristamidopropyl Hydroxysultaine, Myristyl Alcohol, Myristyl Betaine, Natto Gum, Nonoxynyl Hydroxyethylcellulose, Oatamide MEA, Oatamidopropyl Betaine, Octacosanyl Glycol Isostearate, Octadecene/MA Copolymer, Oleamide DEA, Oleamide MEA, Oleamide MIPA, Oleamidopropyl Betaine, Oleamidopropyl Hydroxysultaine, Oleyl Betaine, Olivamide DEA, Olivamidopropyl Betaine, Oliveamide MEA, Palmamide DEA, Palmamide MEA, Palmamide MIPA, Palmamidopropyl Betaine, Palmitamide DEA, Palmitamide MEA, Palmitamidopropyl Betaine, Palm Kernel Alcohol, Palm Kernelamide DEA, Palm Kernelamide MEA, Palm Kernelamide MIPA, Palm Kernelamidopropyl Betaine, Peanutamide MEA, Peanutamide MIPA, Pectin, PEG-800, PEG-Crosspolymer, PEG-150/Decyl Alcohol/SMDI Copolymer, PEG-175 Diisostearate, PEG-190 Distearate, PEG-15 Glyceryl Tristearate, PEG-140 Glyceryl Tristearate, PEG-240/HDI Copolymer Bis-Decyltetradeceth-20 Ether, PEG-100/IPDI Copolymer, PEG-180/Laureth-50/TMMG Copolymer, PEG-10/Lauryl Dimethicone Crosspolymer, PEG-15/Lauryl Dimethicone Crosspolymer, PEG-2M, PEG-5M, PEG-7M, PEG-9M, PEG-14M, PEG-20M, PEG-23M, PEG-25M, PEG-45M, PEG-65M, PEG-90M, PEG-115M, PEG-160M, PEG-120 Methyl Glucose Trioleate, PEG-180/Octoxynol-40/TMMG Copolymer, PEG-150 Pentaerythrityl Tetrastearate, PEG-4 Rapeseedamide, PEG-150/Stearyl Alcohol/SMDI Copolymer, Phaseolus Angularis Seed Powder, Polianthes Tuberosa Extract, Polyacrylate-3, Polyacrylic Acid, Polycyclopentadiene, Polyether-1, Polyethylene/Isopropyl Maleate/MA Copolyol, Polyglyceryl-3 Disiloxane Dimethicone, Polyglyceryl-3 Polydimethylsiloxyethyl Dimethicone, Polymethacrylic Acid, Polyquatemium-52, Polyvinyl Alcohol, Potassium Alginate, Potassium Aluminum Polyacrylate, Potassium Carbomer, Potassium Carrageenan, Potassium Chloride, Potassium Palmate, Potassium Polyacrylate, Potassium Sulfate, Potato Starch Modified, PPG-2 Cocamide, PPG-1 Hydroxyethyl Caprylamide, PPG-2 Hydroxyethyl Cocamide, PPG-2 Hydroxyethyl Coco/Isostearamide, PPG-3 Hydroxyethyl Soyamide, PPG-14 Laureth-60 Hexyl Dicarbamate, PPG-14 Laureth-60 Isophoryl Dicarbamate, PPG-14 Palmeth-60 Hexyl Dicarbamate, Propylene Glycol Alginate, PVP/Decene Copolymer, PVP Montmorillonite, Pyrus Cyclonia Seed, Pyrus Malus (Apple) Fiber, Rhizobian Gum, Ricebranamide DEA, Ricinoleamide DEA, Ricinoleamide MEA, Ricinoleamide MIPA, Ricinoleamidopropyl Betaine, Ricinoleic Acid/Adipic Acid/AEEA Copolymer, Rosa Multiflora Flower Wax, Sclerotium Gum, Sesamide DEA, Sesamidopropyl Betaine, Sodium Acrylate/Acryloyldimethyl Taurate Copolymer, Sodium Acrylates/Acrolein Copolymer, Sodium Acrylates/Acrylonitrogens Copolymer, Sodium Acrylates Copolymer, Sodium Acrylates Crosspolymer, Sodium Acrylates/Vinyl Isodecanoate Crosspolymer, Sodium Acrylate/Vinyl Alcohol Copolymer, Sodium Carbomer, Sodium Carboxymethyl Chitin, Sodium Carboxymethyl Dextran, Sodium Carboxymethyl Beta-Glucan, Sodium Carboxymethyl Starch, Sodium Carrageenan, Sodium Cellulose Sulfate, Sodium Chloride, Sodium Cyclodextrin Sulfate, Sodium Hydroxypropyl Starch Phosphate, Sodium Isooctylene/MA Copolymer, Sodium Magnesium Fluorosilicate, Sodium Oleate, Sodium Palmitate, Sodium Palm Kernelate, Sodium Polyacrylate, Sodium Polyacrylate Starch, Sodium Polyacryloyldimethyl Taurate, Sodium Polygamma-Glutamate, Sodium Polymethacrylate, Sodium Polystyrene Sulfonate, Sodium Silicoaluminate, Sodium Starch Octenylsuccinate, Sodium Stearate, Sodium Stearoxy PG-Hydroxyethylcellulose Sulfonate, Sodium Styrene/Acrylates Copolymer, Sodium Sulfate, Sodium Tallowate, Sodium Tauride Acrylates/Acrylic Acid/Acrylonitrogens Copolymer, Sodium Tocopheryl Phosphate, Solanum Tuberosum (Potato) Starch, Soyamide DEA, Soyamidopropyl Betaine, Starch/Acrylates/Acrylamide Copolymer, Starch Hydroxypropyltrimonium Chloride, Stearamide AMP, Stearamide DEA, Stearamide DEA-Distearate, Stearamide DIBA-Stearate, Stearamide MEA, Stearamide MEA-Stearate, Stearamide MIPA, Stearamidopropyl Betaine, Steareth-60 Cetyl Ether, Steareth-100/PEG-136/HDI Copolymer, Stearyl Alcohol, Stearyl Betaine, Sterculia Urens Gum, Synthetic Fluorphlogopite, Tallamide DEA, Tallow Alcohol, Tallowamide DEA, Tallowamide MEA, Tallowamidopropyl Betaine, Tallowamidopropyl Hydroxysultaine, Tallowamine Oxide, Tallow Betaine, Tallow Dihydroxyethyl Betaine, Tamarindus Indica Seed Gum, Tapioca Starch, TEA-Alginate, TEA-Carbomer, TEA-Hydrochloride, Trideceth-2 Carboxamide MEA, Tridecyl Alcohol, Triethylene Glycol Dibenzoate, Trimethyl Pentanol Hydroxyethyl Ether, Triticum Vulgare (Wheat) Germ Powder, Triticum Vulgare (Wheat) Kernel Flour, Triticum Vulgare (Wheat) Starch, Tromethamine Acrylates/Acrylonitrogens Copolymer, Tromethamine Magnesium Aluminum Silicate, Undecyl Alcohol, Undecylenamide DEA, Undecylenamide MEA, Undecylenamidopropyl Betaine, Welan Gum, Wheat Germamide DEA, Wheat Germamidopropyl Betaine, Xanthan Gum, Yeast Beta-Glucan, Yeast Polysaccharides, Zea Mays (Corn) Starch, Abietyl Alcohol, Acrylates/C10-30 Alkyl Acrylate Crosspolymer, Adipic Acid/PPG-10 Copolymer, Allyl Methacrylates Crosspolymer, Alumina Magnesium Metasilicate, Aluminum Behenate, Aluminum Benzoate, Aluminum Caprylate, Aluminum Dilinoleate, Aluminum Dimyristate, Aluminum Distearate, Aluminum Isostearate, Aluminum Isostearates/Laurates/Palmitates, Aluminum Isostearates/Laurates/Stearates, Aluminum Isostearates/Myristates, Aluminum Isostearates/Palmitates, Aluminum Isostearates/Stearates, Aluminum Lanolate, Aluminum/Magnesium Hydroxide Stearate, Aluminum Myristate, Aluminum Myristates/Palmitates, Aluminum Starch Octenylsuccinate, Aluminum Stearate, Aluminum Stearates, Aluminum Tristearate, Arachidyl Alcohol, Arachidyl Behenate, Arachidyl Glycol, Beeswax, Behenamide, Behenamidopropyl Dimethylamine Behenate, Behenyl Alcohol, Behenyl Methacrylate/Perfluorooctylethyl, Methacrytate Copolymer, Bispolyethylene Dimethicone, Butadiene/Acrylonitrile Copolymer, Butylene/Ethylene Copolymer, Butylene/Ethylene/Styrene Copolymer, Butylene Glycol Cocoate, Butylene Glycol Montanate, Butyrospermum Parkii (Shea Butter), C29-70 Acid, C23-43 Acid Pentaerythritol Tetraester, C8-12 Acid Triglyceride, C12-18 Acid Triglyceride, Calcium Behenate, Calcium Laurate, Calcium Montanate, Calcium Myristate, Calcium Stearate, Calcium Undecylenate, C9-11 Alcohols, C12-13 Alcohols, C12-15 Alcohols, C12-16 Alcohols, C14-15 Alcohols, C20-22 Alcohols, C30-50 Alcohols, C40-60 Alcohols, C18-38 Alkyl C24-54 Acid Ester, C20-24 Alkyl Dimethicone, C24-28 Alkyl Dimethicone, C8-10 Alkyl Ethyl Phosphate, C1-5 Alkyl Galactomannan, C18-38 Alkyl Hydroxystearoyl Stearate, C20-24 Alkyl Methicone, C24-28 Alkyl Methicone, C30-45 Alkyl Methicone, Candelilla Wax Hydrocarbons, Carnauba Acid Wax, C10-30 Cholesterol/Lanosterol Esters, Cellobiose Octanonanoate, Ceresin, Cerotic Acid, Cetearyl Alcohol, Cetearyl Dimethicone/Vinyl Dimethicone Crosspolymer, Cetyl Alcohol, Cetyl Glycol, Chimyl Alcohol, Chlorinated Paraffin, Cholesterol, Cholesteryl Acetate, Cholesteryl Hydroxystearate, Cholesteryl Isostearate, Cholesteryl Macadamiate, Cholesteryl Stearate, C10-40 Hydroxyalkyl Acid Cholesterol Esters, C10-40 Isoalkyl Acid Cholesterol Esters, C10-40 Isoalkyl Acid Octyldodecanol Esters, C10-40 Isoalkyl Acid Phytosterol Esters, C10-40 Isoalkyl Acid Triglyceride, Coconut Alcohol, C30-38 Olefin/Isopropyl Maleate/MA Copolymer, Copal, Corn Starch Modified, C6-14 Perfluoroalkylethyl Acrylate/HEMA Copolymer, C6-14 Polyolefin, Cyclocarboxypropyloleic Acid, Decene/Butene Copolymer, Decyl Alcohol, 7-Dehydrocholesterol, Dibehenyl Fumarate, Dibenzylidene Sorbitol, Dihydrogenated Tallow Benzylmonium Hectorite, Dilinoleic Acid/Ethylenediamine Copolymer, Dilinoleic Acid/Sebacic Acid/piperazine/Ethylenediamine Copolymer, Dimer Dilinoleyl Diisostearate, Dimer Dilinoleyl Dimer Dilinoleate, Dimer Dilinoleyl Hydrogenated Rosinate, Dimethicone Crosspolymer, Dimethicone/Phenyl Vinyl Dimethicone Crosspolymer, Dimethicone/Polyglycerin-3 Crosspolymer, Dimethicone/Vinyl Dimethicone Crosspolymer, Dimethicone/Vinyltrimethylsiloxysilicate Crosspolymer, Dimethyldibenzylidene Sorbitol, Dipentaerythrityl Hexacaprylate/Hexacaprate, Dipentaerythrityl Hexaheptanoate/Hexacaprylate/Hexacaprate, Dipentaerythrityl Hexahydroxystearate, Dipentaerythrityl Hexahydroxystearate/Hexastearate/Hexarosinate, Dipentaerythrityl Hexaoctanoate/Hexabehenate, Dipentaerythrityl Pentahydroxystearate/Pentaisostearate, Dipentaerythrityl Tetrahydroxystearate/Tetraisostearate, Diphenyl Dimethicone/Vinyl Diphenyl Dimethicone/Silsesquioxane Crosspolymer, Divinyldimethicone/Dimethicone Crosspolymer, Dodecanedioic Acid/Cetearyl Alcohol/Glycol Copolymer, Ericerus Pela Wax, Erucamide, Ethylcellulose, Ethylene/Acrylic Acid Copolymer, Ethylene/Acrylic AcidNA Copolymer, Ethylenediamine/Dimer Tallate Copolymer Bis-Hydrogenated Tallow Amide, Ethylenediamine/Stearyl Dimer Dilinoleate Copolymer, Ethylenediamine/Stearyl Dimer Tallate Copolymer, Ethylene Dihydrogenated Tallowamide, Ethylene Dioleamide, Ethylene Distearamide, Ethylene/Octene Copolymer, Ethylene/Propylene Copolymer, Ethylene/Propylene/Styrene Copolymer, Ethylhexyl C10-40 Isoalkyl Acidate, Euphorbia Cerifera (Candelilla) Wax, Glyceryl Abietate, Glyceryl Arachidate, Glyceryl Diisostearate/Hydrogenated Rosinate, Glyceryl Stearate Diacetate, Glycol/Butylene Glycol Montanate, Glycol Dibehenate, Glycol Diethylhexanoate, Glycol Dilaurate, Glycol Dioleate, Glycol Distearate, Hexacosyl Glycol, Hexadecyleicosanoic Acid, Hexanediol Distearate, Hexyldecyloctadecanol, Hydroabietyl Alcohol, Hydrogenated Apricot Kernel Oil, Hydrogenated Butylene/Ethylene/Styrene Copolymer, Hydrogenated Canola Oil, Hydrogenated Castor Oil, Hydrogenated Castor Oil Hydroxystearate, Hydrogenated Castor Oil Isostearate, Hydrogenated Castor Oil Laurate, Hydrogenated Castor Oil Stearate, Hydrogenated Castor Oil Triisostearin Esters, Hydrogenated C6-14 Olefin Polymers, Hydrogenated Cottonseed Oil, Hydrogenated C12-18 Triglycerides, Hydrogenated Ethylene/Propylene/Styrene Copolymer, Hydrogenated Fish Oil, Hydrogenated Grapeseed Oil, Hydrogenated Japan Wax, Hydrogenated Lard, Hydrogenated Lard Glyceride, Hydrogenated Lard Glycerides, Hydrogenated Menhaden Oil, Hydrogenated Methyl Abietate, Hydrogenated Microcrystalline Wax, Hydrogenated Olive Oil, Hydrogenated Palm Glycerides, Hydrogenated Palm Kernel Glycerides, Hydrogenated Palm Kernel Oil, Hydrogenated Palm Oil, Hydrogenated Peanut Oil, Hydrogenated Pistachio Seed Oil, Hydrogenated Polyisobutene, Hydrogenated Rapeseed Oil, Hydrogenated Rice Bran Wax, Hydrogenated Rosin, Hydrogenated Safflower Seed Oil, Hydrogenated Shea Butter, Hydrogenated Soybean Oil, Hydrogenated Soy Glycerides, Hydrogenated Styrene/Butadiene Copolymer, Hydrogenated Styrene/Methyl Styrene/Indene Copolymer, Hydrogenated Sunflower Seed Oil, Hydrogenated Sweet Almond Oil, Hydrogenated Tallow Alcohol, Hydrogenated Tallow Amide, Hydrogenated Tallow Glycerides, Hydrogenated Vegetable Glycerides, Hydrogenated Vegetable Oil, Hydrogenated Wheat Germ Oil, Hydroxyoctacosanyl Hydroxystearate, Hydroxypropylcellulose, Isobutylene/Isoprene Copolymer, Isocetyl Alcohol, Isocetyl Stearoyl Stearate, Isostearyl Alcohol, Isostearyl Stearoyl Stearate, Jojoba Alcohol, Lanolin Alcohol, Lanolin Wax, Lard Glyceride, Lard Glycerides, Lauryl Alcohol, Lauryl Dimethicone/Polyglycerin-3 Crosspolymer, Linoleamide, Lithium Oxidized Polyethylene, Lithium Stearate, Magnesium Cocoate, Magnesium Lanolate, Magnesium Myristate, Magnesium Palmitate, Magnesium Stearate, Magnesium Tallowate, Mellisic Acid, Methoxy PEG-17/Dodecyl Glycol Copolymer, Methoxy PEG-22/Dodecyl Glycol Copolymer, Methyl Dehydroabietate, Methyl Dihydroabietate, Methyl Hydrogenated Rosinate, Methyl Methacrylate Crosspolymer, Methyl Rosinate, Methylstyrene/Vinyltoluene Copolymer, Microcrystalline Wax, Montan Acid Wax, Montan Wax, Myrica Cerifera (Bayberry) Fruit Wax, Myricyl Alcohol, Myristyl Alcohol, Neopentyl Glycol Dicaprate, Neopentyl Glycol Dicaprylate/Dicaprate, Neopentyl Glycol Dicaprylate/Dipelargonate/Dicaprate, Neopentyl Glycol Diethylhexanoate, Neopentyl Glycol Diheptanoate, Neopentyl Glycol Diisostearate, Neopentyl Glycol Dilaurate, Nylon-611/Dimethicone Copolymer, Octacosanyl Glycol, Octadecene/MA Copolymer, Octyldodecyl Stearoyl Stearate, Oleic/Linoleic/Linolenic Polyglycerides, Oleostearine, Oleyl Alcohol, Oleyl Palmitamide, Ouricury Wax, Oxidized Beeswax, Oxidized Microcrystalline Wax, Oxidized Polyethylene, Oxidized Polypropylene, Ozokerite, Palm Alcohol, Palm Kernel Alcohol, Paraffin, PEG-18 Castor Oil Dioleate, PEG-10 Dimethicone Crosspolymer, PEG-12 Dimethicone Crosspolymer, PEG-2 Dirosinate, PEG-3 Dirosinate, PEG-5 Hydrogenated Castor Oil Isostearate, PEG-10 Hydrogenated Castor Oil Isostearate, PEG-20 Hydrogenated Castor Oil Isostearate, PEG-30 Hydrogenated Castor Oil Isostearate, PEG-40 Hydrogenated Castor Oil Isostearate, PEG-50 Hydrogenated Castor Oil Isostearate, PEG-58 Hydrogenated Castor Oil Isostearate, PEG-50 Hydrogenated Castor Oil Succinate, PEG-5 Hydrogenated Castor Oil Triisostearate, PEG-10 Hydrogenated Castor Oil Triisostearate, PEG-15 Hydrogenated Castor Oil Triisostearate, PEG-20 Hydrogenated Castor Oil Triisostearate, PEG-30 Hydrogenated Castor Oil Triisostearate, PEG-40 Hydrogenated Castor Oil Triisostearate, PEG-60 Hydrogenated Castor Oil Triisostearate, PEG-5 Lanolinamide, PEG-5 Oleamide Dioleate, Pentaerythrityl Adipate/Caprate/Caprylate/Heptanoate, Pentaerythrityl Dioleate, Pentaerythrityl Distearate, Pentaerythrityl Hydrogenated Rosinate, Pentaerythrityl Isostearate/Caprate/Caprylate/Adipate, Pentaerythrityl Rosinate, Pentaerythrityl Stearate/Caprate/Caprylate/Adipate, Pentaerythrityl Stearate/Isostearate/Adipate/Hydroxystearate, Pentaerythrityl Tetraabietate, Pentaerythrityl Tetrabehenate, Pentaerythrityl Tetrabenzoate, Pentaerythrityl Tetracaprylate/Tetracaprate, Pentaerythrityl Tetracocoate, Pentaerythrityl Tetraethylhexanoate, Pentaerythrityl Tetraisononanoate, Pentaerythrityl Tetraisostearate, Pentaerythrityl Tetralaurate, Pentaerythrityl Tetramyristate, Pentaerythrityl Tetraoleate, Pentaerythrityl Tetrapelargonate, Pentaerythrityl Tetrastearate, Pentaerythrityl Trioleate, Phthalic Anhydride/Butyl Benzoic Acid/Propylene Glycol Copolymer, Phthalic Anhydride/Glycerin/Glycidyl Decanoate Copolymer, Phthalic Anhydride/Trimellitic Anhydride/Glycols Copolymer, Phytosteryl/Isostearyl/Cetyl/Stearyl/BehenylDimer Dilinoleate, Phytosteryl Isostearyl Dimer Dilinoleate, Piperylene/Butene/Pentene Copolymer, Polybutene, Polybutylene Terephthalate, Poly C10-30 Alkyl Acrylate, Polycyclopentadiene, Polydipentene, Polyethylene, Polyethylene Terephthalate, Polyglyceryl-3 Polyricinoleate, Polyglyceryl-4 Polyricinoleate, Polyglyceryl-5 Polyricinoleate, Polyglyceryl-10 Polyricinoleate, Polyisobutene, Polyisoprene, Polypentene, Polyperfluoroethoxymethoxy Difluoromethyl Distearamide, Polypropylene, Polysilicone-4, Polysilicone-5, Polysilicone-17, Polystyrene, Polyvinyl Butyral, Polyvinyl Laurate, Potassium Linoleate, Potassium Oxidized Microcrystalline Wax, Potassium Palm Kernelate, Potassium PEG-50 Hydrogenated Castor Oil Succinate, Potassium Rapeseedate, Potassium Soyate, Propylene Glycol Dicaprate, Propylene Glycol Dicaproate, Propylene Glycol Dicaprylate, Propylene Glycol Dicocoate, Propylene Glycol Diisononanoate, Propylene Glycol Diisostearate, Propylene Glycol Dilaurate, Propylene Glycol Dioleate, Propylene Glycol Dipelargonate, Propylene Glycol Distearate, Propylene Glycol Diundecanoate, Prunus Amygdalus Dulcis (Sweet Almond) Oil Unsaponifiables, PVM/MA Decadiene Crosspolymer, PVP/Decene Copolymer, Rhus Succedanea Fruit Wax, Rosa Multiflora Flower Wax, Rosin, Silica Dimethicone Silylate, Silica Dimethyl Silylate, Simmondsia Chinensis (Jojoba) Seed Wax, Sodium Acrylate/Sodium Acryloyldimethyl Taurate Copolymer, Sodium Linoleate, Sodium Olivate, Sodium Palmate, Sodium Peanutate, Sodium PVM/MA/Decadiene Crosspolymer, Sodium Rapeseedate, Sodium Rosinate, Sodium Soyate, Spent Grain Wax, Stearamide, Stearamide DEA-Distearate, Stearamide DIBA-Stearate, Stearamide MEA-Stearate, Steareth-10 Allyl Ether/Acrylates Copolymer, Steareth-60 Cetyl Ether, Stearone, Stearoxymethicone/Dimethicone Copolymer, Stearyl Alcohol, Stearyl Erucamide, Stearyl Erucate, Stearyl Glycol, Stearyl Glycol Isostearate, Stearyl Linoleate, Stearyl Methacrylate/Perfluorooctylethyl Methacrylate Copolymer, Stearyl Stearate, Stearyl Stearoyl Stearate, Styrene/Methacrylamide/Acrylates Copolymer, Synthetic Beeswax, Synthetic Candelilla Wax, Synthetic Carnauba, Synthetic Japan Wax, Synthetic Wax, Tallow Alcohol, Tallow Amide, TDI Oxidized Microcrystalline Wax, TEA-Rosinate, Tetradecyleicosanoic Acid, Tetradecyleicosanol, Tetradecyloctadecanoic Acid, Tetradecyloctadecanol, Tourmaline, Triarachidin, Tricontanyl PVP, Tridecyl Alcohol, Trierucin, Triethylene Glycol Hydrogenated Rosinate, Trifluoropropyl Dimethicone/PEG-10 Crosspolymer, Trifluoropropyl Dimethicone, Trifluoropropyl Divinyldimethicone Crosspolymer, Trifluoropropyl Dimethicone/Vinyl Trifluoropropyl Dimethicone/Silsesquioxane Crosspolymer, Triheptanoin, Triheptylundecanoin, Trihydroxystearin, Triisononanoin, Triisopalmitin, Triisostearin, Triisostearyl Trilinoleate, Trilaurin, Trilinoleic Acid, Trilinolein, Trilinolenin, Trimethylpentanediol/Isophthalic Acid/Trimellitic Anhydride Copolymer, Trimethylsiloxysilicate/Dimethiconol Crosspolymer, Trimyristin, Triolein, Tripalmitin, Tripalmitolein, Triricinolein, Trisebacin, Tristearin, Undecyl Alcohol, Vinyl Dimethicone/Lauryl Dimethicone Crosspolymer, Vinyl Dimethicone/Methicone Silsesquioxane Crosspolymer, VP/Eicosene Copolymer, VP/Hexadecene Copolymer, Zinc Laurate, Zinc Myristate, Zinc Neodecanoate, Zinc Palmitate, Zinc Rosinate, and Zinc Stearate. As would be appreciated by one of skill in the art, certain ingredients found in other categories of cosmetically acceptable excipients described herein may be used as thickening agents in the inventive compositions. The thickening agent may be added to the inventive composition until the desired viscosity of the final composition is achieved. Typically the concentration of the thickening agent in the final composition is in the range from about 1% to about 50% by weight. In certain embodiments, the concentration of thickening agent is about 5% to about 50% by weight. In certain embodiments, the concentration of thickening agent is about 2% to about 40% by weight. In certain embodiments, the concentration of thickening agent is about 2% to about 30% by weight. In certain embodiments, the concentration of thickening agent is about 5% to about 30% by weight. In certain embodiments, the concentration of thickening agent is about 2% to about 25% by weight. In certain embodiments, the concentration of thickening agent in the final composition is about 5%, about 10%, about 15%, about 20%, about 25%, about 30%, about 35%, about 40%, about 45%, or about 50%.

### Polymers/Resins/Film Formers

Polymers, resins, or film-forming agents may be used in the inventive cosmetic compositions. In certain embodiments, however, such ingredients are excluded from the inventive cosmetic composition. Any non-toxic polymer, resin, or film-forming agent may be used in the inventive composition. Natural as well as synthetic polymers may be used in the inventive compositions. Exemplary classes of polymers that may be used in the inventive compositions include lactide-glycolide copolymers, polyglyconate, poly(arylates), poly(anhydrides), poly(hydroxy acids), polyesters, poly(ortho esters), poly(alkylene oxides), polycarbonates, poly(fumarates), poly(alkylene fumarates), poly(caprolactones), polyamides, polyesters, polyethers, polyureas, polyamines, polyamino acids, polyacetals, poly(orthoesters), poly(pyrolic acid), poly(glaxanone), poly(phosphazenes), poly(organophosphazene), polylactides, polyglycolides, poly(dioxanones), polyhydroxybutyrate, polyhydroxyvalyrate, poly(vinyl pyrrolidone), polycyanoacrylates, polyurethanes, polysaccharides (e.g., chitin, starches, celluloses), polystyrenes, poly(vinyl alcohol), polyamides, poly(tetrafluoroethylene), poly(ethylene vinyl acetate), polypropylenes, polyacrylates, polymethacrylates, polyethylenes, polypyrroles, polyanilines, polythiophenes, poly(ethylene oxide), and mixtures, blends, and co-polymers thereof.

Specific exemplary polymers useful in the inventive cosmetic compositions include poly(N-vinyl pyrrolidone), vinyl pyrrolidone/vinyl acetate copolymer, poly(vinyl acetate), acrylic ester polymers, polyacrylic acids, poly(vinyl imidazole), cellulose ethers, N-vinyl-2-pyrrolidinone/vinyl acetate copolymers, vinyl acetate/crotonic acid copolymers, acrylic/sulfonamide/formaldehyde condensates, methyl vinyl ether/maleic anhydride copolymers, condensates of cyclohexanone, linear polyesters, branched polyesters, shellac, alkyl vinyl ether/maleic anhydride half-ester resins, vinyl acetate/monobutyl maleate/isobornyl acrylate resins, maleic anhydride-alkyl vinyl ether copolymer, polyvinylpyrrolidone-vinyl acetate copolymer, vinylpyrrolidone-vinyl caprolactam-dimethylaminoethyl methacrylate terpolymer, vinyl acetate-mono-n-butyl maleate-isobornyl acrylate terpolymer, polyvinylpyrrolidone, a copolymer of polyvinyl pyrrolidone and polyvinyl alcohol, a copolymer of polyvinyl pyrrolidone and hexadecene, butylated polyvinyl pyrrolidone, a copolymer of polyvinyl pyrrolidone and dimethylaminoethylmethacrylate, a butyl ester of a polymethylvinylether and maleic anhydride copolymer, and an ethyl ester of polymethylvinylether and maleic anhydride copolymer.

Exemplary film-forming agents useful in the inventive compositions include Abies Balsamea (Balsam Canada) Resin, Acetylenediurea/Formaldehyde/Tosylamide Crosspolymer, Acrylamide/Ammonium Acrylate Copolymer, Acrylamides Copolymer Acrylamides/DMAPA Acrylates/Methoxy PEG Methacrylate Copolymer, Acrylamide/Sodium Acrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/Behenyl Methacrylate/Dimethicone Methacrylate Copolymer, Acrylates/Bis-Hydroxypropyl Dimethicone Crosspolymer, Acrylates/t-Butylacrylamide Copolymer, Acrylates/C12-22 Alkyl Methacrylate Copolymer, Acrylates Copolymer, Acrylates/Diacetoneacrylamide Copolymer, Acrylates/Dimethicone Copolymer, Acrylates/Dimethicone Methacrylate Copolymer, Acrylates/Dimethiconol Acrylate Copolymer, Acrylates/Dimethylaminoethyl Methacrylate Copolymer, Acrylates/Ethylhexyl Acrylate Copolymer, Acrylates/Ethylhexyl Acrylate/HEMA/Styrene Copolymer, Acrylates/Ethylhexyl Acrylate/Styrene Copolymer, Acrylates/Hydroxyesters Acrylates Copolymer, Acrylates/Hydroxyethyl Acrylate/Lauryl Acrylate Copolymer, Acrylates/Hydroxyethyl Acrylate/Methoxyethyl Acrylate Copolymer, Acrylates/Lauryl Acrylate/Stearyl Acrylate/Ethylamine Oxide Methacrylate Copolymer, Acrylates/Octylacrylamide Copolymer, Acrylates/Propyl Trimethicone Methacrylate Copolymer, Acrylates/Stearyl Acrylate/Dimethicone Methacrylate Copolymer, Acrylates/Stearyl Acrylate/Ethylamine Oxide Methacrylate Copolymer, Acrylates/TDI/Trimethylolpropane Copolymer, Acrylates/VA Copolymer, Acrylates/VA Crosspolymer, Acrylates/VP Copolymer, Acrylates/VP/Dimethylaminoethyl Methacrylate/Diacetone Acrylamide/Hydroxypropyl Acrylate Copolymer, Acrylic Acid/Acrylonitrogens Copolymer, Adipic Acid/CHDM/MA/Neopentyl Glycol/Trimellitic Anhydride Copolymer, Adipic Acid/Diethylene GlycolGlycerin Crosspolymer, Adipic Acid/Diethylenetriamine Copolymer; Adipic Acid/Dilinoleic Acid/Hexylene Glycol Copolymer, Adipic Acid/Dimethylaminohydroxypropyl Diethylenetriamine Copolymer, Adipic Acid/Epoxypropyl Diethylenetriamine Copolymer, Adipic Acid/Fumaric Acid/Phthalic Acid/Tricyclodecane Dimethanol Copolymer, Adipic Acid/Isophthalic Acid/Neopentyl Glycol/Trimethylolpropane Copolymer, Adipic Acid/Methyl DEA Crosspolymer, Adipic Acid/Neopentyl Glycol/Trimellitic Anhydride Copolymer, Adipic Acid/PPG-10 Copolymer, Albumen, Allyl StearateNA Copolymer, Aloe Barbadensis Leaf Polysaccharides, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium Acrylates/Acrylonitrogens Copolymer, Ammonium Acrylates Copolymer, Ammonium Acrylates/Ethylhexyl Acrylate Copolymer, Ammonium Alginate, Ammonium Polyacrylate, Ammonium Styrene/Acrylates Copolymer, Ammonium Styrene/Acrylates/Ethylhexyl Acrylate/Lauryl Acrylate Copolymer, Ammonium VA/Acrylates Copolymer, Ammonium VA/Crotonic Acid Copolymer, Amodimethicone/Silsesquioxane Copolymer, AMP-Acrylates/Allyl Methacrylate Copolymer, AMP-Acrylates/C1-18 Alkyl Acrylates/C1-8 Alkyl Acrylamide Copolymer, AMP-Acrylates Copolymer, AMP-Acrylates/Diacetoneacrylamide Copolymer, AMP-Acrylates/Dimethylaminoethylmethacrylate Copolymer, AMP-Acrylates/Ethylhexyl Acrylate Copolymer, AMPD-Acrylates/Diacetoneacrylamide Copolymer, Astragalus Gummifer Gum, Avena Sativa (Oat) Kernel Protein, Behenyl Methacrylate/Perfluorooctylethyl Methacrylate Copolymer, Benzoguanamine/Formaldehyde/Melamine Crosspolymer, Benzoic Acid/Phthalic Anhydride/Pentaerythritol/Neopentyl Glycol/Palmitic Acid Copolymer, Bis-Hydrogenated Tallow Amine Dilinoleic Acid/Ethylenediamine Copolymer, Bis-PEG-15 Dimethicone/IPDI Copolymer, Bis-PPG-15 Dimethicone/IPDI Copolymer, Bis-Stearyl Dimethicone, Brassica Campestris/Aleurites Fordi Oil Copolymer, Butadiene/Acrylonitrile Copolymer, 1,4-Butandiol/Succinic Acid/Adipic Acid/HDI Copolymer, Butoxy Chitosan, Butyl Acrylate Crosspolymer, Butyl Acrylate/Ethylhexyl Methacrylate Copolymer, Butyl Acrylate/Hydroxyethyl Methacrylate Copolymer, Butyl Acrylate/Hydroxypropyl Dimethicone Acrylate Copolymer, Butyl Acrylate/Styrene Copolymer, Butylated Polyoxymethylene Urea, Butylated PVP, Butyl Benzoic Acid/Phthalic Anhydride/Trimethylolethane Copolymer, Butylene/Ethylene/Propylene Copolymer, Butyl Ester of Ethylene/MA Copolymer, Butyl Ester of PVM/MA Copolymer, Butylethylpropanediol Dimer Dilinoleate, Butyl Methacrylate/DMAPA Acrylates/Vinylacetamide Crosspolymer, C23-43 Acid Pentaerythritol Tetraester, Calcium Carboxymethyl Cellulose, Calcium Carrageenan, Calcium Potassium Carbomer, Calcium/Sodium PVM/MA Copolymer, C5-6 Alkane/Cycloalkane/Terpene Copolymer, C30-45 Alkyl Cetearyl Dimethicone Crosspolymer, C30-45 Alkyl Dimethicone/Polycyclohexene Oxide Crosspolymer, C1-5 Alkyl Galactomannan, Candelilla Wax Hydrocarbons, Carboxybutyl Chitosan, Carboxymethyl Cellulose Acetate Butyrate, Carboxymethyl Chitosan, Carboxymethyl Chitosan Succinamide, Carboxymethyl Dextran, Carboxymethyl Hydroxyethylcellulose, Carnauba Acid Wax, Castor Oil/IPDI Copolymer, Cellulose Acetate, Cellulose Acetate Butyrate, Cellulose Acetate Propionate, Cellulose Acetate Propionate Carboxylate, Cellulose Gum, Cetearyl Dimethicone/Vinyl Dimethicone Crosspolymer, Chitosan, Chitosan Adipate, Chitosan Ascorbate, Chitosan Formate, Chitosan Glycolate, Chitosan Lactate, Chitosan PCA, Chitosan Salicylate, Chitosan Succinamide, C5-6 Olefin/C8-10 Naphtha Olefin Copolymer, Collodion, Copaifera Officinalis (Balsam Copaiba) Resin, Copal, Corn Starch/Acrylamide/Sodium Acrylate Copolymer, Corn Starch Modified, C6-14 Perfluoroalkylethyl Acrylate/HEMA Copolymer, Cyclodextrin Laurate, DEA-Styrene/Acrylates/DVB Copolymer, Dehydroxanthan Gum, Dibutylhexyl IPDI, Didecyltetradecyl IPDI, Diethylene Glycolamine/Epichlorohydrin/piperazine Copolymer, Diethylene Glycol Rosinate, Diethylhexyl IPDI, Diglycol/CHDM/Isophthalates/SIP Copolymer, Diglycol/Isophthalates/SIP Copolymer, Dihydroxyethyl Tallowamine/IPDI Copolymer, Dilinoleic Acid/Butanediol Copolymer, Dilinoleic Acid/Glycol Copolymer, Dilinoleic Acid/Sebacic Acid/piperazine/Ethylenediamine Copolymer, Dilinoleyl Alcohol/IPDI Copolymer, Dimethicone PEG-8 Polyacrylate, Dimethicone/Silsesquioxane Copolymer, Dimethicone/Vinyltrimethylsiloxysilicate Crosspolymer, Dimethiconol/IPDI Copolymer, Dimethylamine/Ethylenediamine/Epichlorohydrin Copolymer, Dioctyldecyl IPDI, Dioctyldodecyl IPDI, Di-PPG-3 Myristyl Ether Adipate, Divinyldimethicone/Dimethicone Copolymer, Divinyldimethicone/Dimethicone Crosspolymer, DMAPA Acrylates/Acrylic Acid/Acrylonitrogens Copolymer, Dodecanedioic Acid/Cetearyl Alcohol/Glycol Copolymer, Ethylcellulose, Ethylene/Acrylic Acid Copolymer, Ethylene/Acrylic Acid/VA Copolymer, Ethylene/Calcium Acrylate Copolymer, Ethylene/MA Copolymer, Ethylene/Magnesium Acrylate Copolymer, Ethylene/Methacrylate Copolymer, Ethylene/Octene Copolymer, Ethylene/Propylene Copolymer, Ethylene/Sodium Acrylate Copolymer, Ethylene/VA Copolymer, Ethylene/Zinc Acrylate Copolymer, Ethyl Ester of PVM/MA Copolymer, Euphorbia Cerifera (Candelilla) Wax, Euphorbia Cerifera (Candelilla) Wax Extract, Fibroin/PEG-16/Sodium Acrylate Copolymer, Flexible Collodion, Formaldehyde/Melamine/Tosylamide Copolymer, Galactoarabinan, Glycereth-7 Hydroxystearate/IPDI Copolymer, Glycerin/MA/Rosin Acid Copolymer, Glycerin/Phthalic Acid Copolymer, Glycerin/Phthalic Acid Copolymer Castorate, Glycerin/Succinic Acid Copolymer Castorate, Glyceryl Diricinoleate/IPDI Copolymer, Glyceryl Polyacrylate, Glyceryl Polymethacrylate, Glyceryl Undecyl Dimethicone, Glycidyl C8-11 Acidate/Glycerin/Phthalic Anhydride Copolymer, Glycol Rosinate, Glycosyl Trehalose, Gutta Percha, Hexylene Glycol/Neopentyl Glycol/Adipic Acid/SMDI/DMPA Copolymer, Hydrogenated Brassica Campestris/Aleurites Fordi Oil Copolymer, Hydrogenated Caprylyl Olive Esters, Hydrogenated Cetyl Olive Esters, Hydrogenated Decyl Olive Esters, Hydrogenated Hexyl Olive Esters, Hydrogenated Lauryl Olive Esters, Hydrogenated Myristyl Olive Esters, Hydrogenated Rosin, Hydrogenated Styrene/Butadiene Copolymer, Hydrolyzed Candelilla Wax, Hydrolyzed Carnauba Wax, Hydrolyzed Chitosan, Hydrolyzed Gadidae Protein, Hydrolyzed Jojoba Esters, Hydrolyzed Sunflower Seed Wax, Hydrolyzed Wheat Protein, Hydrolyzed Wheat Protein/Cystine Bis-PG-Propyl Silanetriol Copolymer, Hydrolyzed Wheat Protein/Dimethicone PEG-7 Acetate, Hydrolyzed Wheat Protein/Dimethicone PEG-7 Phosphate Copolymer, Hydrolyzed Wheat Protein/PVP Crosspolymer, Hydroxybutyl Methylcellulose, Hydroxyethyl Acrylate/Methoxyethyl Acrylate Copolymer, Hydroxyethylcellulose, Hydroxyethyl Chitosan, Hydroxyethyl Ethylcellulose, Hydroxyethyl/Methoxyethyl Acrylate/Butyl Acrylate Copolymer, Hydroxyethyl/Methoxyethyl Acrylate Copolymer, Hydroxypropylcellulose, Hydroxypropyl Chitosan, Hydroxypropyl Dimethiconylpropyl Acrylates Copolymer, Hydroxypropyl Guar, Hydroxypropyl Methylcellulose, Hydroxypropyl Methylcellulose Acetate/Succinate, Hydroxypropyl Oxidized Starch, Hydroxypropyltrimonium Hyaluronate, Hydroxypropyl Xanthan Gum, Isobutylene/Ethylmaleimide/Hydroxyethylmaleimide Copolymer, Isobutylene/MA Copolymer, Isobutylene/Sodium Maleate Copolymer, Isomerized Linoleic Acid, Isophorone Diamine/Cyclohexylamine/Isophthalic Acid/Azelaic Acid Copolymer, Isophoronediamine/Isophthalic Acid/Pentaerythritol Copolymer, Isophorone Diamine/Isophthalic Acid/Trimethylolpropane Copolymer, Isopropyl Ester of PVM/MA Copolymer 4,4′-Isopropylidenediphenol/Epichlorohydrin Copolymer, Isostearoyl Epoxy Resin, Lauryl Acrylate/VA Copolymer, Lauryl Methacrylate/Glycol Dimethacrylate Crosspolymer, Maltodextrin, Mannan, Melia Azadirachta Conditioned Media/Culture, Methacrylic Acid/Sodium Acrylamidomethyl Propane Sulfonate Copolymer, Methacryloyl Ethyl Betaine/Acrylates Copolymer Methacryloyl Propyltrimethoxysilane, Methoxy Amodimethicone/Silsesquioxane Copolymer, Methoxypolyoxymethylene Melamine, Methyl Ethylcellulose, Methyl Methacrylate/Acrylonitrile Copolymer, Methyl Methacrylate Crosspolymer, Methyl Methacrylate/Glycol Dimethacrylate Crosspolymer, Myrica Cerifera (Bayberry) Fruit Wax, Myroxylon Balsamum (Balsam Tolu) Resin, Myroxylon Pereirae (Balsam Peru) Resin, Nitrocellulose, Nylon-12/6/66 Copolymer, Octadecene/MA Copolymer, Octylacrylamide/AcrylatesButylaminoethyl Methacrylate Copolymer, Oleoyl Epoxy Resin, Oxymethylene/Melamine Copolymer, Palmitic Acid/Pentaerythritol/Stearic Acid/Terephthalic Acid Copolymer, PEG-150/Decyl Alcohol/SMDI Copolymer, PEG-7 Dimethicone, PEG-114 Methylether Polyepsilon Capralactone, PEG/PPG-25/25 Dimethicone/Acrylates Copolymer, PEG-150/Stearyl Alcohol/SMDI Copolymer, Pentaerythritol/Terephthalic Acid Copolymer, Pentaerythrityl Cyclohexane Dicarboxylate, Perfluorononylethyl Stearyl Dimethicone, Phenylpropyldimethylsiloxysilicate, Phthalic Acid Denatured With Epoxy Resin Alkyd Resin, Phthalic Anhydride/Adipic Acid/Castor Oil/Neopentyl GlycoVPEG-3/Trimethylolpropane Copolymer, Phthalic Anhydride/Benzoic Acid/Glycerin Copolymer, Phthalic Anhydride/Benzoic Acid/Trimethylolpropane Copolymer, Phthalic Anhydride/Butyl Benzoic Acid/Propylene Glycol Copolymer, Phthalic Anhydride/Glycerin/Glycidyl Decanoate Copolymer, Phthalic Anhydride/Trimellitic Anhydride/Glycol Copolymer, Piperylene/Butene/Pentene Copolymer, Piperylene/Butene/Pentene/Pentadiene Copolymer, Pistacia Lentiscus (Mastic) Gum, Polianthes Tuberosa Extract, Polyacrylamide, Polyacrylamidomethylpropane Sulfonic Acid, Polyacrylate-1, Polyacrylate-2, Polyacrylate-5, Polyacrylate-6, Polyacrylate-9, Polyacrylic Acid, Polyamide-1, Polybeta-Alanine, Polybeta-Alanine/Glutaric Acid Crosspolymer, Polybutyl Acrylate, Polybutylene Terephthalate, Polybutyl Methacrylate, Polychlorotrifluoroethylene, Polydiethyleneglycol Adipate/IPDI Copolymer, Polydimethylaminoethyl Methacrylate, Polyester-1, Polyester-2, Polyester-3, Polyethylacrylate, Polyethylene, Polyethylene Naphthalate, Polyethylene Terephthalate, Polyethylglutamate, Polyethylhexyl Acrylate, Polyethylhexyl Methacrylate, Polyethylmethacrylate, Polyglucuronic Acid, Polyglyceryl-2 Diisostearate/IPDI Copolymer, Polyimide-1, Polyisobutene, Polylysine, Polymethacrylamide, Polymethacrylamidopropyltrimonium Metho-sulfate, Polymethacrylic Acid, Polymethyl Acrylate, Polymethylglutamate, Polymethyl Methacrylate, Polyoxyisobutylene/Methylene Urea Copolymer, Polyoxymethylene Melamine, Polypentaerythrityl Terephthalate, Polypentene, Polyperfluoroperhydrophenanthrene, Poly-p-Phenylene Terephthalamide, Polyphosphorylcholine Glycol Acrylate, Polypropyl Methacrylate, Polypropylsilsesquioxane, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-43, Polyquaternium-44, Polyquaternium-45, Polyquaternium-46, Polyquaternium-47, Polyquaternium-48, Polyquaternium-49, Polyquaternium-50, Polyquaternium-51, Polyquaternium-56, Polyquaternium-57, Polyquaternium-61, Polyquaternium-62, Polyquaternium-4/Hydroxypropyl Starch Copolymer, Polysilicone-6, Polysilicone-8, Polysilicone-11, Polysilicone-14, Polystyrene, Polyurethane-1, Polyurethane-2, Polyurethane-4, Polyurethane-5, Polyurethane-6, Polyurethane-7, Polyurethane-8, Polyurethane-10, Polyurethane-11, Polyurethane-12, Polyurethane-13, Polyurethane-14, Polyurethane-15, Polyvinylacetal Diethylaminoacetate, Polyvinyl Acetate, Polyvinyl Alcohol, Polyvinyl Butyral, Polyvinylcaprolactam, Polyvinyl Chloride, Polyvinyl Imidazolinium Acetate, Polyvinyl Isobutyl Ether, Polyvinyl Laurate, Polyvinyl Methyl Ether, Polyvinyl Stearyl Ether, Potassium Acrylates/Acrylamide Copolymer, Potassium Acrylates/C10-30 Alkyl Acrylate Crosspolymer, Potassium Acrylates/Ethylhexyl Acrylate Copolymer, Potassium Butyl Ester of PVM/MA Copolymer, Potassium Carbomer, Potassium Carrageenan, Potassium Ethyl Ester of PVM/MA Copolymer, PPG-26/HDI Copolymer, PPG-17/IPDI/DMPA Copolymer, PPG-12/SMDI Copolymer, PPG-7/Succinic Acid Copolymer, PPG-261TD1 Copolymer, PPG-10 Tocophereth-30, PPG-20 Tocophereth-50, Propylene Glycol Diricinoleate/IPDI Copolymer, Propyl Methacrylate, Pseudotsuga Menziesii (Balsam Oregon) Resin, Pullulan, PVM/MA Copolymer, PVM/MA Decadiene Crosspolymer, PVP, PVP Montmorillonite, PVP/VA/Itaconic Acid Copolymer, PVP/VA/Vinyl Propionate Copolymer, Quatemium-22, Rhizobian Gum, Ricinoleoyl Epoxy Resin, Rosin, Rubber Latex, Serum Albumin, Shellac, Sodium Acrylates/Acrolein Copolymer, Sodium Acrylates/Acrylonitrogens Copolymer, Sodium Acrylates/C10-30 Alkyl Acrylates Crosspolymer, Sodium Acrylates Copolymer, Sodium Acrylates/Ethylhexyl Acrylate Copolymer, Sodium Acrylate/Vinyl Alcohol Copolymer, Sodium Butyl Ester of PVM/MA Copolymer, Sodium Carbomer, Sodium Carboxymethyl Chitin, Sodium Carboxymethyl Starch, Sodium Carrageenan, Sodium C4-12 Olefin/Maleic Acid Copolymer, Sodium DVB/Acrylates Copolymer, Sodium Ethyl Ester of PVM/MA Copolymer, Sodium Isooctylene/MA Copolymer, Sodium MA/Diisobutylene Copolymer, Sodium MA/Vinyl Alcohol Copolymer, Sodium PG-Propyldimethicone Thiosulfate Copolymer, Sodium Polyacrylate, Sodium Polymethacrylate, Sodium Polystyrene Sulfonate, Sodium PVM/MA/Decadiene Crosspolymer, Sodium Styrene/Acrylates Copolymer, Sodium Styrene/Acrylates/Ethylhexyl Acrylate/Lauryl Acrylate Copolymer, Sodium Tauride Acrylates/Acrylic Acid/Acrylonitrogens Copolymer, Starch/Acrylates/Acrylamide Copolymer, Starch Diethylaminoethyl Ether, Stearamidopropyl Dimethicone, Steareth-10 Allyl Ether/Acrylates Copolymer, Stearoyl Epoxy Resin, Stearyl HDI/PEG-50 Copolymer, Stearyl Methacrylate/Perfluorooctylethyl Methacrylate Copolymer, Stearylvinyl Ether/MA Copolymer, Styrax Benzoin Gum, Styrene/Acrylates/Acrylonitrile Copolymer, Styrene/Acrylates/Ammonium Methacrylate Copolymer, Styrene/Acrylates Copolymer, Styrene/Acrylates Copolymer/Polyurethane, Styrene/Acrylates/Ethylhexyl Acrylate/Lauryl Acrylate Copolymer, Styrene/Allyl Benzoate Copolymer, Styrene/DVB Crosspolymer, Styrene/Isoprene Copolymer, Styrene/MA Copolymer, Styrene/Methacrylamide/Acrylates Copolymer, Styrene/Methylstyrene/Indene Copolymer, Styrene/VA Copolymer, Styrene/VP Copolymer, Sucrose Benzoate/Sucrose Acetate Isobutyrate/Butyl Benzyl Phthalate Copolymer, Sucrose Benzoate/Sucrose Acetate Isobutyrate/Butyl Benzyl Phthalate/Methyl Methacrylate Copolymer, Sucrose Benzoate/Sucrose Acetate Isobutyrate Copolymer, TEA-Acrylates/Acrylonitrogens Copolymer, TEA-Acrylates/Ethylhexyl Acrylate Copolymer, TEA-Diricinoleate, TEA-Diricinoleate/IPDI Copolymer, Terephthalic Acid/Isophthalic Acid/Sodium Isophthalic Acid Sulfonate/Glycol Copolymer, Tetradecyloctadecyl Behenate, Tetradecyloctadecyl Myristate, Tetradecyloctadecyl Stearate, TIPA-Acrylates/Ethylhexyl Acrylate Copolymer, Titanium Isostearates, Tosylamide/Epoxy Resin, Tosylamide/Formaldehyde Resin, Tricontanyl PVP, Triethylene Glycol Rosinate, Trifluoroethylmethacrylate/Hydroxypropyl Dimethiconylpropyl Acrylates Copolymer, Trimethylol Propane Cyclohexene Dicarboxylate, Trimethylolpropane Triacrylate, Trimethylpentanediol/Isophthalic Acid/Trimellitic Anhydride Copolymer, Trimethylsiloxysilicate/Dimethiconol Crosspolymer, Trimethylsiloxysilylcarbamoyl Pullulan, Triticum Vulgare (Wheat) Protein, Tromethamine Acrylates/Acrylonitrogens Copolymer, VA/Butyl Maleate/Isobornyl Acrylate Copolymer, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/Crotonic Acid/PEG-20M Copolymer, VA/DBM Copolymer, VA/Isobutyl Maleate/Vinyl Neodecanoate Copolymer, VA/Vinyl Butyl Benzoate/Crotonates Copolymer, VA/Vinyl Chloride Copolymer, Vinyl Acetate, Vinylamine/Vinyl Alcohol Copolymer, Vinyl Caprolactam % VP/Dimethylaminoethyl Methacrylate Copolymer, Vinyl Chloride/Vinyl Laurate Copolymer, VP/Dimethiconylacrylate/Polycarbamyl/Polyglycol Ester, VP/Dimethylaminoethylmethacrylate Copolymer, VP/Dimethylaminoethylmethacrylate/Polycarbamyl Polyglycol Ester, VP/Eicosene Copolymer, VP/Hexadecene Copolymer, VP/MethacrylamideNinyl Imidazole Copolymer, VP/Polycarbamyl Polyglycol Ester, VP/VA Copolymer, Welan Gum, Yeast Beta-Glucan, Yeast Polysaccharides, and Zein.

The polymer, resin, or film-forming agent is used in the inventive cosmetic composition at a concentration to achieve the desired result when applied to skin. In certain embodiments, the polymer, resin, or film-forming agent is used in the final composition in a range from about 0.01% to about 20% by weight. In certain embodiments, the polymer, resin, or film-forming agent is used in the final composition in a range from about 0.1% to about 10% by weight. In certain embodiments, the polymer, resin, or film-forming agent is used in the final composition in a range from about 0.1% to about 5% by weight. In certain embodiments, the polymer, resin, or film-forming agent is used in the final composition in a range from about 1% to about 5% by weight.

### Surfactants/Detergents/Emulsifiers

Surfactants, detergents, emulsifiers, and the like may be used in the inventive cosmetic compositions. Such agents may work to make the final composition homogenous or help to solubilize certain ingredients of the composition. In certain embodiments, the surfactant is an anionic surfactant. In other embodiments, the surfactant is a cationic surfactant. In yet other embodiments, the surfactant is a nonionic surfactant. In certain embodiments, the surfactant is a zwitterionic surfactant. In certain embodiments, the surfact is an amphoteric surfactant.

The surfactant used in the inventive composition may be chosen based on its HLB (Hydrophile-Lipophile Balance) value. In certain embodiments, the surfactant has an average HLB of less than or equal to about 7. In certain embodiments, the surfactant has an average HLB of less than or equal to about 10. In certain embodiments, the surfactant has an average HLB of greater than or equal to about 10. In certain embodiments, the surfactant has an average HLB of ranging from about 10 to about 15. In certain embodiments, the surfactant has an average HLB of ranging from about 10 to about 12. In certain embodiments, the surfactant has an average HLB of ranging from about 12 to about 18. In certain embodiments, the surfactant has an average HLB of ranging from about 14 to about 16. The HLB System was introduced in the late 1940's by ICI Americas Inc. Methods of determining HLB are well known in the art and any of such methods may be used for HLB determination. A description of the HLB System and methods for HLB determination are described in “The HLB System: a time saving guide to emulsifier selection,” ICI Americas Inc., Wilmington, Del., 1976.

Exemplary surfactants useful in the present invention include sodium dioctyl sulfo succinate, sodium dodecyl sulfate, cocoamidopropyl betaine, and sodium laureth sulfate, alkyl and alkyl ether sulfates (e.g., sodium coconut alkyl triethylene glycol ether sulfate; lithium tallow alkyl triethylene glycol ether sulfate; sodium tallow alkyl hexaoxyethylene sulfate), succinamates, sulfosuccinamates (e.g., disodium N-octadecyl-sulfosuccinamate, tetrasodium N-(1,2-dicarboxyethyl)-N-octadecylsulfosuccinamate, diamyl ester of sodium sulfosuccinic acid, dihexyl ester of sodium sulfosuccinic acid, dioctyl esters of sodium sulfosuccinic acid), olefin sulfonates, hydroxy-alkanesulfonates, beta-alkyloxy alkane sulfonates (e.g., potassium-β-methoxydecanesulfonate, sodium 2-methoxytridecanesulfonate, potassium 2-ethoxytetradecylsulfonate, sodium 2-isopropoxyhexadecylsulfonate, lithium 2-t-butoxytetradecylsulfonate, sodium β-methoxyoctadecysulfonate, ammonium β-n-propoxy-dodecylsulfonate), dioctyl esters of sodium sulfosuccinic acid, alkyl ethoxylated sulfates, alkyl sulfates, aliphatic secondary and tertiary amines (e.g., sodium 3-dodecylaminopropionate, N-alkyltaurines, stearamido propyl dimethyl amine, diethyl amino ethyl stearamide, dimethyl stearamine, dimethyl soyamine, soyamine, myristyl amine, tridecyl amine, ethyl stearylamine, N-tallowpropane diamine, ethoxylated (5 moles E.O) stearylamine, dihydroxy ethyl stearylamine, and arachidylbehenylamine), alkyl amphoglycinates (e.g., cocoamphoglycinate, lauroamphocarboxyglycinate, cocoamphocarboxyglycinate); alkyl amphopropionates (e.g., isostearoamphopropionate, cocoamphocarboxypropionic acid); alkyl ethoxylated sulfates; alkyl sulfates; aliphatic quaternary ammonium compounds (e.g., tallow propane diammonium dichloride, dialkyldimethylammonium chlorides, ditallowedimethyl ammonium chloride, ditallowedimethyl ammonium methyl sulfate, dihexadecyl dimethyl ammonium chloride, di(hydrogenated tallow) dimethyl ammonium chloride, dioctadecyl dimethyl ammonium chloride, dieicosyl dimethyl ammonium chloride, didocosyl dimethyl ammonium chloride, di(hydrogenated tallow) dimethyl ammonium acetate, dihexadecyl dimethyl ammonium chloride, dihexadecyl dimethyl ammonium acetate, ditallow dipropyl ammonium phosphate, ditallow dimethyl ammonium nitrate, and di(coconutalkyl benzyl ammonium chloride); aliphatic phosphonium compounds, aliphatic sulfonium compounds, alkyl amino sulfonates, alkyl betaines (e.g., coco dimethyl carboxymethyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxy methyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine), sulfo betaines (e.g., coco dimethyl sulfopropyl betaine, stearyl dimethyl sulfopropyl betaine, lauryl dimethyl sulfoethyl betaine, lauryl bis(2-hydroxyethyl) sulfopropyl betaine), alkyl amido betaines, 4-[N,N-di(2-hydroxyethyl)-N-octadecylammonio]-butane-1-carboxylate; 5-[S-3-hydroxypropyl-5-hexadecylsulfonio]-3-hydroxy-pentanel-sulfate; 3-[P,P-diethyl-P-3,6,9-trioxatetradexoxylphosphonio]-2-hydroxy-propane-1-phosphate; 3-[N,N-dipropyl-N-3-dodecoxy-2-hydroxypropylammonio]-propane-1-phosphate; 3-(N,N-dimethyl-N-hexadecylammonio)propane-1-sulfonate; 3-(N,N-dimethyl-N-hexadecylammonio)-2-hydroxy-propane-1-sulfonate; 4-[N,N-di-(2-hydroxy-ethyl)-N-(2-hydroxydodecyl)ammonio]-butane-1-carboxylate; 3-[S-ethyl-S-(3-dodecoxy-2-hydroxypropyl)sulfonio]-propane-1-phosphate; 3-[P,P-dimethyl-P-dodecylphosphonio]-propane-1-phosphonate; and 5-[N,N-di(3-hydroxypropyl)-N-hexadecylammonio]-2-hydroxypentane-1-sulfate, sodium 3-dodecylaminopropane sulfonate; alkyl amphosulfonates; alkyl amphosulfosuccinates; oleoamphopropylsulfonate, and cocoamphopropylsulfonate; polyethylene oxide condensates; long chain tertiary phosphine oxides; long chain dialkyl sulfoxides; Silicone copolyols (e.g., dimethicone copolyols), stearamide diethanolamide (DEA), cocamide monoethanolamide (MEA), glyceryl monoleate, sucrose stearate, Cetheth-2, Poloxamer 181, hydrogenated tallow amide DEA, polyoxyethylene 4 sorbitol beeswax derivative (ATLAS 6-1702), polyoxyethylene 2 cetyl ether (BRIJ 52), polyoxyethylene 2 stearyl ether (BRIJ 72), polyoxyethylene 2 oleyl ether (BRIJ 92), polyoxyethylene 2 oleyl ether (BRIJ 93), sorbitan monopalmitate (SPAN 40), sorbitan monostearate (SPAN 60), sorbitan tristearate (SPAN 65), sorbitan monoleate, NF (SPAN 80) sorbitan trioleate (SPAN 85), fluorinated alkyl quaternary ammonium iodide; mixed mono- and bis-perfluoroalkyl phosphates, ammonium salts; mixed mono- and bis-fluoroalkyl phosphate, ammonium salts, complexed with aliphatic quaternary methosulfates; perfluoroalkyl sulfonic acid, ammonium salts; mixed telomer phosphate diethanolamine salts; amine perfluoroalkyl sulfonates; ammonium perfluoroalkyl sulfonates; potassium perfluoroalkyl sulfonates; potassium fluorinated alkyl carboxylates; ammonium perfluoroalkyl sulfonates; and ammonium perfluoroalkyl carboxylates; sodium dioctyl sulfosuccinate; magnesium dioctyl sulfosuccinate; ammonium dioctyl sulfosuccinate; sodium dodecyl sulfate; magnesium dodecyl sulfate; ammonium dodecyl sulfate; cocoamidopropyl betaine sodium laureth sulfate; sodium dinonyl sulfo succinate; sodium decyl sulfate; sodium alpha olefin sulfonate; sodium laureth sulfate; magnesium laureth sulfate; ammonium laureth sulfate; cocoamidopropyl betaine; polyethoxylated glycol ether of glyceryl isostewarate; polyethoxylated glycol ether of glyceryl monooleate; PEG-30 glyceryl isostearate; polyoxyethylene glycerol monoleate; polyethylene glycol; PPG-18; PPG-10; 18 dimethicone; 1 dimethicon; cetyl polyethylene glycol; glyceryl monostearate; laureth-23; and PEG 75 lanolin. In certain embodiments, the surfactant, emulsifier, or detergent is a silicon-containing chemical compound. Exemplary silicon-based detergents, emulsifiers, or surfactants useful in cosmetic compositions include dimethicone, cyclopentasiloxane, cyclohexasiloxane, PEG/dimethicone copolymers, PPG/dimethicone copolymers, phenyltrimethicone, alkyl silicones, amodimethicone, silicone quaternium-18, and dimethiconol.

Many surfactants useful in the inventive compositions are described in McCutcheon's Detergents and Emulsifiers, 1984 Annual, published by Allured Publishing Corporation, which is incorporated herein by reference. Other useful surfactant in the inventive compositions include those described in U.S. Pat. Nos. 2,396,278; 2,438,091; 2,486,921; 2,486,922; 2,528,378; 2,658,072; 3,155,591; 3,332,880; 3,929,678; 3,959,461; 3,993,744; 3,993,745; 4,122,029; 4,176,176; 4,265,878; 4,275,055; 4,387,090; and 4,421,769; each of which is incorporated herein by reference. The inventive compositions contain a surfactant in the range of from about 0.01% to about 20% by weight. In certain embodiments, the composition contains a surfactant in the range from about 0.1% to about 10% by weight. In certain embodiments, the composition contains surfactant in the range from about 0.5% to about 15% by weight. In certain embodiments, the composition contains surfactant in the range from about 2% to about 10% by weight.

### Solvent

The inventive cosmetic compositions typically include a solvent or combination of solvents to dissolve or solubilize the components of the composition. The solvent typically makes up the balance of a composition. Exemplary solvents useful in the inventive compositions include Acetone, Alcohol, Alcohol Denat., Amyl Acetate, Benzyl Alcohol, Benzyl Benzoate, Benzyl Glycol, Benzyl Laurate, Benzyl Laurate/Myristate/Palmitate, 1,4-Butanediol, 2,3-Butanediol, Buteth-3, Butoxydiglycol, Butoxyethanol, Butoxyethyl Acetate, Butyl Acetate, n-Butyl Alcohol, t-Butyl Alcohol, Butylene Glycol, Butylene Glycol Propionate, Butyl Ethylpropanediyl Ethylhexanoate, Butyl Lactate, Butyloctanol, Butyloctyl Benzoate, Butyloctyl Salicylate, Butylphthalimide, Butyrolactone, C8-12 Acid Triglyceride, C12-18 Acid Triglyceride, C9-12 Alkane, C10-13 Alkane, C13-14 Alkane, C13-15 Alkane, C14-17 Alkane, C14-19 Alkane, C15-19 Alkane, C15-23 Alkane, C18-21 Alkane, C8-9 Alkane/Cycloalkane, C9-10 Alkane/Cycloalkane, C9-11 Alkane/Cycloalkane, C9-16 Alkane/Cycloalkane, C10-12 Alkane/Cycloalkane, C11-14 Alkane/Cycloalkane, C11-15 Alkane/Cycloalkane, C12-13 Alkane/Cycloalkane, C8-10 Alkane/Cycloalkane/Aromatic Hydrocarbons, C12-15 Alkane/Cycloalkane/Aromatic Hydrocarbons, C9-10 Aromatic Hydrocarbons, C10-11 Aromatic Hydrocarbons, CD Alcohol 19, Chlorinated Paraffin, C7-8 Isoparaffin, C8-9 Isoparaffin, C9-11 Isoparaffin, C9-13 Isoparaffin, C9-14 Isoparaffin, C9-16 Isoparaffin, C10-11 Isoparaffin, C10-12 Isoparaffin, C10-13 Isoparaffin, C22-12 Isoparaffin, C11-13 Isoparaffin, C11-14 Isoparaffin, C12-14 Isoparaffin, C12-20 Isoparaffin, C13-14 Isoparaffin, C13-16 Isoparaffin, C20-40 Isoparaffin, Coix Lacryma-Jobi (Job's Tears) Seed Water, C6-12 Perfluoroalkylethanol, Crotamiton, C10-18 Triglycerides, Cycloethoxymethicone, Cycloheptasiloxane, Cyclohexane, Cyclohexanedimethanol, Cyclohexasiloxane, Cyclomethicone, Cyclopentasiloxane, Cyclotetrasiloxane, Cyclotrisiloxane, Decane, 1,10-Decanediol, Decene, Deodorized Kerosene, Diacetin, Diacetone Alcohol, Dibutyl Adipate, Dibutyloctyl Malate, Dibutyloctyl Sebacate, Dibutyl Oxalate, Dibutyl Phthalate, Dibutyl Sebacate, Di-C12-15 Alkyl Maleate, Diethoxydiglycol, Diethoxyethyl Succinate, Diethylene Glycol, Diethylhexyl Adipate, Diethylhexyl 2,6-Naphthalate, Diethylhexyl Phthalate, Diethylhexyl Sebacate, Diethylhexyl Succinate, Diethyl Oxalate, Diethyl Phthalate, Diethyl Sebacate, Diethyl Succinate, Diheptylundecyl Adipate, Dihexyl Adipate, Dihexyldecyl Sebacate, Diisoamyl Malate, Diisobutyl Adipate, Diisobutyl Oxalate, Diisocetyl Adipate, Diisodecyl Adipate, Diisononyl Adipate, Diisooetyl Adipate, Diisopropyl Adipate, Diisopropyl Oxalate, Diisopropyl Sebacate, Dimethoxydiglycol, Dimethyl Adipate, Dimethyl Capramide, Dimethyl Carbonate, Dimethyl Ether, Dimethyl Glutarate, Dimethyl Isosorbide, Dimethyl Maleate, Dimethyl Oxalate, Dimethyl Phthalate, Dimethyl Succinate, Dimethyl Sulfone, Dioctyldodecyl Sebacate, Dioxolane, Diphenyl Methane, Di-PPG-3 Myristyl Ether Adipate, Dipropyl Adipate, Dipropylene Glycol, Dipropylene Glycol Dimethyl Ether, Dipropyl Oxalate, Ditridecyl Adipate, Dodecene, Echium Plantagineum Seed Oil, Eicosane, Ethoxydiglycol, Ethoxydiglycol Acetate, Ethoxyethanol, Ethoxyethanol Acetate, Ethyl Acetate, Ethylene Carbonate, Ethyl Ether, Ethyl Hexanediol, Ethylhexyl Benzoate, Ethyl Lactate, Ethyl Perfluorobutyl Ether, Furfural, Glycereth-7 Benzoate, Glycereth-18 Benzoate, Glycereth-20 Benzoate, Glycereth-7 Diisononanoate, Glycereth-5 Lactate, Glycereth-7 Lactate, Glycereth-7 Triacetate, Glycofurol, Glycol, Heptane, Hexadecene, Hexane, Hexanediol, 1,2-Hexanediol, 1,2,6-Hexanetriol, Hexene, Hexyl Alcohol, Hexyldecyl Benzoate, Hexyldodecyl Salicylate, Hexylene Glycol, Hydrogenated Polydecene, Hydrogenated Polydodecene, Hydroxymethyl Dioxolanone, Isoamyl Acetate, Isobutoxypropanol, Isobutyl Acetate, Isobutyl Benzoate, Isododecane, Isoeicosane, Isohexadecane, Isooctane, Isopentane, Isopentyldiol, Isopropyl Acetate, Isopropyl Alcohol, Isopropyl Citrate, Isopropyl phthal imide, Isostearyl Glycolate, Limonene, MEK, 3-Methoxybutanol, Methoxydiglycol, Methoxyethanol, Methoxyethanol Acetate, Methoxyisopropanol, Methoxyisopropyl Acetate, Methoxymethylbutanol, Methoxy PEG-7, Methoxy PEG-10, Methoxy PEG-16, Methoxy PEG-25, Methoxy PEG-40, Methoxy PEG-100, Methyl Acetate, Methylal, Methyl Alcohol, Methyl Benzoate, Methylbutenes, Methyl Gluceth-20 Benzoate, Methyl Hexyl Ether, Methyl Lactate, Methyl Perfluorobutyl Ether, Methylpropanediol, Methyl Pyrrolidone, Methyl Sunflowerseedate, Methyl Trimethicone, MIBK, Mineral Oil, Mineral Spirits, Mixed Terpenes, Momordica Grosvenori Fruit Juice, Mustelic/Palmitic Triglyceride, Neopentyl Glycol, Octadecane, Octadecene, Octane, Octene, Oleyl Alcohol, PEG-4, PEG-6, PEG-7, PEG-8, PEG-9, PEG-10, PEG-12, PEG-14, PEG-16, PEG-18, PEG-20, PEG-32, PEG-33, PEG-40, PEG-45, PEG-55, PEG-60, PEG-75, PEG-80, PEG-90, PEG-100, PEG-135, PEG-150, PEG-180, PEG-200, PEG-220, PEG-240, PEG-350, PEG-400, PEG-450, PEG-500, PEG-2 Benzyl Ether, PEG-15 Butanediol, PEG-3 Methyl Ether, PEG-4 Methyl Ether, PEG-6 Methyl Ether, PEG-7 Methyl Ether, PEG/PPG-1/2 Copolymer, PEG/PPG-4/2 Copolymer, PEG/PPG-5/30 Copolymer, PEG/PPG-6/2 Copolymer, PEG/PPG-7/50 Copolymer, PEG/PPG-8/17 Copolymer, PEG/PPG-10/70 Copolymer, PEG/PPG-17/6 Copolymer, PEG/PPG-18/4 Copolymer, PEG/PPG-19/21 Copolymer, PEG/PPG-23/17 Copolymer, PEG/PPG-23/50 Copolymer, PEG/PPG-25/30 Copolymer, PEG/PPG-26/31 Copolymer, PEG/PPG-30/33 Copolymer, PEG/PPG-35/9 Copolymer, PEG/PPG-38/8 Copolymer, PEG/PPG-116/66 Copolymer, PEG/PPG-125/30 Copolymer, PEG/PPG-160/31 Copolymer, PEG/PPG-200/70 Copolymer, PEG/PPG-240/60 Copolymer, PEG-10 Propylene Glycol, Pentane, 1,5-Pentanediol, Pentylene Glycol, Perfluorocaprylyl Bromide, Perfluorodecalin, Perfluorodimethylcyclohexane, Perfluorohexane, Perfluoromethylcyclopentane, Perfluoroperhydrobenzyl Tetralin, Perfluoroperhydrophenanthrene, Perfluorotetralin, Petroleum Distillates, Phenoxyisopropanol, Phenylpropanol, Polyperfluoroethoxymethoxy Difluoromethyl Ether, PPG-3, PPG-7, PPG-10 Butanediol, PPG-2-Buteth-3, PPG-3-Buteth-5, PPG-5-Buteth-7, PPG-7-Buteth-4, PPG-7-Buteth-10, PPG-12-Buteth-16, PPG-15-Buteth-20, PPG-20-Buteth-30, PPG-2 Butyl Ether, PPG-3 Butyl Ether, PPG-24-Glycereth-24, PPG-25-Glycereth-22, PPG-10 Glyceryl Ether, PPG-55 Glyceryl Ether, PPG-67 Glyceryl Ether, PPG-70 Glyceryl Ether, PPG-2 Methyl Ether, PPG-3 Methyl Ether, PPG-2 Methyl Ether Acetate, PPG-2 Propyl Ether, Propanediol, Propyl Acetate, Propyl Alcohol, Propylene Carbonate, Propylene Glycol, Propylene Glycol Butyl Ether, Propylene Glycol Propyl Ether, SD Alcohol 1, SD Alcohol 3-A, SD Alcohol 3-B, SD Alcohol 3-C, SD Alcohol 23-A, SD Alcohol 23-F, SD Alcohol 23-H, SD Alcohol 27-B, SD Alcohol 30, SD Alcohol 31-A, SD Alcohol 36, SD Alcohol 37, SD Alcohol 38-B, SD Alcohol 38-C, SD Alcohol 38-D, SD Alcohol 38-F, SD Alcohol 39, SD Alcohol 39-A, SD Alcohol 39-B, SD Alcohol 39-C, SD Alcohol 39-D, SD Alcohol 40, SD Alcohol 40-A, SD Alcohol 40-B, SD Alcohol 40-C, SD Alcohol 46, Sea Water, Shark Liver Oil, Sorbeth-6, Sorbeth-20, Sorbeth-30, Sorbeth-40, Stearyl Benzoate, Tetradecene, Tetradecylpropionates, Tetrahydrofurfuryl Acetate, Tetrahydrofurfuryl Alcohol, Thiolanediol, Toluene, Triacetin, Tributyl Citrate, Tributylcresylbutane, Trichloroethane, Triethyl Phosphate, Trimethylhexanol, Trimethyl Pentanol Hydroxyethyl Ether, Water, and xylene. In certain embodiments, the solvent is acetic acid, acetone, alcohol, alcohol (denatured), benzophenone, butoxydiglycol, butyl acetate, n-butyl acetate, n-butyl alcohol, butylene glycol, butyl myristate, butyloctyl benzoate, butyloctyl salicylate, butyl stearate, C12-15 alkyl benzoate, capric acid, caprylic alcohol, cetearyl octanoate, cetyl stearyl octanoate, chlorobutanol, C9-11 isoparaffin, C10-11 isoparaffin, C10-13 isoparaffin, decyl alcohol, diethylene glycol, diethylene glycol dibenzoate, diethylhexyl maleate, diethylhexyl 2,6-naphthalate, diethyl sebacate, diisocetyl adipate, diisopropyl adipate, diisopropyl sebacate, dimethylphthalate, dioctyl adipate, dioctyl succinate, dipropylene glycol, dipropylene glycol dibenzoate, ethoxydiglycol, ethyl acetate, ethyl lactate, ethyl macadamiate, ethyl myristate, ethyl oleate, glycereth-7 benzoate, glycereth-7 diisononanoate, glycereth-4,5-lactate, glycereth-7 triacetate, glycerin, glycine soja (soybean) oil, glycofurol, heptane, hexyl alcohol, hexyldecyl benzoate, hexylene glycol, isobutyl stearate, isocetyl salicylate, isodecyl benzoate, isodecyl isononanoate, isodecyl octanoate, isodecyl oleate, isododecane, isoeicosane, isohexadecane, isononyl isononanoate, isooctane, isopropyl alcohol, isopropyl laurate, isopropyl myristate, isopropyl palmitate, isostearyl stearoyl stearate, laneth-5, lanolin oil, laureth-2 acetate, MEK, methoxydiglycol, methyl acetate, methyl alcohol, methylene chloride, methylpropanediol, methylsoyate, MIBK, morpholine, neopentyl glyol, neopentyl glyol dioctanoate, nonocynol-9, octyl benzoate, octyldodecyl lactate, octyldodecyl octyldodecanoate, octyl isononanoate, octyl isostearate, octyl laurate, octyl palmitate, octyl stearate, oleyl alcohol, olive oil PEG-6 esters, peanut oil PEG-6 esters, PEG-12, PBG-33 castor oil, PEG-50 glyceryl cocoate, PEG-20 hydrogenated castor oil, PEG-6 methyl ether, penetaerythrity tetracaprylate/tetracaprate, pentane, petroleum distillates, polyglyceryl-3 diisostearate, polyglyceryl-2 dioleate, polyoxyethylene glycol dibenzoate, PPG-3, PPG-20 lanolin alcohol ether, PPG-2 myristyl ether propionate, propyl alcohol, propylene carbonate, propylene glycol, propylene glycol caprylate, propylene glycol dibenzoate, propylene glycol methyl ether, propylene glycol myristate, pyridine, ricinus communis (castor) seed oil, sesamum indicum (sesame) oil, sorbitan trioleate, stearyl heptaroate, toluene, 2,2,4-timethylpentane, and xylene. In a certain embodiment, the solvent is selected from the group consisting of polyethylene glycol, propylene glycol, ethanol, isopropanol, n-butanol, water, and mixtures thereof. In certain embodiments, the solvent comprises a mixture of propylene glycol and denatured ethanol. In certain embodiments, the composition includes water as a solvent. Nonaqueous solvents may also be used in the inventive cosmetic compositions. In certain embodiments, the solvent is volatile. The solvent may make up from about 1% to about 99% by weight of the composition. In certain embodiments, the solvent is from about 5% to about 99% by weight of the composition. In certain embodiments, the solvent is from about 50% to about 99% by weight of the composition. In certain embodiments, the solvent is from about 80% to about 99% by weight of the composition.

### Salts

Various salts may also be added to the inventive compositions. Salts are typically ionized and result in stoichiometrically equivalent amounts of cations and anions when dissolved in a solution (e.g., water). Salts are typically soluble in water. The salt used in the inventive compositions may be an inorganic salt or an organic salt. Salts are typically used in compositions as thickening agents, buffering agents, hair waving agents, humectants, and/or oxidizing or reducing agents. In certain embodiments, the salt used in the composition is an inorganic salt resulting from the reaction of an inorganic base with an inorganic acid. Under such circumstances, the base provides the cation while the acid provides the anion. Exemplary inorganic salts useful in the present invention include Aluminum Bromohydrate, Aluminum Chloride, Aluminum Chlorohydrate, Aluminum Chlorohydrex, Aluminum Chlorohydrex PEG, Aluminum Chlorohydrex PG, Aluminum Dichlorohydrate, Aluminum Dichlorohydrex PEG, Aluminum Dichlorohydrex PG, Aluminum Fluoride, Aluminum Sesquichlorohydrate, Aluminum Sesquichlorohydrex PEG, Aluminum Sesquichlorohydrex PG, Aluminum Silicate, Aluminum Sulfate, Aluminum Zirconium Octachlorohydrate, Aluminum Zirconium Octachlorohydrex GLY, Aluminum Zirconium Pentachlorohydrate, Aluminum Zirconium Pentachlorohydrex GLY, Aluminum Zirconium Tetrachlorohydrate, Aluminum Zirconium Tetrachlorohydrex GLY, Aluminum Zirconium Tetrachlorohydrex PEG, Aluminum Zirconium Tetrachlorohydrex PG, Aluminum Zirconium Trichlorohydrate, Aluminum Zirconium Trichlorohydrex GLY, Ammonium Alum, Ammonium Bicarbonate, Ammonium Bisulfite, Ammonium Carbamate, Ammonium Carbonate, Ammonium Chloride, Ammonium Fluoride, Ammonium Fluorosilicate, Ammonium Hexafluorophosphate, Ammonium Iodide, Ammonium Monofluorophosphate, Ammonium Nitrate, Ammonium Persulfate, Ammonium Phosphate, Ammonium Sulfate, Ammonium Sulfite, Ammonium Thiocyanate, Barium Chloride, Barium Sulfate, Barium Sulfide, Bismuth Oxychloride, Bismuth Subnitrate, Bittern, Boron Nitride, Calcium Carbonate, Calcium Chloride, Calcium Dihydrogen Phosphate, Calcium Fluoride, Calcium Monofluorophosphate, Calcium Phosphate, Calcium Pyrophosphate, Calcium Silicate, Calcium Sulfate, Calcium Sulfide, Calcium Titanate, Cobalt Chloride, Copper Sulfate, Cupric Chloride, Diammonium Phosphate, Dipotassium Phosphate, Disodium Phosphate, Disodium Pyrophosphate, Ferric Ammonium Ferrocyanide, Ferric Chloride, Ferric Ferrocyanide, Ferrous Sulfate, Hydroxylamine HCI, Hydroxylamine Sulfate, Lanthanum Chloride, Lithium Fluoride, Lithium Magnesium Silicate, Lithium Sulfide, Magnesium Aluminum Silicate, Magnesium Bromide, Magnesium Carbonate, Magnesium Carbonate Hydroxide, Magnesium Chloride, Magnesium Fluoride, Magnesium Fluorosilicate, Magnesium Hydrogen Phosphate, Magnesium Phosphate, Magnesium Silicate, Magnesium Sulfate, Magnesium Sulfide, Magnesium Trisilicate, Manganese Chloride, Manganese Sulfate, Manganese Violet, Mineral Salts, Pentapotassium Triphosphate, Pentasodium Triphosphate, Potassium Alum, Potassium Bicarbonate, Potassium Borate, Potassium Bromate, Potassium Bromide, Potassium Carbonate, Potassium Caroate, Potassium Chlorate, Potassium Chloride, Potassium Fluoride, Potassium Fluorosilicate, Potassium Iodide, Potassium Metabisulfite, Potassium Monofluorophosphate, Potassium Nitrate, Potassium Persulfate, Potassium Phosphate, Potassium Polyphosphate, Potassium Silicate, Potassium Sulfate, Potassium Sulfide, Potassium Sulfite, Potassium Tetrathionate, Potassium Thiocyanate, Sea Salt, Selenium Sulfide, Silver Chloride, Silver Nitrate, Silver Sulfate, Sodium Alum, Sodium Aluminate, Sodium Bicarbonate, Sodium Bisulfate, Sodium Bisulfite, Sodium Borate, Sodium Bromate, Sodium Carbonate, Sodium Carbonate Peroxide, Sodium Chlorate, Sodium Chloride, Sodium Fluoride, Sodium Fluorosilicate, Sodium Hexametaphosphate, Sodium Hydrosulfite, Sodium Iodate, Sodium Iodide, Sodium Magnesium Silicate, Sodium Metabisulfite, Sodium Metaphosphate, Sodium Metasilicate, Sodium Molybdate, Sodium Monofluorophosphate, Sodium Nitrate, Sodium Nitrite, Sodium Perborate, Sodium Persulfate, Sodium Phosphate, Sodium Polyphosphate, Sodium Sesquicarbonate, Sodium Silicate, Sodium Silicoaluminate, Sodium Stannate, Sodium Sulfate, Sodium Sulfide, Sodium Sulfite, Sodium Thiocyanate, Sodium Thiosulfate, Sodium Trimetaphosphate, Stannous Chloride, Stannous Fluoride, Stannous Pyrophosphate, Strontium Chloride, Strontium Chloride Hexahydrate, Strontium Sulfide, Tetrapotassium Pyrophosphate, Tetrasodium Pyrophosphate, Tricalcium Phosphate, Trimagnesium Phosphate, Trisodium Phosphate, Zinc Borate, Zinc Carbonate, Zinc Carbonate Hydroxide, Zinc Chloride, Zinc Sulfate, Zinc Sulfide, Zirconium Chlorohydrate, Zirconium Silicate, and Zirconyl Chloride. In certain embodiments, the salt used in the composition is an organic salt. In certain embodiments, organic salts result from reacting an organic base with an inorganic or organic acid or from reacting an inorganic base with an organic acid. In certain embodiments, the organic salt is a salt of a carboxylic acid. In certain embodiments, the organic salt is a salt of a fatty acid. In certain embodiments, the organic salt is a salt of an amine. In certain particular embodiments, the salt is a salt of a quaternary ammonium compound. Exemplary organic salts useful in accordance with the present invention include Alanine Glutamate, Allantoin Acetyl Methionine, Allantoin Ascorbate, Allantoin Biotin, Allantoin Calcium Pantothenate, Allantoin Galacturonic Acid, Allantoin Glycyrrhetinic Acid, Allantoin PABA, Allantoin Polygalacturonic Acid, Aluminum Acetate, Aluminum Acetate Solution, Aluminum Benzoate, Aluminum Butoxide, Aluminum Citrate, Aluminum Diacetate, Aluminum Dicetyl Phosphate, Aluminum Lactate, Aluminum/Magnesium Hydroxide Stearate, Aluminum Methionate, Aluminum PCA, Aluminum Sucrose Octasulfate, Aluminum Triformate, Ammonium Acetate, Ammonium Benzoate, Ammonium Caseinate, Ammonium C6-16 Perfluoroalkylethyl, Phosphate, Ammonium Glycolate, Ammonium Glycyrrhizate, Ammonium Lactate, Ammonium Laureth-6 Carboxylate, Ammonium Laureth-8 Carboxylate, Ammonium Phosphatidyl Rapeseedate, Ammonium Propionate, Ammonium Shellacate, Ammonium Thioglycolate, Ammonium Thiolactate, Amodimethicone Hydroxystearate, Antimony Potassium Tartrate, Arginine Aspartate, Arginine Bicarbonate, Arginine DNA, Arginine Ferulate, Arginine Glutamate, Arginine Hexyldecyl Phosphate, Arginine PCA, Barium Gluconate, Behentrimonium Dimethicone PEG-8 Phthalate, Bismuth Citrate, Bismuth Subgallate, Brucine Sulfate, Calcium Acetate, Calcium Ascorbate, Calcium Aspartate, Calcium Benzoate, Calcium Carboxymethyl Cellulose, Calcium Citrate, Calcium Cyclamate, Calcium Disodium EDTA, Calcium DNA, Calcium Fructoborate, Calcium Fructoheptonate, Calcium Glucoheptonate, Calcium Gluconate, Calcium Glycerophosphate, Calcium Lactate, Calciuim Pantetheine Sulfonate, Calcium Pantothenate, Calcium Paraben, Calcium PCA, Calcium Potassium Carbomer, Calcium Propionate, Calcium Saccharin, Calcium Salicylate, Calcium Sorbate, Calcium Starch Isododecenylsuccinate, Calcium Stearoyl Lactylate, Calcium Tartrate, Calcium Thioglycolate, Cellulose Gum, Chitosan Adipate, Chitosan Ascorbate, Chitosan Glycolate, Chitosan Salicylate, Chloramine T, Chlorhexidine Diacetate, Chlorhexidine Digluconate, Chlorhexidine Dihydrochloride, Chlorophyllin-Copper Complex, Chlorophyllin-Iron Complex, Ciclopirox Olamine, Cobalt Gluconate, Copper DNA, Copper Gluconate, Copper PCA, Copper PCA Methylsilanol, Copper Picolinate, Copper Tripeptide-1, Copper Usnate, Cupric Acetate, Cysteamine HCI, Cysteine DNA, DEA-Cetyl Phosphate, DEA-Hydrolyzed Lecithin, DEA-Methoxycinnamate, Diammonium Citrate, Diammonium Dithiodiglycolate, Dibehenamidopropyldimethylamine Dilinoleate, Dibromopropamidine Diisethionate, Diglycol Guanidine Succinate, Dihydroxyethyl Tallowamine Oleate, Dilithium Oxalate, Dimethicone Propylethylenediamine Behenate, Dipotassium Azelate, Dipotassium Glycyrrhizate, Dipotassium Oxalate, Disodium Adenosine Phosphate, Disodium Adenosine Triphosphate, Disodium Ascorbyl Sulfate, Disodium Azelate, Disodium Cocaminopropyl Iminodiacetate, Disodium Coco-Glucoside Citrate, Disodium Coco-Glucoside Sulfosuccinate, Disodium Cocoyl Glutamate, Disodium Cupric Citrate, Disodium Cystinyl Disuccinate, Disodium Dicarboxyethyl Cocopropylenediamine, Disodium Fumarate, Disodium Glycyrrhizate, Disodium PPG-2-Isodeceth-7 Carboxyampho-, diacetate, Disodium Rutinyl Disulfate, Disodium Sebacate, Disodium Succinate, Disodium Succinoyl Glycyrrhetinate, Disodium Tartrate, Disodium Tetrapropenyl Succinate, Disoyamidoethyl Hydroxyethyl Ammonium, Lactate, Ethanolamine Dithiodiglycolate, Ethanolamine Glycerophosphate, Ethanolamine Thioglycolate, Ethyl Hydroxy Picolinium Lactate, Ethyl Lauroyl Arginate HCI, Ferric Ammonium Citrate, Ferric Citrate, Ferric Glycerophosphate, Ferrous Aspartate, Ferrous Glucoheptonate, Ferrous Gluconate, Guanidine Carbonate, Guanidine HCI, Guanidine Phosphate, Hexamidine Diisethionate, Hexamidine Paraben, Iron Picolinate, Isopropyl Titanium Triisostearate, Isostearamidopropyl Dimethylamine Gluconate, Isostearamidopropyl Dimethylamine Glycolate, Isostearamidopropyl Dimethylamine Lactate, Isostearamidopropyl Morpholine Lactate, Lauryl Isoquinolinium Saccharinate, Lauryl PCA, Lead Acetate, Lithium Gluconate, Lithium Oxidized Polyethylene, Lysine DNA, Lysine Glutamate, Magnesium Acetate, Magnesium Ascorbate, Magnesium Ascorbate/PCA, Magnesium Ascorbyl Phosphate, Magnesium Benzoate, Magnesium Citrate, Magnesium DNA, Magnesium Glucoheptonate, Magnesium Gluconate, Magnesium Glycerophosphate, Magnesium Laureth-11 Carboxylate, Magnesium PCA, Magnesium Propionate, Magnesium Salicylate, Magnesium Thioglycolate, Manganese Gluconate, Manganese Glycerophosphate, Manganese PCA, MEA-Benzoate, MEA Biotinate, MEA-Cocoate, MEA-Dicetearyl Phosphate, MEA-Laureth-6 Carboxylate, MEA o-Phenylphenate, MEA PPG-6 Laureth-7 Carboxylate, MEA-PPG-8-Steareth-7 Carboxylate, MEA-Salicylate, MEA-Thiolactate, MEA-Undecylenate, Methyl Hydroxycetyl Glucaminium Lactate, Methylsilanol Hydroxyproline Aspartate, Molybdenum Aspartate, Monosodium Citrate, Nickel Gluconate, Nicotinyl Tartrate, Olivamidopropyl Dimethylamine Lactate, Oxyquinoline Benzoate, Oxyquinoline Sulfate, PCA Ethyl Cocoyl Arginate, Pentasodium Ethylenediamine Tetramethylene Phosphonate, Pentasodium Pentetate, Phenyl Mercuric Acetate, Phenyl Mercuric Benzoate, Phenyl Mercuric Borate, Phenyl Mercuric Bromide, Phenyl Mercuric Chloride, Phytosphingosine Glycolate, Phytosphingosine HCI, Phytosphingosine Lactate, Piroctone Olamine, Potassium Acesulfame, Potassium Acetate, Potassium Ascorbylborate, Potassium Ascorbyl Tocopheryl Phosphate, Potassium Azeloyl Diglycinate, Potassium Benzoate, Potassium Biphthalate, Potassium Butylparaben, Potassium C12-13 Alkyl Phosphate, Potassium Carbomer, Potassium Cellulose Succinate, Potassium Citrate, Potassium Cocoyl Glutamate, Potassium Cocoyl Glycinate, Potassium Cocoyl PCA, Potassium Cyanate, Potassium DNA, Potassium Ethylparaben, Potassium Fructoborate, Potassium Glucoheptonate, Potassium Gluconate, Potassium Glycerophosphate, Potassium Glycol Sulfate, Potassium Glycyrrhetinate, Potassium Glycyrrhizinate, Potassium Hydroxycitrate, Potassium Lactate, Potassium Lanolate, Potassium Laureth-3 Carboxylate, Potassium Laureth-4 Carboxylate, Potassium Laureth-5 Carboxylate, Potassium Laureth-6 Carboxylate, Potassium Laureth-10 Carboxylate, Potassium Lauroyl Methyl Beta-Alanine, Potassium Lauroyl PCA, Potassium Lauryl Phosphate, Potassium Methoxycinnamate, Potassium Methylparaben, Potassium Oxidized Microcrystalline Wax, Potassium Paraben, Potassium PCA, Potassium PEG-50 Hydrogenated Castor Oil Succinate, Potassium Phenoxide, Potassium Phenylbenzimidazole Sulfonate, Potassium o-Phenylphenate, Potassium Propionate, Potassium Propylparaben, Potassium Salicylate, Potassium Sodium Tartrate, Potassium Sorbate, Potassium Tartrate, Potassium Taurate, Potassium Taurine Laurate, Potassium Thioglycolate, Potassium Trideceth-3 Carboxylate, Potassium Trideceth-4 Carboxylate, Potassium Trideceth-7 Carboxylate, Potassium Trideceth-15 Carboxylate, Potassium Trideceth-19 Carboxylate, Potassium Troclosene, Potassium Undecylenoyl Alginate, Pyridoxine HCI, Saccharated Lime, Sodium Acetate, Sodium Allantoin PCA, Sodium Aluminum Chlorohydroxy Lactate, Sodium Aluminum Lactate, Sodium Ascorbate, Sodium Ascorbyl Phosphate, Sodium Behenoyl Lactylate, Sodium Benzoate, Sodium Butoxyethoxy Acetate, Sodium Butylparaben, Sodium Caproyl Lactylate, Sodium Capryleth-2 Carboxylate, Sodium Capryleth-9 Carboxylate, Sodium Carbomer, Sodium Carboxydecyl PEG-8 Dimethicone, Sodium Carboxyethyl Tallow Polypropylamine, Sodium Carboxymethyl Cocopolypropylamine, Sodium Carboxymethyl Lauryl Glucoside, Sodium Carboxymethyl Oleyl Polypropylamine, Sodium Carboxymethyl Starch, Sodium Carboxymethyl Tallow Polypropylamine, Sodium Ceteth-13 Carboxylate, Sodium p-Chloro-m-Cresol, Sodium Citrate, Sodium Coco-Glucoside Tartrate, Sodium Cocoyl Glycinate, Sodium Cocoyl Lactylate, Sodium Cocoyl Methyl Beta-Alanine, Sodium C9-11 Pareth-6 Carboxylate, Sodium C11-15 Pareth-7 Carboxylate, Sodium C12-13 Pareth-5 Carboxylate, Sodium C12-13 Pareth-8 Carboxylate, Sodium C12-13 Pareth-12 Carboxylate, Sodium C12-15 Pareth-6 Carboxylate, Sodium C12-15 Pareth-7 Carboxylate, Sodium C12-15 Pareth-8 Carboxylate, Sodium C14-15 Pareth-8 Carboxylate, Sodium C12-14 Sec-Pareth-8 Carboxylate, Sodium Cyclamate, Sodium Cyclodextrin Sulfate, Sodium Cyclopentane Carboxylate, Sodium Deceth-2 Carboxylate, Sodium Dehydroacetate, Sodium Dilaureth-7 Citrate, Sodium Erythorbate, Sodium Ethylparaben, Sodium Formate, Sodium Fructoborate, Sodium Fumarate, Sodium Gluceptate, Sodium Gluconate, Sodium Glucuronate, Sodium Glycerophosphate, Sodium Hexeth-4 Carboxylate, Sodium Hinokitiol, Sodium Hydrolyzed Casein, Sodium Hydroxymethane Sulfonate, Sodium Isethionate, Sodium Isobutylparaben, Sodium Isoferulate, Sodium Isopropylparaben, Sodium Isosteareth-6 Carboxylate, Sodium Isosteareth-11 Carboxylate, Sodium Isostearoyl Lactate, Sodium Isostearoyl Lactylate, Sodium Lactate, Sodium Lactate Methylsilanol, Sodium Laureth-3 Carboxylate, Sodium Laureth-4 Carboxylate, Sodium Laureth-5 Carboxylate, Sodium Laureth-6 Carboxylate, Sodium Laureth-8 Carboxylate, Sodium Laureth-11 Carboxylate, Sodium Laureth-12 Carboxylate, Sodium Laureth-13 Carboxylate, Sodium Laureth-14 Carboxylate, Sodium Laureth-16 Carboxylate, Sodium Laureth-17 Carboxylate, Sodium Laureth-7 Tartrate, Sodium Lauroyl Aspartate, Sodium Lauroyl Lactylate, Sodium Lauroyl/Myristoyl Aspartate, Sodium Lauryl Glucosideoxyacetate, Sodium Lauryl Glycol Carboxylate, Sodium Levulinate, Sodium Malate, Sodium Mannuronate Methylsilanol, Sodium Methoxy PPG-2 Acetate, Sodium Methylesculetin Acetate, Sodium Methylparaben, Sodium Myristoyl Methyl Beta-Alanine, Sodium Naphthol Sulfonate, Sodium 5-Nitroguaiacolate, Sodium Oleanolate, Sodium Oleoyl Lactylate, Sodium Oxalate, Sodium Pantetheine Sulfonate, Sodium Pantothenate, Sodium Paraben, Sodium PCA, Sodium PCA Methylsilanol, Sodium PEG-6 Cocamide Carboxylate, Sodium PEG-8 Cocamide Carboxylate, Sodium PEG-4 Cocamide Sulfate, Sodium PEG-50 Hydrogenated Castor Oil Succinate, Sodium PEG-3 Lauramide Carboxylate, Sodium PEG-4 Lauramide Carboxylate, Sodium PEG-7 Olive Oil Carboxylate, Sodium PEG-8 Palm Glycerides Carboxylate, Sodium PG-Sulfonate, Sodium Phenoxide, Sodium Phenylbenzimidazole Sulfonate, Sodium o-Phenylphenate, Sodium Phytate, Sodium Picramate, Sodium Polyaspartate, Sodium Propionate, Sodium Propoxy PPG-2 Acetate, Sodium Propylparaben, Sodium Pyrithione, Sodium Pyruvate, Sodium Riboflavin Phosphate, Sodium Saccharin, Sodium Salicylate, Sodium Sorbate, Sodium Stearoxy PG-Hydroxyethylcellulose Sulfonate, Sodium Stearoyl Hyaturonate, Sodium Stearoyl Lactylate, Sodium Stearyl Phthalamate, Sodium Succinate, Sodium Succinoyl Gelatin, Sodium Sucrose Octasulfate, Sodium Taurine Laurate, Sodium/TEA-Undecylenoyl Alginate, Sodium/TEA-Undecylenoyl Carrageenan, Sodium Thioglycolate, Sodium Trideceth-3 Carboxylate, Sodium Trideceth-4 Carboxylate, Sodium Trideceth-6 Carboxylate, Sodium Trideceth-7 Carboxylate, Sodium Trideceth-8 Carboxylate, Sodium Trideceth-12 Carboxylate, Sodium Trideceth-15 Carboxylate, Sodium Trideceth-19 Carboxylate, Sodium Undeceth-5 Carboxylate, Sodium Ursolate, Sodium Usnate, Sodium Zinc Cetyl Phosphate, Stearalkonium Dimethicone PEG-8 Phthalate, Strontium Acetate, Strontium Thioglycolate, TEA-Carbomer, TEA-Cocoyl Alaninate, TEA-EDTA, TEA-Glyceryl Dimaleate, TEA-Lauroyl Lactylate, TEA-Lauroyl/Myristoyl Aspartate, TEA-PEG-50 Hydrogenated Castor Oil Succinate, TEA-Phenylbenzimidazole Sulfonate, Tetrapotassium Etidronate, Tetrasodium EDTA, Tetrasodium Etidronate, Tetrasodium Glutamate Diacetate, Tetrasodium Iminodisuccinate, Thimerosal, Thurfylnicotinate HCI, Titanium Isostearates, Titanium Salicylate, Tripotassium EDTA, Trisodium Dicarboxymethyl Alaninate, Trisodium Fructose Diphosphate, Trisodium Glycyrrhizate, Trisodium Inositol Triphosphate, Zinc Acetate, Zinc Citrate, Zinc Cysteinate, Zinc Dibutyldithiocarbamate, Zinc Formaldehyde Sulfoxylate, Zinc Glucoheptonate, Zinc Gluconate, Zinc Glycyrrhetinate, Zinc Lactate, Zinc PCA, Zinc Picolinate, Zinc Pyrithione, Zinc Salicylate, and Zinc Thiosalicylate. Salts may also be formed of surfactants, preservatives, polymers, dyes, penetrants, proteins, peptides, or other components of compositions as described herein.

Typically the concentration of the salt in the final composition is in the range from about 1% to about 20% by weight. In certain embodiments, the concentration of the salt is about 5% to about 10% by weight. In certain embodiments, the concentration of the salt is about 2% to about 10% by weight. In certain embodiments, the concentration of the salt is about 2% to about 20% by weight. In certain embodiments, the concentration of the salt is about 5% to about 20% by weight. In certain embodiments, the concentration of the salt is about 2% to about 5% by weight. In certain embodiments, the concentration of the salt is about 0.5% to about 5% by weight. In certain embodiments, the concentration of the salt in the final composition is about 1%, about 2%, about 3%, about 4%, about 5%, about 10%, about 15%, about 20%, about 25%, or about 30% by weight.

### pH Adjusting Agents/Buffers/Neutralizing Agents

The inventive cosmetic compositions typically include pH adjusting agents, buffers, neutralizing agents, and the like. Such agents may be used to lower the pH, raise the pH, or maintain the pH of the final composition at a particular level. Exemplary pH buffering agents useful in the present invention include Aluminum Glycinate, Aluminum Lactate, Ammonium Acetate, Ammonium Carbonate, Ammonium Hexafluorophosphate, Ammonium Lactate, Ammonium Phosphate, Boric Acid, Calcium Carbonate, Calcium Phosphate, Cyclohexylamine, Diammonium Citrate, Diammonium Phosphate, Diethanolamine Bisulfate, Diethylamine, Diethyl Ethanolamine, Disodium Fumarate, Disodium Phosphate, Disodium Pyrophosphate, Ectoin, Ethanolamine HCI, Glycine, Hydroxyethylpiperazine Ethane Sulfonic Acid, Lauryl p-Cresol Ketoxime, Lithium Fluoride, Magnesium Acetate, M EA-Borate, MIPA-Borate, PEG-114 Methylether Polyepsilon Capralactone, Phosphonobutanetricarboxylic Acid, Potassium Acetate, Potassium Bicarbonate, Potassium Biphthalate, Potassium Citrate, Potassium Lactate, Sodium Acetate, Sodium Aluminate, Sodium Aluminum Lactate, Sodium Bicarbonate, Sodium Citrate, Sodium Fumarate, Sodium Lactate, Sodium Phosphate, Sodium Silicate, Sodium Succinate, Sodium Trimetaphosphate, Tetrapotassium Pyrophosphate, Tetrasodium Pyrophosphate, Urea, and Zinc Glycinate. Exemplary pH adjusting agents useful in accordance with the present invention include Acetic Acid, Acetyl Mandelic Acid, Adipic Acid, Aluminum Triformate, 2-Aminobutanol, Aminoethyl Propanediol, Aminomethyl Propanediol, Aminomethyl Propanol, Ammonia, Ammonium Bicarbonate, Ammonium Carbamate, Ammonium Carbonate, Ammonium Glycolate, Ammonium Hydroxide, Ammonium Phosphate, Ascorbic Acid, Azelaic Acid, Benzoic Acid, Bis-Hydroxyethyl Tromethamine, Calcium Citrate, Calcium Dihydrogen Phosphate, Calcium Hydroxide, Calcium Oxide, Citric Acid, Diethanolamine, Diethanolamine Bisulfate, Diisopropanolamine, Diisopropylamine, Dimethyl MEA, Dioleoyl Edetolmonium Methosulfate, Dipotassium Phosphate, Dipropylenetriamine, Disodium Fumarate, Disodium Phosphate, Disodium Pyrophosphate, Disodium Tartrate, Ethanolamine, Ethanolamine HCI, Formic Acid, Fumaric Acid, Galacturonic Acid, Glucoheptonic Acid, Glucosamine HCI, Glucuronic Acid, Glutaric Acid, Glycolic Acid, Glyoxylic Acid, Guanidine Carbonate, Hydrobromic Acid, Hydrochloric Acid, Imidazole, Isopropanolamine, Isopropylamine, Ketoglutaric Acid, Lactic Acid, Lactobionic Acid, Lithium Hydroxide, Magnesium Carbonate, Magnesium Carbonate Hydroxide, Magnesium Hydroxide, Magnesium Oxide, Maleic Acid, Malic Acid, Malonic Acid, Metaphosphoric Acid, Methylethanolamine, Methylglucamine, Mixed Isopropanolamines, Monosodium Citrate, Morpholine, Oxalic Acid, PEG-114 Methylether Polyepsilon Capralactone, Pentapotassium Triphosphate, Pentasodium Triphosphate, Phosphoric Acid, Potassium Bicarbonate, Potassium Biphthalate, Potassium Borate, Potassium Carbonate, Potassium Citrate, Potassium Hydroxide, Potassium Magnesium Aspartate, Potassium Phosphate, Potassium Tartrate, Propionic Acid, Quinic Acid, Ribonic Acid, Sebacic Acid, Sodium Aluminate, Sodium Bicarbonate, Sodium Bisulfate, Sodium Borate, Sodium Carbonate, Sodium Citrate, Sodium Fumarate, Sodium Hydroxide, Sodium Oxide, Sodium Sesquicarbonate, Sodium Silicate, Sodium Succinate, Sodium Trimetaphosphate, Strontium Hydroxide, Succinic Acid, Sulfuric Acid, Tartaric Acid, TEA-Diricinoleate/IPDI Copolymer, Tetrapotassium Pyrophosphate, Tetrasodium Pyrophosphate, Triethanolamine, Triisopropanolamine, Trisodium Phosphate, Tromethamine, Vinegar, Zinc Carbonate Hydroxide, Zinc Glycinate, and Zinc Magnesium Aspartate. The concentration of the pH adjusting agent, buffer, or neutralizing agent in the final composition is in the range from about 0.01% to about 10% by weight. In certain embodiments, the concentration of the agent is about 0.1% to about 10% by weight. In certain embodiments, the concentration of the agent is about 1% to about 10% by weight. In certain embodiments, the concentration of the agent is about 0.1% to about 5% by weight. In certain embodiments, the concentration of the agent is about 0.1% to about 3% by weight. In certain embodiments, the concentration of the agent is about 1% to about 5% by weight.

### Absorbents

Certain cosmetic compositions of the present invention include absorbents. In certain embodiments, the inventive cosmetic composition with an absorbent is a powder. Absorbents are typically ingredients with a large surface area which can attract other materials such as lipids. Exemplary absorbents useful in accordance with the present invention include acrylates/Bis-Hydroxypropyl Dimethicone, Crosspolymer, Acrylates Crosspolymer, Activated Clay, Alumina Magnesium Metasilicate, Aluminum Silicate, Aluminum Starch Octenylsuccinate, Ammonium Silver Zinc Aluminum Silicate, Amylodextrin, Attapulgite, Avena Sativa (Oat) Bran, Avena Sativa (Oat) Kernel Flour, Avena Sativa (Oat) Kernel Meal, Avena Sativa (Oat) Starch, Bentonite, Butyl Acrylate Crosspolymer, Calamine, Calcium Silicate, Calcium Starch Isododecenylsuccinate, Calcium Starch Octenylsuccinate, Carnauba Acid Wax, Cellulose, Chalk, Charcoal Powder, Citrus Aurantium Dulcis (Orange) Peel Powder, Citrus Grandis (Grapefruit) Peel Powder, Citrus Medica Limonum (Lemon) Peel Powder, Colloidal Oatmeal, Corn Starch Modified, Cyclodextrin, Dextrin, Diatomaceous Earth, Dimethylimidazolidinone Corn Starch, Dimethylimidazolidinone Rice Starch, DVB/Isobornyl Methacrylate/Lauryl Methacrylate Copolymer, Earthworm Conditioned Soil, Elguea Clay, Fuller's Earth, Alpha-Glucan, Glyceryl Starch, Hectorite, Helianthus Annuus (Sunflower) Seedcake, Hydrated Silica, Illite, Kaolin, Loess, Magnesium Aluminum Silicate, Magnesium Carbonate, Magnesium Hydroxide, Magnesium Oxide, Magnesium Silicate, Magnesium Trisilicate, Maltodextrin, Microcrystalline Cellulose, Montmorillonite, Moroccan Lava Clay, Nylon 6/12, Nylon-611, Oryza Sativa (Rice) Starch, Perfluoroheptane, Perfluoromethylcyclohexane, Perfluoromethyldecalin, Perlite, Phaseolus Angularis Seed Starch, Phonolite, Potassium Aluminum Polyacrylate, Pyrophyllite, Salt Mine Mud, Silica, Silica Dimethicone Silylate, Silicone Absorbents, Silt, Silver Copper Zeolite, Sodium Acrylates Crosspolymer, Sodium Magnesium Fluorosilicate, Sodium Naphthol Sulfonate, Sodium Polyacrylate, Sodium Polyacrylate Starch, Sodium Starch Octenylsuccinate, Solanum Tuberosum (Potato) Starch, Styrene/Stearyl Methacrylate Crosspolymer, Talc, Tanakura Clay, Triticum Vulgare (Wheat) Germ Powder, Triticum Vulgare (Wheat) Starch, Umber, Volcanic Ash, Wood Powder, Zea Mays (Corn) Seed Flour, Zea Mays (Corn) Starch, and Zeolite. The concentration of the absorbent in the final composition is in the range from about 1% to about 50% by weight. In certain embodiments, the concentration of absorbent is about 1% to about 20% by weight. In certain embodiments, the concentration of absorbent is about 1% to about 10% by weight. In certain embodiments, the concentration of absorbent is about 5% to about 15% by weight. In certain embodiments, the concentration of absorbent is about 10% to about 50% by weight. In certain embodiments, the concentration of absorbent is about 20% to about 50% by weight.

In certain embodiments, the cosmetically acceptable excipient or vehicle is a silicon-containing material. For example, a humectant, oil, lubricant, emollient, thickener, polymer, resin, film former, surfactant detergent, emulsifier, volatile, or absorbent may be a chemical compound that include silicon. In certain embodiments, a silicon-containing polymer is used in the cosmetic composition. In certain embodiments, a silicon-containing emulsifier, surfactant, or detergent is used in the cosmetic composition. In certain embodiments, a silicon-containing emollient is used in the cosmetic composition. In certain embodiments, a silicon-containing volatile is used in the cosmetic composition. Such silicon-containing materials may be used to enhance the feel, modify the surface characteristics, or improve the spreading or wetting properties of the final composition. Silicon-containing materials may also act to aid in the solubilization of the MMP inhibitor. Silicon-containing materials can be divided into several categories: volatiles (e.g., disiloxane, ethyltrisiloxane, cyclopentasiloxane, cyclohexasiloxane, PEG/PPG-20/15 dimethicone), polydimethylsiloxane (e.g., dimethicone), polydimethylsiloxane/dimethicone, gum fluid blends (e.g., cyclopentasiloxane and dimethiconol, cyclopentasiloxane and dimethicone, dimethicone and isododecane, dimethicone), phenyl modified fluids (e.g., phenyltrimethicone, bisphenylpropyl dimethicone, phenethyl dimethicone, phenylpropyldimethylsiloxysilicate), silicone waxes (e.g., cetearyl methicone, C₃₀₋₄₅ alkyl dimethicone), alkyl silicones (e.g., caprylyl methicone), polyether siloxanes (e.g., PEG-5/PPG-3 methicone, PEG-20/PPG-23 dimethicone, PEG-12 dimethicone, PEG-17 dimethicone, and PPG-12 dimethicone), aminofunctional silicones/amodimethicone (e.g., amodimethicone, bisamino PEG/PPG-41/3 aminoethyl PG-propyl dimethicone, polysilicone-18, polysilicone-18 cetyl phosphate, DEA PG-propyl PEG/PPG-18/21 dimethicone), silicone quats (e.g., silicone quaternium-18), silicone resins (e.g., trimethylsiloxysilicate, diisostearoyl trimethylolpropane siloxysilicate, silica dimethicone silylate), silicone spheres (e.g., polymethylsilsesquioxane), dimethicone/dimethiconol emulsions (e.g., dimethicone and laureth-4 and laureth-23, dimethiconol and sodium dodecylbenzenesulfonate, dimethiconol and TEA dodecylbenzenesulfonate), aminofunctional emulsions (e.g., amodimethicone and trideceth-12 and cetrimonium chloride, amodimethicone and trideceth-12 and glycerin and cetrimonium chloride, amodimethicone and C11-15 pareth-7 and laureth-9 and glycerin and trideceth-12, amodimethicone and C11-15 pareth-7 and laureth-9 and glycerin and trideceth-12), silicone polyacrylate emulsions (e.g., dimethicone PEG-8 polyacrylate), polyether-modified silicones (e.g., PEG-11 methyl ether dimethicone, PEG/PPG-20/22 butyl ether dimethicone, PEG-9 dimethicone, PEG-3 dimethicone, PEG-9 methyl ether dimethicone, PEG-10 dimethicone, PEG/PPG-10/3 oleyl ether dimethicone, PEG-9 polydimethylsioxyethyl dimethicone, lauryl PEG-9 polydimethylsiloxyethyl dimethicone, dimethicone and dimethicone/PEG-10/15 crosspolymer, mineral oil and PEG-15/lauryl dimethicone crosspolymer, isododecane and PEG-15/lauryl dimethicone crosspolymer, triethylhexanoin and PEG-15/lauryl dimethicone crosspolymer, squalane and PEG-10/lauryl dimethicone crosspolymer, PEG-15/lauryl dimethicone crosspolymer), polyglycerin-modified silicones (e.g., polyglyceryl-3 disiloxane dimethicone, polyglyceryl-3 polydimethylsiloxyethyl dimethicone, lauryl polyglyceryl-3 polydimethylsiloxyethyl dimethicone, dimethicone and dimethicone/polyglycerin-3 crosspolymer, mineral oil and lauryl dimethicone/polyglycerin-3 crosspolymer, isododecane and lauryl dimethicone/polyglycerin-3 crosspolymer, triethylhexanoin and lauryl dimethicone/polyglycerin-3 crosspolymer, squalane and lauryl dimethicone/polyglycerin-3 crosspolymer). Any of the silicon-containing materials may be used alone or in combination with other materials. Examples of suppliers of silicon-containing materials include General Electric, Dow Corning, and Shin Etsu.

In certain embodiments, the cosmetic composition is a lotion comprising the following ingredients: water, disodium EDTA, CARBOPOL 981 REGULAR (carbomer), KELTROL CG-SFT (xantham gum), CETIOL HE (PEG-7 glyceryl cocoate), glycerin, TENOX BHT (butylated hydroxytoluene), ARLASOLVE DMI (dimethyl isosorbide), LIPOWAX D (ceteraryl alcohol ceteareth-20), LIPONATE GC (capryl/capric triglyceride), LIPOMULSE 165 (glyceryl stearate PEG-100 stearate), sodium hydroxide, germazide PMP (phenoxyethanol, chlorphenesin, methylparaben, propylparaben), and an MMP inhibitor. In certain embodiments, the MMP inhibitor is an MMP inhibitor described herein. In certain particular embodiments, the MMP inhibitor is of formula:

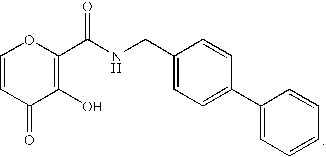

In certain embodiments, the ingredients are present in the composition at the following percentages by weight:

| | |
|---|---|
| Water | 45.0-70.165% |
| Disodium EDTA | 0.05-0.15% |
| CARBOPOL 981 REGULAR (carbomer) | 7.50-12.0% |
| KELTROL CG-SFT (xantham gum) | 0.05-1.00% |
| CETIOL HE (PEG-7 glyceryl cocoate) | 3.50-7.50% |
| Glycerin | 0.25-2.00% |
| TENOX BHT (butylated hydroxytoluene) | 0.05-0.015% |
| ARLASOLVE DMI (dimethyl isosorbide) | 25.00% |
| LIPOWAX D (ceteraryl alcohol ceteareth-20) | 0.50-3.00% |
| LIPONATE GC (capryl/capric triglyceride) | 5.00-10.0% |
| LIPOMULSE 165 | 3.00-6.00% |
| (glyceryl stearate PEG-100 stearate) | |
| Sodium Hydroxide | 0.20-1.50% |
| GERMAZIDE PMP (phenoxyethanol, | 0.75-2.00% |
| chlorphenesin, methylparaben, propylparaben) | |
| MMP Inhibitor | 0.10% |

The composition is formulated by first dispersing CARBOPOL 981 REGULAR (carbomer) and KELTROL CG-SFT (xantham gum) in water then adding EDTA and heating to 70° C. CETIOL HE and glycerin is then added to produce phase A. Phase B is prepared by mixing TENOX BHT (butylated hydroxytoluene), ARLASOLVE DMI (dimethyl isosorbide), LIPOWAX D (ceteraryl alcohol ceteareth-20), LIPONATE GC (capryl/capric triglyceride), LIPOMULSE 165 (glyceryl stearate PEG-100 stearate), and the MMP inhibitor together and heated to 65-70° C. Phases A and B are then mixed together at 65-70° C. until a uniform mixture is obtained. The mixture is then cooled to 60° C. and sodium hydroxide is added. The resulting mixture is mixed until uniform and then cooled to 40° C. Finally GERMAZIDE PMP is added to the mixture and combined until uniform to complete the formulation.

In certain embodiments, the cosmetic composition comprises the following ingredients: water, disodium EDTA, KELTROL CG-SFT (xantham gum), CARBOPOL (carbomer), ACCONON CC-6 (PEG-6 caprylic/capric glycerides), glycerin, TENOX BHT (butylated hydroxytoluene), ARLASOLVE DMI (dimethyl isosorbide), LIPOWAX D (ceteraryl alcohol ceteareth-20), LIPONATE GC (capryl/capric triglyceride), LIPOMULSE 165 (glyceryl stearate PEG-100 stearate), sodium hydroxide, germazide PMP (phenoxyethanol, chlorphenesin, methylparaben, propylparaben), and an MMP inhibitor. Vitamins such as vitamin E and vitamin C are optionally included in the formulation. In certain embodiments, the MMP inhibitor is an MMP inhibitor described herein. In certain particular embodiments, the MMP inhibitor is of the formula:

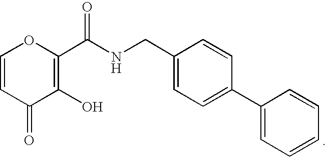

In certain embodiments, the ingredient are present in the composition at the following percentages by weight:

| | |
|---|---|
| Water | 41.8% |
| Disodium EDTA | 0.05% |
| CARBOPOL 981 REGULAR (carbomer) | 10.0% |
| KELTROL CG-SFT (xantham gum) | 0.5% |
| ACCONON CC-6 (PEG-6 caprylic/capric glycerides) | 15.0% |
| Glycerin | 1.0% |
| MMP inhibitor | 0.04% |
| TENOX BHT (butylated hydroxytoluene) | 0.6% |
| 0.2% MMP inhibitor in ARLASOLVE DMI (dimethyl | 5.00% |
| isosorbide) | |
| LIPOWAX D (ceteraryl alcohol ceteareth-20) | 12.0% |
| LIPONATE GC (capryl/capric triglyceride) | 5.0% |
| LIPOMULSE 165 (glyceryl stearate PEG-100 stearate) | 6.00% |
| 20% Sodium Hydroxide | 1.0% |
| GERMAZIDE PMP (phenoxyethanol, | 2.00% |
| chlorphenesin, methylparaben, propylparaben) | |

The total concentration of MMP inhibitor in the composition is approximately 0.05%. The concentration of the MMP inhibitor is dependent on the concentration of the emulsifier (ACCONON CC-6). The concentration of ACCONON CC-6 may range from 15% to 50%, thereby increasing the CARBOPOL concentration and reducing the LIPOWAX D and LIPOMULSE concentrations. The use of the emulsifier ACCONON CC-6 allows for the use of lower concentrations of dimethylisosorbide. The emulsifier is thought to enhance the aqueous solubility of the MMP inhibitor.

The composition is formulated by first mixing ACCONON CC-6 with water. The MMP inhibitor is added to the resulting mixture and allowed to stir for 24 hours at room temperature to solubilize the inhibitor. The CARBOPOL and xanthum gum is dispersed in water and EDTA is added. The mixture is heated to 70° C., and glycerin is added to form phase A. The BHT, vitamin E, vitamin C, MMP inhibitor in DMI, LIPOWAX D, LIPOMATE GC, and LIPOMULSE 165 are mixed together and heated to 65-70° C. to form phase B. Phase B is added to phase A and mixed at 65-70° C. until uniform. The mixture is cooled to 60° C., and 20% NaOH is added until a pH of 6.4-7.0 is achieved. The mixture is further cooled to 40° C. and the germazide is added to complete the formulation.

### Pharmaceutical Compositions

The present invention provides novel pharmaceutical compositions comprising a therapeutically effective amount of a MMP inhibitor and at least one pharmaceutically acceptable excipient. In some embodiments, the present invention provides for pharmaceutical compositions comprising an MMP inhibitor as described herein. Such pharmaceutical compositions may optionally comprise one or more additional biologically active substances. In accordance with some embodiments, a method of administering a pharmaceutical composition to a subject in need thereof is provided. In some embodiments, inventive compositions are administered to a human. For the purposes of the present invention, the phrase “active ingredient” generally refers to an MMP inhibitor.

Although the descriptions of pharmaceutical compositions provided herein are principally directed to pharmaceutical compositions which are suitable for administration to humans, it will be understood by the skilled artisan that such compositions are generally suitable for administration to animals of all sorts.

The formulations of the pharmaceutical compositions described herein may be prepared by any method known or hereafter developed in the art of pharmaceutics. In general, such preparatory methods include the step of bringing the active ingredient into association with one or more excipients, and then, if necessary or desirable, shaping or packaging the product into a desired single- or multi-dose unit.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable excipient(s), and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical formulations of the present invention may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by the United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include antioxidants, chelating agents, antimicrobial preservatives, antifungal preservatives, alcohol preservatives, acidic preservatives, and other preservatives as described herein. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the targeted particles of the invention are mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the targeted particles of this invention with suitable non-irritating excipients such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

The active ingredients can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a targeted particle of this invention may include ointments, pastes, creams, lotions, gels, powders, solutions, sprays, inhalants and/or patches. Generally, the active component is admixed under sterile conditions with a pharmaceutically acceptable excipient and/or any needed preservatives and/or buffers as may be required. Additionally, the present invention contemplates the use of transdermal patches, which often have the added advantage of providing controlled delivery of an active ingredient to the body. Such dosage forms may be prepared, for example, by dissolving and/or dispensing the active ingredient in the proper medium. Alternatively or additionally, the rate may be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate compositions in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi-liquid preparations such as liniments, lotions, oil-in-water, and/or water-in-oil emulsions such as creams, ointments, pastes, solutions, and suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 μm to about 7 μm or from about 1 μm to about 6 μm. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 μm and at least 95% of the particles by number have a diameter less than 7 μm. Alternatively, at least 95% of the particles by weight have a diameter greater than 1 μm and at least 90% of the particles by number have a diameter less than 6 μm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions of the invention formulated for pulmonary delivery may provide the active ingredient in the form of droplets of a solution and/or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 μm to about 200 μm.

The formulations described herein as being useful for pulmonary delivery are useful for intranasal delivery of a pharmaceutical composition of the invention. Another formulation suitable for intranasal administration is a coarse powder comprising the active ingredient and having an average particle from about 0.2 to 500 micrometers. Such a formulation is administered in the manner in which snuff is taken, i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may comprise one or more of the additional ingredients described herein. A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder, or an aerosolized or atomized solution or suspension comprising the active ingredient. Such powdered, aerosolized, or aerosolized formulations, when dispersed, may have an average particle and/or droplet size in the range from about 0.1 to about 200 nanometers, and may further comprise one or more of the additional ingredients described herein.

A pharmaceutical composition of the invention may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are contemplated as being within the scope of this invention.

General considerations in the formulation and/or manufacture of pharmaceutical agents may be found, for example, in Remington: The Science and Practice of Pharmacy 21^{st} ed., Lippincott Williams & Wilkins, 2005.

For pharmaceutical uses, the MMP inhibitor may be delivered using a patch, by injection, using a microneedle delivery system, using iontophoresis, using electroporation, or using ultrasound. In certain embodiments, iontophoresis is used to deliver the MMP inhibitor to the skin of a subject. Inotophoresis is described in U.S. Published Patent Application 2007/0260170, which is incorporated herein by reference. In certain embodiments, electroporation is used to deliver the MMP inhibitor to the skin of a subject. Electroporation as a means for administering a pharmaceutical to the skin is described in U.S. Published Patent Application 2008/0058706, which is incorporated herein by reference. In certain embodiments, ultrasound is used to administer an MMP inhibitor to the skin. See U.S. Pat. No. 6,002,961, which is incorporated herein by reference. Microneedle systems for delivering a pharmaceutical to the skin may also be used to deliver an MMP inhibitor. See U.S. Published Patent Application 2007/01616964, which is incorporated herein by reference.

### Uses

The inventive cosmetic compositions with the MMP inhibitors are used to care for, avoid, or prevent the signs and/or appearance of aged or sun-damaged skin. In certain embodiments, the composition is applied to skin in order to prevent these signs before they occur. For example, the composition may be applied to skin to prevent or reduce the appearance of the following cosmetic attributes: coarse wrinkles, fine wrinkles, lines, sagging, pigmentation changes, mottled hyperpigmentation, lentigines, tactile roughness, telangiectasia, pore size, elastosis, laxity, redness, etc. In other embodiments, the composition is used to reverse the signs of aging or sun damage. In certain embodiments, the composition is used to improve the appearance of aging or sun damaged skin. The inventive cosmetic compositions may be applied to any part of the body of a subject. The subject is typically a human. In certain embodiments, the cosmetic composition is applied to the face and/or neck. In other embodiments, the composition is applied to the arms, legs, feet, hands, chest, or back of the subject. The composition may be applied once or applied repeatedly to a subject. In certain embodiments, the composition is applied repeatedly to an area to prevent or treat the signs of aging or sun damage or to improve the appearance of aging or sun damaged skin. For example, in certain embodiments, the composition is applied once a day to an area to be treated. In other embodiments, the composition is applied multiple times per day (e.g., 2-5 times per day). In certain embodiments, the composition is applied every other day, every third day, or every fourth day. In certain embodiments, the composition is applied once, twice, or three times per week. In certain embodiments, the composition is applied to the area to be treated every other week. In certain embodiments, the composition is applied once per month. As would be appreciated by one of skill in this art, the administration of the composition will depend on the patient, the skin condition, the area of the body being treated, the MMP inhibitor being used, the components of the composition, etc.

Changes in the skin as a result of the use of the inventive composition may be measured using any number of techniques known in the art. In certain embodiments, improvements are based the Physician Global Assessment (PGA) of skin appearance based on the Griffiths scale (0-8). Griffiths et al., Arch. Dermatol. Res. 128:347-51, 1992; incorporated herein by reference. In certain embodiments, one or more skin characteristics may be assessed on a scale from 0 to 9. Such characteristics as fine wrinkling, mottled hyperpigmentation, lentigines, tactile roughness, coarse wrinkling, telangiectasia, pore size, elastosis, and laxity may be measured. In certain embodiments, the Physician Forced Choice (PFC) Preference based on digital images of skin treated with the inventive composition is used to assess the effect of the inventive compositions. In certain embodiments, silicon replicas of the skin may be used to assess the inventive compositions. In certain embodiments, a patient's assessment of their own skin may be used.

The inventive pharmaceutical compositions with the MMP inhibitors are used to treat or prevent any disease, disorder, or condition. In certain embodiments, the composition is applied to skin. For example, the disease being treated may be a skin disease. In certain embodiments, the skin disease is an inflammatory disease. In certain embodiments, the skin disease is an autoimmune disease. In certain embodiments, the composition is used to promote wound healing. In certain embodiments, the skin disease is a proliferative disease. In certain particular embodiments, the skin disease is skin cancer. The subject is typically a human; however, other essentially hairless mammals (e.g., hairless rodents, dogs, or cats) may also be treated using the inventive compositions. The composition may be applied once or applied repeatedly to a subject. In certain embodiments, the composition is applied repeatedly to an affected area. For example, in certain embodiments, the composition is applied once a day to an area to be treated. In other embodiments, the composition is applied multiple times per day (e.g., 2-5 times per day). In certain embodiments, the composition is applied every other day, every third day, or every fourth day. In certain embodiments, the composition is applied once, twice, or three times per week. In certain embodiments, the composition is applied to the area to be treated every other week. In certain embodiments, the composition is applied once per month. In certain embodiments, the administration of the composition is continued until the desired response is achieved. As would be appreciated by one of skill in this art, the administration of the composition will depend on the patient, the condition being treated or prevented, the MMP inhibitor being used, the components of the composition, etc.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Moisturizing Facial Lotion

A thick moisturizing facial lotion is prepared with the following ingredients from the indicated suppliers at the listed percentages:

| Ingredient | Tradename | Supplier | % by weight |
|---|---|---|---|
| water | N/A | | 45.0-70.165% |
| disodium EDTA | N/A | AKZO | 0.05-0.15% |
| carbomer | CARBOPOL 981 | Noveon | 7.50-12.0% |
| | REGULAR | | |
| xantham gum | KELTROL CG-SFT | KELCO | 0.05-1.00% |
| PEG-7 glyceryl | CETIOL HE | Cogins | 3.50-7.50% |
| cocoate | | | |
| Glycerin | N/A | Acme Hardesty | 0.25-2.00% |
| butylated | TENOX BHT | Eastman | 0.05-0.015% |
| hydroxytoluene | | | |
| dimethyl isosorbide | ARLASOLVE DMI | Uniqema | 25.00% |
| ceteraryl alcohol | LIPOWAX D | LIPO | 0.50-3.00% |
| ceteareth-20 | | | |
| capryl/capric | LIPONATE GC | LIPO | 5.00-10.0% |
| triglyceride | | | |
| glyceryl stearate | LIPOMULSE 165 | LIPO | 3.00-6.00% |
| PEG-100 stearate | | | |
| sodium hydroxide | N/A | Chemtech Inc. | 0.20-1.50% |
| phenoxyethanol, | GERMAZIDE PMP | BASF | 0.75-2.00% |
| chlorphenesin, | | | |
| methylparaben, | | | |
| propylparaben | | | |
| MMP Inhibitor | | | 0.10% |
| 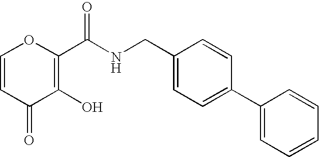 | | | |

The lotion is formulated by first dispersing the CARBOPOL 981 REGULAR (carbomer) and KELTROL CG-SFT (xantham gum) in water then adding EDTA and heating to 70° C. CETIOL HE and glycerin is then added to produce phase A. Phase B is prepared by mixing TENOX BHT (butylated hydroxytoluene), ARLASOLVE DMI (dimethyl isosorbide), LIPOWAX D (ceteraryl alcohol ceteareth-20), LIPONATE GC (capryl/capric triglyceride), LIPOMULSE 165 (glyceryl stearate PEG-100 stearate), and the MMP inhibitor together and heated to 65-70° C. Phases A and B are then mixed together at 65-70° C. until a uniform mixture is obtained. The mixture is then cooled to 60° C. and sodium hydroxide is added. The resulting mixture is mixed until uniform and then cooled to 40° C. Finally GERMAZIDE PMP is added to the mixture and combined until uniform to complete the formulation of the lotion.

### Example 2

### Another Lotion

A lotion is prepared with the following ingredients at the listed percentages:

| Ingredient | Tradename | % by weight |
|---|---|---|
| water | N/A | 41.81% |
| disodium EDTA | N/A | 0.05-0.15% |
| carbomer | CARBOPOL 981 | 10.00% |
| | REGULAR | |
| xantham gum | KELTROL CG-SFT | 0.5% |
| PEG-6 caprylic/capric | ACCONON CC-6 | 15.0% |
| glycerides | | |
| Glycerin | N/A | 1.0% |
| MMP Inhibitor | | 0.04% |
| 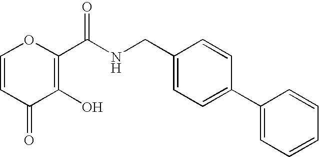 | | |
| butylated hydroxytoluene, | TENOX BHT | 0.6% |
| vitamin E, vitamin C | | |
| 0.2% MMP Inhibitor | ARLASOLVE DMI | 5.0% |
| 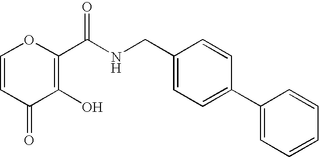 | | |
| in dimethyl isosorbide | | |
| (DMI) | | |
| ceteraryl alcohol | LIPOWAX D | 12.0% |
| ceteareth-20 | | |
| capryl/capric triglyceride | LIPONATE GC | 5.0% |
| glyceryl stearate PEG-100 | LIPOMULSE 165 | 6.0% |
| stearate | | |
| 20% sodium hydroxide | N/A | 1.0% |
| phenoxyethanol, | GERMAZIDE PMP | 2.0% |
| chlorphenesin, | | |
| methylparaben, | | |
| propylparaben | | |

The lotion is formulated by first mixing ACCONON CC-6 with water. The MMP inhibitor,

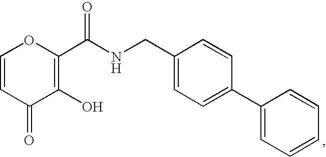

is added to the resulting mixture and allowed to stir for 24 hours at room temperature in order to solubilize the inhibitor. The CARBOPOL and KETROL is dispersed in water and EDTA is added. The mixture is heated to 70° C., and glycerin is added to form phase A. The BHT, vitamin E, vitamin C, the MMP inhibitor in DMI, LIPOWAX D, LIPOMATE GC, and LIPOMULSE 165 are mixed together and heated to 65-70° C. to form phase B. Phase B is added to phase A and mixed at 65-70° C. until uniform. The resulting mixture is cooled to 60° C., and 20% NaOH is added until a pH of 6.4-7.0 is achieved. The mixture is further cooled to 40° C., and the germazide is added to complete the formulation of the lotion.

### Example 3

### Topical Skin Cream

A topical skin cream is prepared with the following ingredients at the listed percentages:

| Ingredient | % by weight |
|---|---|
| water | Balance |
| Sodium lauryl sulfate | 2.5% |
| Quaternium-15 | 0.1% |
| Propyl paraben | 0.02% |
| Methyl paraben | 0.2% |
| Glycerin | 10% |
| Cetyl alcohol | 4.0% |
| Stearyl alcohol | 10% |
| Cetyl esters wax | 8.4% |
| Vitamin E acetate | 0.5% |
| Vitamin A acetate | 1.0% |
| MMP Inhibitor | 0.05-1.0% |

### Example 4

### Skin Care Cream and Lotions

A skin care cream or lotion containing an MMP inhibitor may be formulated with water, mink oil, vitamin E, vitamin A, ginseng, aloe vera, glycerin, lanolin, gotu kola, soybean oil, fish liver oil, hydrolyzed animal protein, dl-alpha tocopherol acetate, stearic acid, cetyl alcohol, citric acid, silicon, isopropylmyristate, propylene glycol, stearyl alcohol, glycerol stearate, dimethicone, lactic acid, quaternium-15, propylparaben, carbomer 934 and 940, triethanolamine, methylparaben, tetrasodium EDTA, DMDM hydantoin, diazolidinyl urea, and fragrance.

### Example 5

### Topical Skin Cream

A topical skin cream is prepared with the following ingredients at the listed percentages:

| Ingredient | % by weight |
|---|---|
| water | Balance |
| 1,3-butylene glycol | 2.0% |
| Methylparaben | 0.2% |
| Potassium sulfate | 1.0% |
| Isopropyl myristate | 2.0% |
| Liquid paraffin | 8.0% |
| Aluminum tristearate | 0.08% |
| Diglyceryl monooleate | 4.0% |
| MMP Inhibitor | 0.05-0.5% |
| Water | Balance |
| Diglyceryl monoisostearate | 5.0% |
| Aluminum tristearate | 0.08% |
| Liquid paraffin | 6.0% |
| Isopropyl myristate | 2.0% |
| Magnesium sulfate | 1.0% |
| Methyl paraben | 0.2% |
| Propylene glycol | 3.0% |
| MMP Inhibitor | 0.05-0.5% |
| Water | Balance |
| Propylene glycol | 3.0% |
| methylpolysiloxane | 0.2% |
| Magnesium sulfate | 1.0% |
| Diisopropyl sebacate | 2.0% |
| Squalene | 8.0% |
| Aluminum tristearate | 0.08% |
| Diglyceryl monooleate | 5.0% |
| MMP Inhibitor | 0.05-0.5% |

### Example 6

### High Friction Cosmetic Cream

A high friction cosmetic cream is prepared with the following ingredients at the listed percentages:

| Ingredient | % by weight |
|---|---|
| Water | Balance |
| Stearic acid | 12-15% |
| Sodium cetearyl sulfate | 0.5-1.5% |
| Myrj 59 | 1.5-2.0% |
| Span 60 | 1.5-2.0% |
| Parsol 1789 | 0-0.5% |
| Propyl paraben | ~0.1% |
| BHT | ~0.1% |
| Parsol MCX | 0-0.75% |
| Dimethicone | 0-1% |
| EDTA | 0.04% |
| Pamulen TR 2 | 0-0.05% |
| Methyl paraben | 0.15% |
| Humectant | 5-10% |
| MMP Inhibitor | 0.05-0.1% |

### Example 7

### Cosmetic Cream

A cosmetic cream is prepared with the following ingredients at the listed percentages:

| Ingredient | % by weight |
|---|---|
| Water | Balance |
| Stearic acid | 15-20% |
| Sodium cetearyl sulfate | 0.5-3% |
| Myrj 59 | 0-2.0% |
| Span 60 | 0-2.0% |
| Glycerin | 1-5% |
| MMP Inhibitor | 0.05-0.1% |

### Example 8

### Cosmetic Cream

A typical cosmetic cream is prepared with the following ingredients at the listed percentages:

| Ingredient | % by weight |
|---|---|
| Water | 69% |
| Disodium EDTA | 0.05% |
| Magnesium aluminum silicate | 0.6% |
| Methyl paraben | 0.15% |
| Simethicone | 0.01% |
| 1,3-butylene glycol | 3.0% |
| Hydroxyethylcellulose | 0.5% |
| Glycerin | 2.0% |
| Xantham gum | 0.2% |
| Triethanolamine | 1.2% |
| Stearic acid | 3.0% |
| Propyl paraben | 0.1% |
| Glyceryl hydroxystearate | 1.5% |
| Stearyl alcohol | 1.5% |
| Isostearyl palmitate | 6.0% |
| C₁₂₋₁₅ alcohols octanoate | 3.0% |
| Dimethicone | 1.0% |
| Cholesterol | 0.5% |
| Sorbitan stearate | 1.0% |
| Butylated hydroxytoluene (BHT) | 0.05% |
| Tocopheryl acetate | 0.1% |
| PEG-100 stearate | 2.0% |
| Sodium stearoyl lactylate | 0.5% |
| Hydroxycaprylic acid | 0.1% |
| Ammonium hydroxide | 2.4% |
| Alpha-bisabolol | 0.2% |
| MMP Inhibitor | 0.05-1.0% |

### Example 9

### Cosmetic Cream with Herbal Extracts

A cosmetic cream with herbal extracts is prepared with the following ingredients at the listed percentages:

| Ingredient | % by weight |
|---|---|
| Water | 50-90% |
| Sodium lauryl sulfate (30%) | 0.5-2.5% |
| Propylene glycol | 2.0-9.0% |
| Tetrasodium EDTA | 0.05-0.5% |
| Lanolin oil | 5.0-15.0% |
| Cetyl alcohol | 3.0-10.0% |
| Stearyl alcohol | 1.0-5.0% |
| Beeswax | 0.5-2.5% |
| Cod Liver Oil | 1.0-7.0% |
| BHT | 0.10-1.00% |
| St. John's Wort Extract | 0.05-0.5% |
| Witch Hazel Extract | 0.05-0.5% |
| Chamomile Extract | 0.05-0.5% |
| Arnica Extract | 0.05-0.5% |
| Methyl paraben | 0.1-0.5% |
| Propyl paraben | 0.1-0.5% |
| Fragrance | 0.05-0.5% |
| MMP Inhibitor | 0.05-1% |

### Example 10

### Cosmetic Cream with Lactylate Emulsifiers

A cosmetic cream with lactylate emulsifiers is prepared with the following ingredients at the listed percentages:

| Ingredient | % by weight |
|---|---|
| Water | 50-90% |
| Propylene glycol | 2-9% |
| Citric acid | 0.05-0.5% |
| Sodium stearoyl lactylate | 0.3-3% |
| Sodium isostearoyl lactylate | 0.05-1% |
| Tetrasodium EDTA | 0.05-0.25% |
| Lanolin oil | 5-15% |
| Cetyl alcohol | 1-8% |
| Cod liver oil | 1-7% |
| BHT | 0.1-1% |
| Methyl paraben | 0.1-0.5% |
| Propyl paraben | 0.1-0.5% |
| Fragrance | 0.05-0.5% |
| MMP Inhibitor | 0.05-1% |

### Example 11

### Cosmetic Cream with Carboxypolymethylene Polymer

A cosmetic cream with lactylate emulsifiers is prepared with the following ingredients at the listed percentages:

| Ingredient | % by weight |
|---|---|
| Water | 50-90% |
| Carboxypolymethylene polymer | 0.4-3% |
| Propylene glycol | 2-9% |
| Lanolin oil | 5-15% |
| Cetyl alcohol | 1-8% |
| Cod liver oil | 1-7% |
| BHT | 0.1-1% |
| Methyl paraben | 0.1-0.5% |
| Propyl paraben | 0.1-0.5% |
| Fragrance | 0.05-0.5% |
| Triethanolamine | 0.05-3% |
| MMP Inhibitor | 0.05-1% |

### Example 12

### Nonionic Oil in Water Emulsion

A thick moisturizing facial lotion is prepared with the following ingredients from the indicated suppliers at the listed percentages:

| Phase | Ingredient | Tradename | Supplier | % by weight |
|---|---|---|---|---|
| A | Water | N/A | | 45.0-70.165 |
| | Disodium EDTA | N/A | AKZO | 0.05-0.15 |
| | Carbomer | CARBOPOL 981 | Noveon | 7.50-12.0 |
| | | REGULAR | | |
| | Xantham gum | KELTROL CG-SFT | KELCO | 0.05-1.00 |
| | Carbomer | ULTREZ 10 | Noveon | 0-0.25 |
| | Acrylates/C10-30 | PEMULEN TR-2 | Noveon | 0-0.25 |
| | alkyl acrylate | | | |
| | crosspolymer | | | |
| | PEG-7 glyceryl | CETIOL HE | Cogins | 3.50-7.50 |
| | cocoate | | | |
| | Glycerin | N/A | Acme Hardesty | 0.25-2.00 |
| | Butylated | TENOX BHT | Eastman | 0.05-0.015 |
| | hydroxytoluene | | | |
| B | Ceteraryl alcohol | LIPOWAX D | LIPO | 0.50-3.00 |
| | ceteareth-20 | | | |
| | Capryl/capric | LIPONATE GC | LIPO | 5.00-10.0 |
| | triglyceride | | | |
| | Glyceryl stearate | LIPOMULSE 165 | LIPO | 3.00-6.0 |
| | PEG-100 stearate | | | |
| | Arlacel 80 (Span | SORBITAN OLEATE | Uniqema | 0-0.15 |
| | 80) | | | |
| | Dimethyl | ARLASOLVE DMI | Uniqema | 24.50 |
| | isosorbide | | | |
| | MMP Inhibitor | | | 0.01-0.50 |
| | 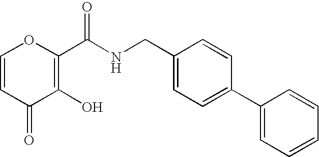 | | | |
| | BHT | BHT | | 0.20 |
| | Ascorbyl Palmitate | Vitamin C Palmitate | | 0.20 |
| | Tocopherol | Vitamin E | | 0.20 |
| C | Sodium hydroxide | N/A | Chemtech Inc. | 0.10-1.50 |
| D | Phenoxyethanol, | GERMAZIDE PMP | BASF | 0.75-2.00 |
| | chlorphenesin, | | | |
| | methylparaben, | | | |
| | propylparaben | | | |
| E | Sodium | RHEOCARE ATH | Seaview | 0-3.00 |
| | polyacrylate and | | Technologies | |
| | ethylhexyl stearate | | | |
| | and trideceth-6 | | | |

The lotion is formulated by first dispersing the CARBOPOL 981 REGULAR (carbomer) and KELTROL CG-SFT (xantham gum) in water, then adding EDTA and heating to 70° C. CETIOL HE and glycerin is then added to produce phase A. Phase B is prepared by mixing TENOX BHT (butylated hydroxytoluene), ARLASOLVE DMI (dimethyl isosorbide), LIPOWAX D (ceteraryl alcohol ceteareth-20), LIPONATE GC (capryl/capric triglyceride), LIPOMULSE 165 (glyceryl stearate PEG-100 stearate), and the MMP inhibitor together, and heating the mixture to 65-70° C. Phases A and B are then mixed together at 65-70° C. until a uniform mixture is obtained. The mixture is then cooled to 60° C., and sodium hydroxide is added. The resulting mixture is mixed until uniform and then cooled to 40° C. Finally GERMAZIDE PMP is added to the mixture and combined until uniform to complete the formulation of the lotion.

### Example 13

### Nonionic Water in Oil Emulsion

An emollient cream is prepared with the following ingredients at the listed percentages:

| Phase | Ingredient | Tradename | % by weight |
|---|---|---|---|
| A | Safflower Oil | | 20.0-35.0% |
| | Microcrystalline Wax | | 1.0-4.0% |
| | Shea Butter | | 0.0-8.0% |
| | Polyglyceryl-2-dioleate | | 3.0-8.0% |
| B | butylated hydroxytoluene, | TENOX BHT, | 0.3% |
| | vitamin E, vitamin C | | |
| | Palmitate | Tocopherol, | |
| | | Ascorbyl Palmitate | |
| | 0.2% MMP Inhibitor | | 0.01-0.50 |
| | 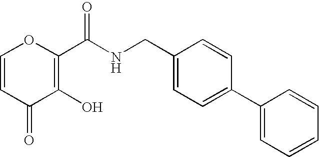 | | |
| | PEG 4 | | 5.0-20.0% |
| C | Water | | 13.0% |
| | L-Sodium Glutamate | | 0.1-1.8% |
| | L-Serine | | 0.1-0.8% |
| | disodium EDTA | | 0.05-0.15% |
| | | | 3.0-8.0% |
| D | Water | | q.s. |
| | Glycerin | N/A | 0-6.0% |
| E | phenoxyethanol, | GERMAZIDE PMP | 0.25-1.0% |
| | chlorphenesin, | | |
| | methylparaben, | | |
| | propylparaben | | |

The emollient cream is formulated by first mixing Phase B, the MMP inhibitor, in PEG-4 for 24 hours to solubilize. BHT, Vitamin E, and Vitamin C are added to complete the preparation of Phase B. Phase A and C are blended individually, and all three phases are heated to 70° C. Phase B is blended with Phase A, and the resulting mixture is slowly added to Phase C until the emulsion inverts. Phases D and E are blended individually. Phases D and E are added to the mixture. The resulting mixture is cooled to 30° C. and homogenized.

### Example 14

### Anionic Oil in Water Cosmetic Cream with Lactylate Emulsifiers

A cosmetic cream with lactylate emulsifiers is prepared with the following ingredients at the listed percentages:

| Ingredient | % by weight |
|---|---|
| Water | 50-90% |
| Glycerin | 2-9% |
| Citric acid | 0.05-0.5% |
| Sodium stearoyl lactylate | 0.3-3% |
| Sodium isostearoyl lactylate | 0.05-1% |
| Tetrasodium EDTA | 0.05-0.25% |
| Sunflower Seed oil | 5.0-15.0% |
| Cetyl alcohol | 1.0-8.0% |
| Cod liver oil | 1.0-7.0% |
| BHT | 0.1-1.0% |
| Methyl paraben | 0.1-0.5% |
| Propyl paraben | 0.1-0.5% |
| Fragrance | 0.05-0.5% |
| MMP Inhibitor | 0.05-1.0% |
| PEG-4 | 5.0-20.0% |
| butylated hydroxytoluene, vitamin | 0.3% |
| E, vitamin C Palmitate | |

### Example 15

### Another Cosmetic Cream w/MMP Inhibitor Suspended

A cosmetic cream is prepared with the following ingredients at the listed percentages:

| Ingredient | % by weight |
|---|---|
| Water | Balance |
| Stearic acid | 15-20% |
| Sodium cetearyl sulfate | 0.5-3% |
| Myrj 59 | 0-2.0% |
| Span 60 | 0-2.0% |
| Glycerin | 1-5% |
| MMP Inhibitor | 0.01-0.50 |
| butylated hydroxytoluene, | 0.3% |
| vitamin E, vitamin C palmitate | |

### Example 16

### Another Anionic Oil in Water Emulsion with UV Protection

A high friction cosmetic cream is prepared with the following ingredients at the listed percentages:

| Ingredient | % by weight |
|---|---|
| Water | Balance |
| Stearic acid | 12-15% |
| Sodium cetearyl sulfate | 0.5-1.5% |
| Myrj 59 | 1.5-2.0% |
| Span 60 | 1.5-2.0% |
| Parsol 1789 | 0-0.5% |
| Propyl paraben | ~0.1% |
| BHT | ~0.1% |
| Parsol MCX | 2.0% |
| Parsol 1789 | 2.0% |
| Benzophenone-3 | 2.0% |
| Dimethicone | 0-1% |
| EDTA | 0.04% |
| Pamulen TR 2 | 0-0.05% |
| Methyl paraben | 0.15% |
| Humectant | 5-10% |
| MMP Inhibitor | 0.01-0.50 |
| butylated hydroxytoluene, | 0.3% |
| vitamin E, vitamin C palmitate | |

### Example 17

### Silicone in Water Emulsion

A silicone in water emulsion is prepared with the following ingredients at the listed percentages:

| Phase | Trade Name | INCI | % w/w |
|---|---|---|---|
| A | Phosal 50PG | Lecithin, | 0-6.00 |
| | | Propylene glycol | |
| | Arlasilk Phospholipid | Linoleamidopropyl | 0-5.00 |
| | EFA | PG-Dimonium Chloride | |
| | | Phosphate | |
| | Arlasilk Phospholipid | Linoleamidopropyl | 0-5.00 |
| | PLN | PG-Dimonium Chloride | |
| | | Phosphate Dimethicone | |
| | PEG-4 | PEG-4 | 0-10.00 |
| | MMP Inhibitor | | |
| | 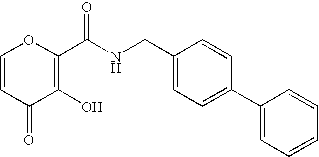 | | |
| | BHT | BHT | 0.20 |
| | Vitamin C | Ascorbic Acid | 0.20 |
| | Vitamin E | Tocopherol | 0.20 |
| B | Glycerin | Glycerin | 3.00-6.00 |
| | Water | Water | q.s. |
| C | DC RM2051 | Sodium Polyacrylate | 2.00-7.00 |
| | | (and) Dimethicone (and) | |
| | | Cyclopentasiloxane (and) | |
| | | Trideceth-6 (and) | |
| | | PEG/PPG-18/18 Dimethicone | |
| | DC 556 | Phenyl Trimethicone | 2.00-6.00 |
| | DC FZ-3196 | Caprylyl Methicone | 2.00-5.00 |
| | Squalane | Squalane | 2.00-3.00 |
| | DC 9045 | Cyclopentasiloxane, | 5.00-20.00 |
| | | Dimethicone | |
| | | crosspolymer | |
| D | DC 7-3101 | Cyclopentasioxane (and) | 5.00-10.00 |
| | | Dimethicone | |
| | | Crosspolymer (and) | |
| | | Dimethicone (and) | |
| | | Laureth-23 (and) Laureth-4 | |
| E | Optiphen plus | Phenoxyethanol (and) | 0.50-1.00 |
| | | Caprylyl Glycol (and) | |
| | | Sorbic Acid | |

The MMP inhibitor, BHT, vitamin C, and vitamin E are added to the remaining ingredients in Phase A, then heated to 70° C. and mixed until homogeneously dispersed. Phase C, composed of glycerin and water, is also heated to 70° C. Phase A is slowly added to Phase B under high shear, forming Phase AB, a phospholipid mixture. Phase AB is then allowed to cool to 30° C. while mixing. The ingredients in Phase C are mixed at room temperature. Phase AB is slowly added to Phase C under high shear. Then Phases D and E are added sequentially and mixed until uniform to complete the formulation of the lotion.

### Example 18

### Water in Silicone Emulsion

A water in silicone emulsion is prepared with the following ingredients at the listed percentages:

| Phase | Trade Name | INCI | % w/w |
|---|---|---|---|
| A | Phospholipon 90 G | Lecithin | 0-3.10 |
| | PEG-4 | PEG-4 | 5.00-15.00 |
| | Arlasilk Phospholipid | Linoleamidopropyl | 0-5.00 |
| | PLN | PG-Dimonium | |
| | | Chloride Phosphate | |
| | | Dimethicone | |
| | Cetiol HE | PEG-7 Glyceryl Cocoate | 0-10.00 |
| | MMP Inhibitor | | |
| | 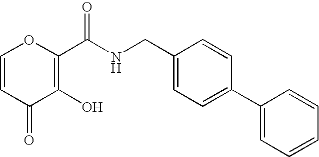 | | 0.01-0.50 |
| | BHT | BHT | 0.20 |
| | Vitamin C Palmitate | Ascorbyl Palmitate | 0.20 |
| | Vitamin E | Tocopherol | 0.20 |
| B | Glycerin | Glycerin | 3.00-8.00 |
| | Lipocare HA/EC | Hyaluronic Acid | 0-1.00 |
| | | (and) Echinacin | |
| | Water | Water | q.s. |
| | Sodium chloride | Sodium chloride | 0.75-2.00 |
| C | Gransil WO | Cyclopentasiloxane (and) | |
| | | Polysilicone- 11 | 20.00-35.00 |
| | | (and) Glyceryl Laurate | |
| | | (and) Cetyl | |
| | | PEG/PPG 10/1 Dimethicone | |
| | | (and) PEG/PPG- | |
| | | 18/18 Dimethicone | |
| | Gransil RPS | Cyclopentasiloxane | 0-5.00 |
| | | (and) Polysilicone-11 | |
| | DC 200 0.65cst | Cyclomethicone | 0-5.00 |
| | DC 9701 | Dimethicone/Vinyl | 0-4.00 |
| | | Dimethicone | |
| | | Crosspolymer | |
| | | (and) Silica | |
| | Microsilk 920 | Polypropylene/PTFE | 0-1.00 |
| | Abil EM-90 | Cetyl PEG/PPG-10/1 | 0-1.00 |
| | | Dimethicone | |
| | DC 5225C | Cyclopentasiloxane (and) | 0-2.00 |
| | | PEG/PPG-18/18 | |
| | | Dimethicone | |
| | Crodamol PMP | PPG-2 Myristyl | 0-2.00 |
| | | Ether Propionate | |
| | Shea butter | Butyrospermum Parkii | 0-2.00 |
| | | (Shea Butter) Fruit | |
| D | Optiphen plus | Phenoxyethanol (and) | 0.50-1.00 |
| | | Caprylyl Glycol | |
| | | (and) Sorbic Acid | |

The MMP inhibitor, BHT, vitamin C, and vitamin E are added to the remaining ingredients in Phase A, then heated to 70° C. and mixed until homogeneously dispersed. Phase B is also mixed and heated to 70° C. Under high shear, Phase A is added to Phase B until uniform. Phase AB is allowed to cool to 30° C. while mixing. Phase C is mixed until uniform at room temperature. Phase AB is added to Phase C until a uniform emulsion is achieved. Finally, Phase D is added and mixed until uniform to complete the formulation of the lotion.

### Example 19

### Glycol in Silicone Emulsion

A glycol in silicone emulsion is prepared with the following ingredients at the listed percentages:

| Phase | Trade Name | INCI | % w/w |
|---|---|---|---|
| A | Tospearl 145A | Polymethylsilsesquionane | 0-2.50 |
| | DC Aerogel 2270 | Silica Silylate | 0-0.20 |
| | DC 9045 | Cyclopentasiloxane, Dimethicone | q.s. |
| | | crosspolymer | |
| | DC 200 0.65 cst | Dimethicone | 0-13.60 |
| | DC 245 | Cyclopentasiloxane | 0-24.00 |
| | Chemsil K12 | Dimethicone (and) Dimethicone PEG-10/15 | 0-14.00 |
| | | Crosspolymer | |
| | Chemsil K51 | Cyclopentasiloxane (and) | 0-54.00 |
| | | Dimethicone/Vinyl Dimethicone | |
| | | Crosspolymer) | |
| | Talc N-12 | Talc, Isopropyl titanium tricostearate | 0-5.20 |
| | Dryflo AF | Corn Starch Modified | 0-5.70 |
| | Microsilk 419 | Polyethylene/PTFE | 0-1.70 |
| B | PEG-4 | PEG-4 | 10.00-20.00 |
| | 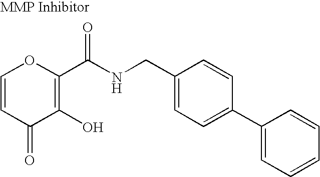 | | 0.01-0.50 |
| | BHT | BHT | 0.20 |
| | Vitamin C Palmitate | Ascorbyl Palmitate | 0.20 |
| | Vitamin E Acetate | Tocopheryl Acetate | 0.20 |
| C | Glycerin | Glycerin | 0-6.00 |
| D | SF1540 | Cyclopentasiloxane, PEG/PPG-20/15 | 0-3.00 |
| | | dimethicone | |

If included, TOSPEARL 145A and AEROGEL 2270 are mixed in DC 9045, DC 200 0.65 cst, and DC 245 before adding the remaining ingredients in Phase A. Phase B ingredients are heated to 70° C. and mixed until homogeneously dispersed, then cooled to 30° C. while mixing. Glycerin is added to Phase B to form Phase BC. Phase BC is then added to Phase A and mixed until uniform. SF 1540 is added and mixed until uniform to complete the formulation of the cream.

### Example 20

### Liquid Crystal Gel Network Emulsion

A liquid crystal gel network cream is prepared with the following ingredients at the listed percentages:

| Phase | Trade Name | INCI | % w/w |
|---|---|---|---|
| A | Polyglycol 200 | PEG-4 | 5.00-20.00 |
| | Atlas G2330 | Polyoxyethylene-30 Sorbitol | 0-10.00 |
| | Intermediate | | |
| | DMI | Dimethyl Isosorbide | 0-5.00 |
| | Ethoxydiglycol | Ethoxydiglycol | 0-5.00 |
| | 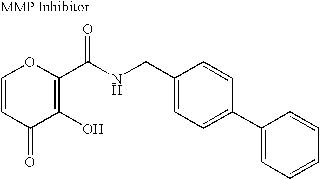 | | 0.01-0.50 |
| | BHT | BHT | 0.20 |
| | Vitamin C | Ascorbic Acid | 0.20 |
| | Vitamin E | Tocopheryl Acetate | 0.20 |
| B | Biobase EP | Sodium Lauroyl Lactylate, Lecithin, Cetearyl | 5.00-10.00 |
| | | Alcohol Glyceryl Stearate | |
| | Acconon CC-6 | PEG-6 Caprylic/Capric Glycerides | 0-1.00 |
| | Tween 20 | Polysorbate 20 | 0-1.00 |
| | Captex 300 | Caprylic Capric Triglyceride | 0-2.00 |
| C | Water | Water | q.s. |
| | Carbopol ETD | Acrylates/C10-30 alkyl acrylate crosspolymer | 0.15-0.40 |
| | 2020 | | |
| D | AMP | Aminomethyl Propanol | 0.10-0.30 |
| E | Germazide PMP | Phenoxyethanol, Chlorphenesin, | 0.50-1.00 |
| | | Methylparaben, Propylparaben | |

The MMP inhibitor, BHT, vitamin C, and vitamin E are mixed with PEG-4 at 70° C. until homogeneously dispersed to form Phase A. Phase B is made by mixing BIOBASE EP, ACCONON CC-6, TWEEN 20, and CAPTEX 300, and is heated to 70° C. Phase A is added to Phase B to form Phase AB. Phase C is made by dispersing CARBOPOL ETD2020 in water, then heated to 70° C. Phase AB is added to Phase C. The resulting mixture is allowed to cool to 40° C. while mixing, then aminomethyl propanol is added to neutralize. Finally, OPTIPHEN PLUS is added and mixed until uniform to complete the formulation of the lotion.

### Example 21

### Nanoemulsion

A submicron/nanoemulsion cream is prepared with the following ingredients at the listed percentages:

| Phase | Trade Name | INCI | % w/w |
|---|---|---|---|
| A | Nanogel CCT | Capric/Caprylic Trigylceride, Water, | 5.00-10.00 |
| | | Glycerin, Laureth-23, Sodium | |
| | | Dicocoylethylenediamine PEG-15 Sulfate, | |
| | | Sodium Lauroyl Lactylate, Behenyl Alcohol, | |
| | | Glyceryl Stearate Citrate | |
| | Cetiol HE | PEG-7 Glyceryl Cocoate | 4.00-9.00 |
| B | Polyglycol 200 | PEG-4 | 10.00-20.0 |
| | 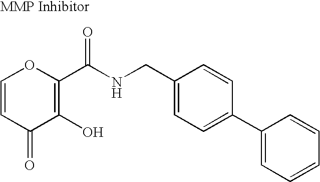 | | 0.01-0.50 |
| | BHT | BHT | 0.20 |
| | Vitamin C | Ascorbic Acid | 0.20 |
| | Vitamin E | Tocopheryl Acetate | 0.20 |
| C | Water | Water | qs |
| | Ultrez 10 | Carbomer | 0.30-0.80 |
| D | AMP | Aminomethyl Propanol | 0.20-0.70 |
| | Germazide PMP | Phenoxyethanol, Chlorphenesin, | 0.50 |
| | | Methylparaben, Propylparaben | |

NANOGEL CCT and CETIOL HE are mixed together until uniform to create Phase A. The MMP inhibitor, BHT, vitamin C, and vitamin E are mixed with PEG-4 at 70° C. until homogeneously dispersed to form Phase B. After heating Phase A to 70° C., Phase B is added to it until uniform. Phase C is made by dispersing ULTREZ 10 in water, then added to Phase AB. Finally, aminomethyl propanol and OPTIPHEN PLUS are added sequentially and mixed until uniform to complete the formulation of the lotion.

### Example 22

### Phospholipid/Liposomal Emulsion

A phospholipid emulsion which spontaneously forms liposomal vesicles is prepared with the following ingredients at the listed percentages:

| Phase | Trade Name | INCI | % w/w |
|---|---|---|---|
| A | Phosal 50PG | Lecithin, Propylene glycol | 5.00-12.00 |
| | 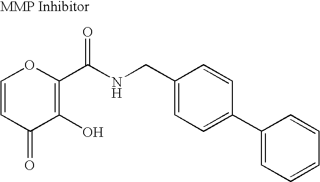 | | 0.01-0.50 |
| | BHT | BHT | 0.20 |
| | Vitamin C | Ascorbic Acid | 0.20 |
| | Vitamin E Acetate | Tocopheryl Acetate | 0.20 |
| B | Water | Water | qs |
| C | Sepiplus 400 | Polyacrylate, Polyisobutene, Polysorbate 20 | 1.50-3.00 |
| D | Optiphen plus | Phenoxyethanol (and) Caprylyl Glycol (and) | 0.50-1.00 |
| | | Sorbic Acid | |

The MMP inhibitor, BHT, vitamin C, and vitamin E are mixed with PHOSAL 50PG at 70° C. until homogenously dispersed. This dispersion is slowly added to water under high shear. Finally, SEPIPLUS 400 and OPTIPHEN PLUS are added sequentially and mixed until uniform to complete the formulation of the lotion.

### Example 23

### PEG/Water Serum

A polyethylene glycol and water serum is prepared with the following ingredients at the listed percentages:

| Phase | Trade Name | INCI | % w/w |
|---|---|---|---|
| A | Polyglycol 200 | PEG-4 | 70.00 |
| | Atlas G2330 | Polyoxyethylene-30 Sorbitol | 1.00 |
| | Intermediate | | |
| | 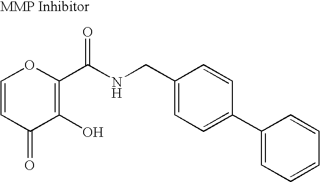 | | 0.01-0.50 |
| | BHT | BHT | 0.20 |
| | Vitamin C | Ascorbic Acid | 0.20 |
| | Vitamin E | Tocopherol | 0.20 |
| B | Water | Water | 27.90 |
| | Carbopol Ultrez 20 | Acrylates/C10-30 Alkyl Acrylate | 0.10 |
| | | Crosspolymer | |
| C | TEA | Triethanolamine | 0.10 |
| | Optiphen plus | Phenoxyethanol (and) Caprylyl | 0.50 |
| | | Glycol (and) Sorbic Acid | |

The MMP inhibitor, BHT, vitamin C, and vitamin E are fully solubilized in PEG-4 by heating to 70° C. and mixing until clear. Then Phase A is cooled to 30° C. Separately, CARBOPOL ULTREZ 20 is dispersed in water at room temperature to form Phase B. Phase A is mixed with Phase B until homogeneous. Triethanolamine and OPTIPHEN PLUS are added sequentially and mixed until uniform to complete the serum.

### Example 24

### Cationic Emulsion

A cationic emulsion cosmetic cream is prepared with the following ingredients at the listed percentages:

| Phase | Trade Name | INCI | % w/w |
|---|---|---|---|
| A | Arlasilk Phospholipid | Sodium Borageamidopropyl PG-Dimonium | 0-3.00 |
| | GLA | Chloride Phosphate | |
| | Arlasilk Phospholipid | Linoleamidopropyl PG-Dimonium Chloride | 0-4.00 |
| | PLN | Phosphate Dimethicone | |
| | GMS | Glyceryl Monostearate | 1.00-1.50 |
| | Crodamol PMP | PPG-2 Myristyl Ether Propionate | 2.00-7.00 |
| | Borage Oil | | 1.00-5.00 |
| | | Cetyl Alcohol | 0.50-1.00 |
| | Ganex V-220 | VP/Eicosene Copolymer | 0-2.00 |
| B | PEG-4 | PEG-4 | 5.00-15.00 |
| | 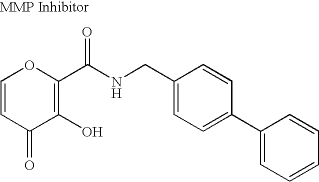 | | 0.01-0.50 |
| | BHT | BHT | 0.20 |
| | Vitamin C | Ascorbic Acid | 0.20 |
| | Vitamin E Acetate | Tocopheryl Acetate | 0.20 |
| C | Water | Water | qs |
| | Glycerin | Glycerin | 3.00-8.00 |
| D | Thickener | | 0.70-4.00 |
| E | Jeesperse HD | Isododecane, dimethicone crosspolmyer-3, | 0-5.00 |
| | | POE-4 lauryl ether, POE-10 cetyl ether | |
| F | Preservative | | 0.70 |

All ingredients in Phase A are mixed together. The MMP inhibitor, BHT, vitamin C, and vitamin E are mixed with PEG-4 at 70° C. until homogeneously dispersed. Phase C is added to Phase B, maintaining the temperature at 70° C. Phase A is heated to 70° C., and mixed with Phase BC under high shear. The resulting mixture is cooled to 30° C. while mixing. Then, Phase D, E and F are added sequentially until uniform to complete the formulation.

### Example 25

### Solid Cosmetic Stick

A solid non greasy cosmetic stick that improves ease of application is prepared with the following ingredients at the listed percentages:

| Phase | Trade Name | INCI | % w/w |
|---|---|---|---|
| A | Carnauba Wax | Copernicia Cerifera | 0.00-2.00 |
| | | (Carnauba) Wax | |
| | Ceresine Wax | Ceresin | 0.00-8.00 |
| | Candelila Wax | Euphorboria Cerifera | 3.00-8.00 |
| | | (Candelilla) Wax | |
| | Microcrystalline | Microcrystalline Wax | 0.00-2.00 |
| | Wax | | |
| | GE-IS | Isostearyl Glyceryl Ether | 64.60 |
| | Jojoba Oil | Simmondsia Chinensis | 0.00-10.00 |
| | | (Jojoba) Seed Oil | |
| | Ozokerite Wax | Ozokerite | 0.00-1.00 |
| | Beeswax, White | Beeswax | 0.00-12.00 |
| | Castor Oil | Ricinus Communis (Castor) | 0.00-37.00 |
| | | Seed Oil | |
| | AC Milk Lipids | Milk Lipids | 0.00-29.60 |
| | Water | Water | 0.00-20.95 |
| | Propylene Glycol | Propylene Glycol | 0.00-66.70 |
| B | Lanette 18 | Stearyl Alcohol | 0.00-0.20 |
| | Unichem SS | Sodium Stearate | 0.00-0.50 |
| | Salt | Sodium Chloride | 0.00-0.50 |
| C | 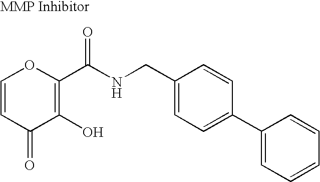 | | 0.01-0.50 |
| | Lipo Polyglycol | PEG-4 | 0.00-10.00 |
| | 200 | | |
| | Vitamin E | Tocopheryl Acetate | 0.20 |
| | Acetate | | |
| | Brij 30 | Laureth-4 | 0.00-10.00 |
| D | Optiphen Plus | Phenoxyethanol (and) | 0.00-0.75 |
| | | Caprylyl Glycol (and) Sorbic | |
| | | Acid | |
| | Total | | 100.00 |

The MMP inhibitor and vitamin E are dispersed in PEG-4 or Laureth-4 by heating to 70° C. and mixing until uniform to create Phase C. Phase A is made heating ingredients to 85° C. Phase B ingredients are then added to Phase A. The resulting mixture is cooled to 70° C., and Phase C and Optiphen Plus are added. The resulting mixture is allowed to cool to 50° C. and then hot filled into a final package.

### Example 26

### Powder

A free flowing powder is prepared with the following ingredients at the listed percentages:

| Phase | Trade Name | INCI | % w/w |
|---|---|---|---|
| A | Polyglycol 200 | PEG-4 | 55.00 |
| | Atlas G2330 | Polyoxyethylene-30 Sorbitol | 1.00 |
| | Intermediate | | |
| | Shea Butter | | 5.00 |
| | 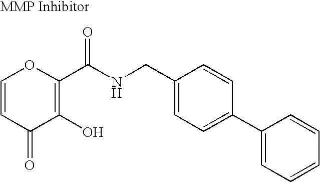 | | 0.15 |
| | BHT | BHT | 0.20 |
| | Vitamin C | Ascorbic Acid | 0.20 |
| | Vitamin E Acetate | Tocopheryl Acetate | 0.20 |
| B | Microsponge HSB | Amcol International Corp | qs |
| | Polymer Powder or | | |
| | Poly-pore | | |

Phase A is blended to solubilize the MMP Inhibitor and heated to 70° C. to dissolve the shea butter. Phase A is blended with Phase B to adsorb liquid into the porous polymer matrix. Specific details are further outlined in the review article entitled “Microsponge Delivery System” Chadawar & Shaji, Current Drug Delivery, 2007, Vol. 4, No. 2, pp. 123-129; incorporated herein by reference.

### Example 27

### Cream

A rich moisturizing cream is prepared with the following ingredients at the listed percentages:

| Phase | Trade Name | INCI | % w/w |
|---|---|---|---|
| A | Water | Water | 56.25 |
| | Ultrez 10 | Carbomer | 0.25 |
| | Pemulen TR-2 | Acrylates/C10-30 | 0.25 |
| | | alkylacrylate crosspolymer | |
| | EDTA | EDTA | 0.10 |
| | Glycerin | Glycerin | 0.50 |
| B | Vitamin E | Tocopherol | 0.10 |
| | Vitamin C | Ascorbic acid | 0.10 |
| | BHT | BHT | 0.10 |
| | 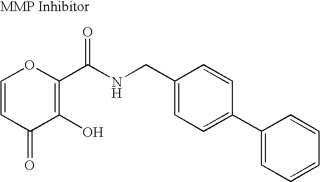 | | 0.20 |
| | Arlasolve DMI- | Dimethyl isosorbide | 24.50 |
| | PC | | |
| | Cetiol HE | PEG-7 glyceryl cocoate | 5.00 |
| | Arlacel 80 | Sorbitan oleate | 0.15 |
| | Liponate GC | Caprylic/capric triglycerides | 7.50 |
| C | Sodium | Sodium hydroxide | 1.00 |
| | hydroxide, 20% | | |
| D | Germazide PMP | Phenoxyethanol, | 1.00 |
| | | chlorophenesin, methyl | |
| | | paraben, propyl paraben | |
| E | Rheosol AVH | Sodium polyacrylate, | 3.00 |
| | | ethylhexyl stearate, | |
| | | trideceth-6 | |

ULTREZ 10 and PEMULEN TR-2 are dispersed in water, then heated to 70° C. Then EDTA and GLYCERIN are added to complete Phase A.

Separately, the MMP inhibitor, vitamin C, vitamin E, and BHT are dissolved in ARLASOLVE DMI-PC by heating to 60° C. CETIOL HE, ARLACEL 80, and LIPONATE GC are added to form Phase B, which is then heated to 70° C. Phase A and Phase B are mixed together at 70° C., then cooled to 60° C. Sodium hydroxide is added to neutralize the formulation, and mixed until cooled to 40° C. GERMAZIDE PMP is added, and the composition is mixed until cooled to room temperature. Finally, RHEOSOL AVH is added and mixed well to complete the formulation of the cream.

### Example 28

### Anhydrous Serum

An effective high-delivery serum is prepared with the following ingredients at the listed percentages:

| Phase | Trade Name | INCI | % w/w |
|---|---|---|---|
| A | DC 9045 | Cyclopentasiloxane, | 62.00 |
| | | Dimethicone crosspolymer | |
| | DC 245 | Cyclopentasiloxane | 16.35 |
| | Tospearl 145A | | 2.50 |
| B | PEG-4 | PEG-4 | 9.60 |
| | 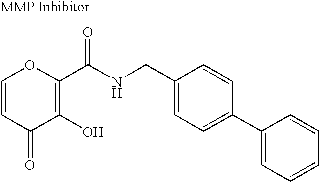 | | 0.20 |
| | BHT | BHT | 0.20 |
| | Vitamin C | Ascorbyl Palmitate | 0.20 |
| | Palmitate | | |
| | Vitamin E | Tocopheryl Acetate | 0.20 |
| | Acetate | | |
| | Glycerin | Glycerin | 6.00 |
| C | SF 1540 | Cyclopentasiloxane, | 2.75 |
| | | PLG/PPG-20/15 dimethicone | |

DC 9045, DC 245, and TOSPEARL 145A are mixed together until uniform at room temperature to form Phase A. PEG-4, the MMP inhibitor, BHT, vitamin C, and vitamin E are mixed together and heated to 70° C. to form a homogenous dispersion, then cooled to 25° C. Glycerin is then added to complete Phase B. Phase B is added to Phase A, and mixed for 10 minutes. Phase C is then added slowly and mixed until uniform to complete the serum.

### Example 29

### Water-in-Silicone Lotion

A light, moisturizing water-in-silicone lotion is prepared with the following ingredients at the listed percentages:

| Phase | Trade Name | INCI | % w/w |
|---|---|---|---|
| A | Gransil WO | Cyclopentasiloxane (and) Polysilicone-11 | 35.00 |
| | | (and) Glyceryl Laurate (and) Cetyl PEG/PPG | |
| | | 10/1 Dimethicone (and) PEG/PPG- 18/18 | |
| | | Dimethicone | |
| | Gransil RPS | Cyclopentasiloxane (and) Polysilicone-11 | 5.00 |
| | DC 9701 | Dimethicone/Vinyl Dimethicone | 4.00 |
| | | Crosspolymer (and) Silica | |
| | DC 200 0.65 cst | Cyclomethicone | 4.50 |
| B | Cetiol HE | PEG-7 Glyceryl Cocoate | 10.00 |
| | PEG-4 | PEG-4 | 5.00 |
| | 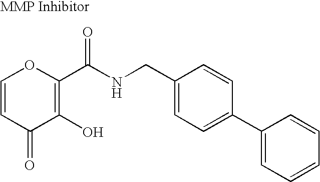 | | 0.20 |
| | BHT | BHT | 0.20 |
| | Vitamin C Palmitate | Ascorbyl Palmitate | 0.20 |
| | Vitamin E Acetate | Tocopheryl Acetate | 0.20 |
| C | Water | Water | 29.70 |
| | Glycerin | Glycerin | 3.00 |
| | Sodium chloride | Sodium chloride | 1.50 |
| | Lipocare HA/EC | Hyaluronic Acid and Echinacin | 1.00 |
| D | Optiphen plus | Phenoxyethanol (and) Caprylyl Glycol (and) | 0.50 |
| | | Sorbic Acid | |

All components of Phase A are mixed together until uniform.

Separately, all components of Phase B are mixed at 70° C. until a uniform dispersion is formed. All components of Phase C are also mixed and heated to 70° C., then added to Phase B. Phase BC is then mixed while being cooled to 25° C. After cooling, it is mixed with Phase A until uniform. Finally, OPTIPHEN PLUS is added to complete the formulation of the lotion.

### Example 30

### Face Mask

An oil-in-water face mask is prepared with the following ingredients at the listed percentages:

| Phase | Trade Name | INCI | % w/w |
|---|---|---|---|
| A | Emulgade PL | Cetearyl Glucoside (and) | 4.00 |
| | 68/50 | Cetearyl Alcohol | |
| | Eumulgin SG | Sodium Stearoyl Glutamate | 1.00 |
| | Cegesoft PS 6 | Vegetable Oil | 5.00 |
| | Cosmedia SP | Sodium Polyacrylate | 1.00 |
| B | Vitamin E | Tocopherol | 0.10 |
| | Vitamin C | Ascorbic acid | 0.10 |
| | BHT | BHT | 0.10 |
| | 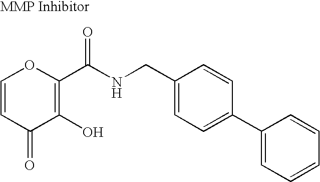 | | 0.20 |
| | Cetiol HE | PEG-7 glyceryl cocoate | 12.00-20.00 |
| | PEG-4 | PEG-4 | 6.00-10.00 |
| | Arlasilk | Linoleamidopropyl PG- | 6.00-10.00 |
| | Phospholipid | Dimonium Chloride | |
| | EFA | Phosphate | |
| | Phosphate- | Water, Sodium chloride, | 9.00 |
| | buffered saline, | Potassium chloride, | |
| | 10X concentrate | Disodium phosphate, | |
| | | Potassium phosphate | |
| C | Water | Water | q.s. |
| | Glycerin | Glycerin | 5.00-10.00 |
| | Keltrol CGT | Xantham Gum | 0.20 |
| D | Optiphen Plus | Phenoxyethanol, Caprylyl | 0.75 |
| | | Glycol, Sorbic Acid | |

All ingredients of Phase A are mixed together and heated to 70° C.

The ingredients of Phase B are mixed together at 70° C. until fully solubilized. Phase C is mixed and heated to 70° C., then added to Phase B to create a homogeneous solution. Phase A is added slowly to Phase BC at 70° C. The mixture is allowed to cool to 35° C. while mixing, and then OPTIPHEN PLUS is added. Finally, the finished formulation is stirred until it is cooled to room temperature.

### Example 31

### Water-in-Silicone Cream

A light water-in-silicone cream is prepared with the following ingredients at the listed percentages:

| Phase | Trade Name | INCI | % w/w |
|---|---|---|---|
| A | DC 5225C | Cyclopentasiloxane, | 10.00 |
| | | PEG/PPG-18/18 dimethicone | |
| | DC 245 | Cyclomethicone | 7.50 |
| | DC 556 | Phenyl trimethicone | 7.50 |
| | DC 9701 | Dimethicone/Vinyl | 4.00 |
| | | Dimethicone Crosspolymer, | |
| | | Silica | |
| B | Vitamin E | Tocopherol | 0.10 |
| | Vitamin C | Ascorbic acid | 0.10 |
| | BHT | BHT | 0.10 |
| | 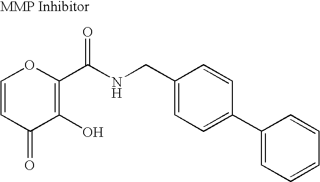 | | 0.20 |
| | Cetiol HE | PEG-7 glyceryl cocoate | 12.00 |
| | PEG-4 | PEG-4 | 6.00 |
| | Arlasilk | Linoleamidopropyl PG- | 6.00 |
| | Phospholipid | Dimonium Chloride | |
| | EFA | Phosphate | |
| | Phosphate- | Water, Sodium chloride, | 6.00 |
| | buffered saline, | Potassium chloride, | |
| | 10X concentrate | Disodium phosphate, | |
| | | Potassium phosphate | |
| C | Water | Water | 40.00 |
| D | Optiphen Plus | Phenoxyethanol, Caprylyl | 0.50 |
| | | Glycol, Sorbic Acid | |

All ingredients in phase A are mixed together.

Separately, all ingredients in Phase B are mixed at 70° C. until fully solubilized. Then, Phase C at room temperature is mixed into Phase A until the mixture cools to 30° C. Phase BC is then added very slowly to Phase A under high shear until a homogeneous emulsion is made. After mixing under high shear for ten minutes, Phase D is added to complete the formulation of the cream.

### Example 32

### Evaluation of Cosmetic Compositions

Photoaging of the skin typically manifests itself as worsening appearance of the skin, including an increased appearance of mottled hyperpigmentation, wrinkles, coarseness of skin, redness, and pores. In this study, the MMP inhibitor,

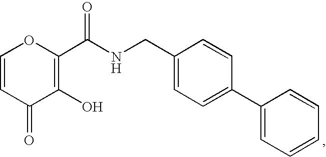

was evaluated in female subjects 40-65 years of age with mild to moderate skin aging to determine its benefit on the appearance of aging.

The study was designed as a double-blind, placebo-controlled, randomized 16-week clinical study to evaluate the benefit of the MMP inhibitor,

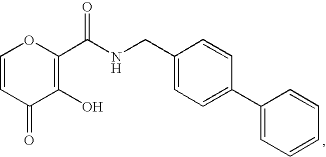

in reducing the appearance of aging. Subjects enrolled in the study were between 40-65 years of age with mild to moderate skin age scores. A randomization table was used to determine which subjects would receive the study cream and which would receive the placebo cream. Subjects were instructed to apply the assigned cream to their face twice a day (once in the morning and once in the evening) after their face was washed and dried. A pea-sized drop of the test article was applied to each quadrant of the face to form a thin layer over the entire face. Follow-up visits were scheduled at 2, 4, 8, 12, and 16 weeks after the cream application was started.

Subjects were instructed to apply the test or placebo cream to the face twice daily (once in the morning and once in the evening) over the entire 16 week study period. Each subject was instructed to wash and dry her face before applying the test cream. A pea-size drop of the test article was applied to each quadrant of the face to form a thin layer over the entire face. The formulation of the test article with the MMP inhibitor,

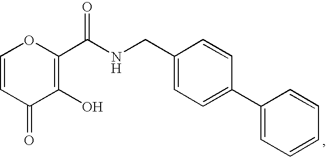

used in this study is described in Example 27.

The effect of the MMP inhibitor on the subject's skin was assessed using the Physician Global Assessment of facial skin appearance and the assessment of the following individual skin appearance parameters on a scale from 0 to 9: fine wrinkling, mottled hyperpigmentation, age spots, tactile roughness, coarse wrinkling, telangiectasia, pore size, sallow tone, and sag. The cosmetic appearance of telangiectasia may be perceived as increased facial redness. Other criteria were also used including profilometry of crow's feet region using silicone replicas.

### Evaluation

At the screening visit, subjects had their Fitzpatrick Skin Type assessed according to the table below. Only subjects with a Fitzpatrick Skin Type of I-III were enrolled in the study. (Fitzpatrick, Arch. Dermatol. 124:869-871, 1988; incorporated herein by reference).

| Fitzpatrick | | |
|---|---|---|
| Skin Type | Skin Color | Characteristics |
| I | White; very fair; red or blond hair; | Always burns, never tans |
| | blue eyes; freckles | |
| II | White; fair; red or blond hair; blue, | Usually burns, tans with |
| | hazel or green eyes | difficulty |
| III | Cream white; fair with any eye or | Sometimes mild burn, |
| | hair color; very common | gradually tans |
| IV | Brown; typical Mediterranean | Rarely burns, tans with |
| | Caucasian skin | ease |
| V | Dark Brown; mid-eastern skin | Very rarely burns, tans |
| | types | very easily |
| VI | Black | Never burns, tans very |
| | | easily |

Subjects also had their level of photodamage assessed at the screening visit using the Glogau Skin Age Classification Scale as described in the table below. (Glogau, Semin. Cutan. Med. Surg. 15(3):134-8, 1996; incorporated herein by reference). Only subjects with a Glogau Skin Age of Type II or III were enrolled in the study.

| Glogau | | |
|---|---|---|
| Photodamage | | |
| Classification | Typical Attributes | Typical Treatments |
| Type I | Minimal to no | Daily skin protection with a sunscreen |
| No Wrinkles | discoloration or wrinkling | Moisturizers and/or cosmetic lotions |
| | No keratoses (skin over- | containing alpha |
| | growths) | hydroxy and antioxidants |
| | Generally no need for | |
| | foundation or makeup | |
| Type II | Wrinkling as skin moves | Daily skin protection with a sunscreen |
| Wrinkles in | Slight lines near the eyes | A cosmetic lotion containing alpha |
| motion | and mouth | hydroxy acid and antioxidants or retinol |
| | Usually a need for some | Prescription medicines containing |
| | foundation | tretinoin |
| | No visible keratoses | Prescription medicines containing |
| | | hydroquinone |
| Type III | Visible wrinkles all the | Daily skin protection with a sunscreen |
| Wrinkles at | time | A cosmetic lotion containing alpha |
| rest | Noticeable discolorations | hydroxy acid and |
| | Visible keratoses | antioxidants or retinol |
| | Generally a need for | Prescription medicines containing |
| | heavy foundation | tretinoin or hydroquinone |
| | | Light chemical peels |
| | | Light laser resurfacing |
| Type IV | Wrinkles throughout | Daily skin protection with a sunscreen |
| Only Wrinkles | Yellow or gray color to | Deep, stronger chemical peels |
| | skin | Deep dermabrasion |
| | Prior skin cancer | Deep laser resurfacing |
| | Makeup not usable | Soft tissue augmentation (injections of |
| | because it cakes and | collagen or fat transfer) |
| | cracks | |

A Physician Global Assessment of facial skin appearance using the Griffiths scale (0-8) was performed at all study visits. (Griffiths et al., Arch. Dermatol. Res. 128:347-51, 1992; incorporated herein by reference). An assessment of individual skin parameters including fine wrinkling, mottled hyperpigmentation, age spots, tactile roughness, coarse wrinkling, redness, pore size, sollowness, and sag was scored on a scale from 0-9 at all study visits based on the following table:

| | Severity of skin |
|---|---|
| Score | parameter appearance |
| 0 | No appearance |
| 1-3 | Mild |
| 4-6 | Moderate |
| 7-9 | Severe |

Silicone replicas of the crow's feet area (“profilometry”) were made at day 0, week 8, and week 16.

### Statistical Analysis

Analyses of the data were conducted utilizing nonparametric Mann-Whitney test of significance in addition to a General Linear Model, PROC GLM (SAS, Cary, N.C.), treating time as continuous factor. For the Mann-Whitney test, the change in the dermatologist assessed scores at the 16 week timepoint from the scores of each individual at baseline for the treatment group were compared against the changes observed for the placebo group. The probability that the differences observed between treatment groups resulted purely by chance is given by the p-value. The PROC GLM method is a statistical test that collects the change in the dermatologist assessment score for each subject at each timepoint, relative to the baseline score, to determine a trend in the change of the scores as a result of time and treatment effects. The model considered the effect of time independently as well as the interaction of time with the treatment on the change from the baseline score. Like the indicator of statistical significance for the Mann-Whitney test, the p-value is reported as an output of this statistical test. The p-value provides a measure of confidence in the trends generated by the data and indicates the likelihood that a score change resulted randomly. For example, a p-value of 0.05 indicates a 5% probability that the outcome resulted from chance alone. In this study, p-values less than 0.05 were, by convention, regarded as statistically significant. Statistically significant outcomes are not likely to have occurred by chance.

For the silicone replicas, the data used in the statistical analyses was the change from baseline. Within-treatment analysis of the skin replica parameters changes from baseline were evaluated using Student's t-test for paired data. Between-treatment analyses were conducted utilizing analysis of covariance with the baseline value as the covariate.

### Results

The baseline clinical characteristics for the subjects enrolled in the study are summarized in the table below. No significant differences were detected between the two groups for each of the appearance attributes at the beginning of the study.

| Characteristic | Group | 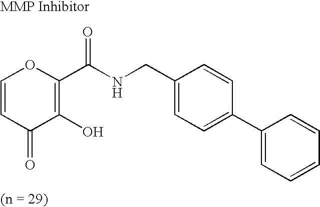 | Control(vehicle only)(n = 29) |
|---|---|---|---|
| Susceptibility to sunburn (Fitzpatrick) | I | 3 (10%) | 4 (14%) |
| | II | 15 (52%) | 15 (52%) |
| | III | 11 (38%) | 10 (34%) |
| Skin age (Glogau) | II | 6 (21%) | 13 (45%) |
| | III | 23 (79%) | 16 (55%) |
| Age: mean (median) | | 52.0 (54) | 54.4 (53) |
| Griffiths score: mean (median) | | 3.9 (3) | 3.6 (3) |
| Overall Formulation Use/Day | | 0.99 (1.02) | 1.09 (1.05) |
| Mean (median): g/day | | | |

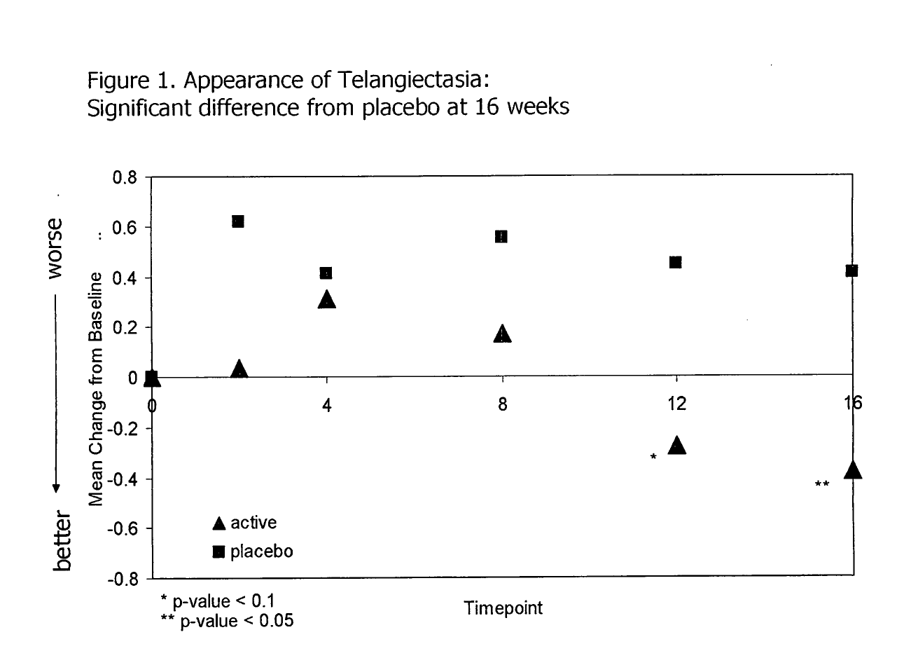

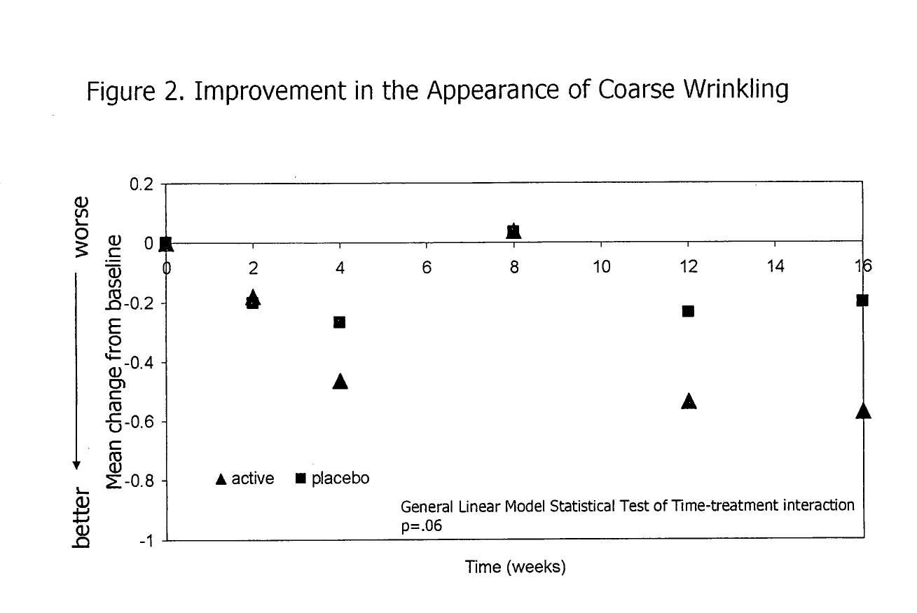

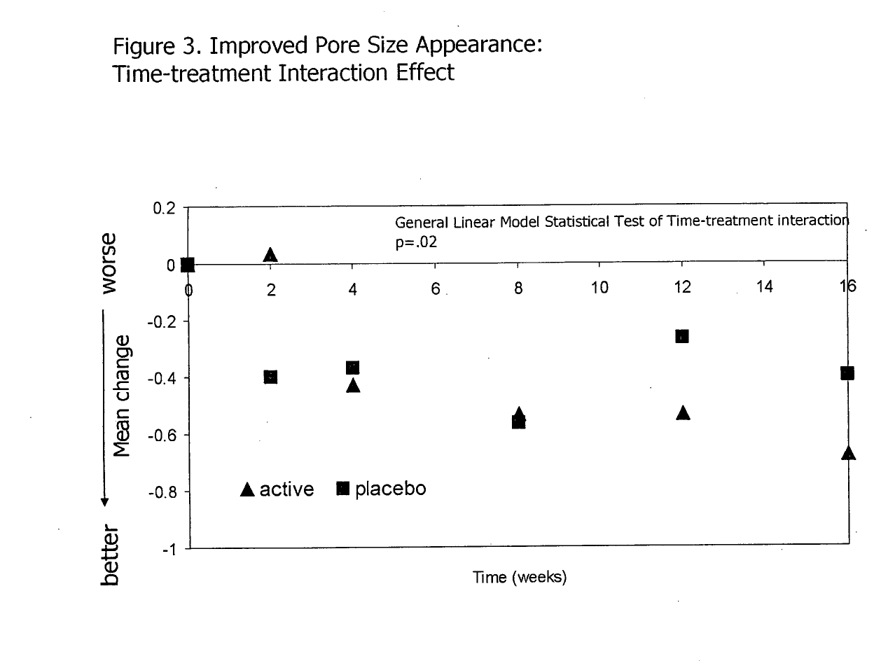

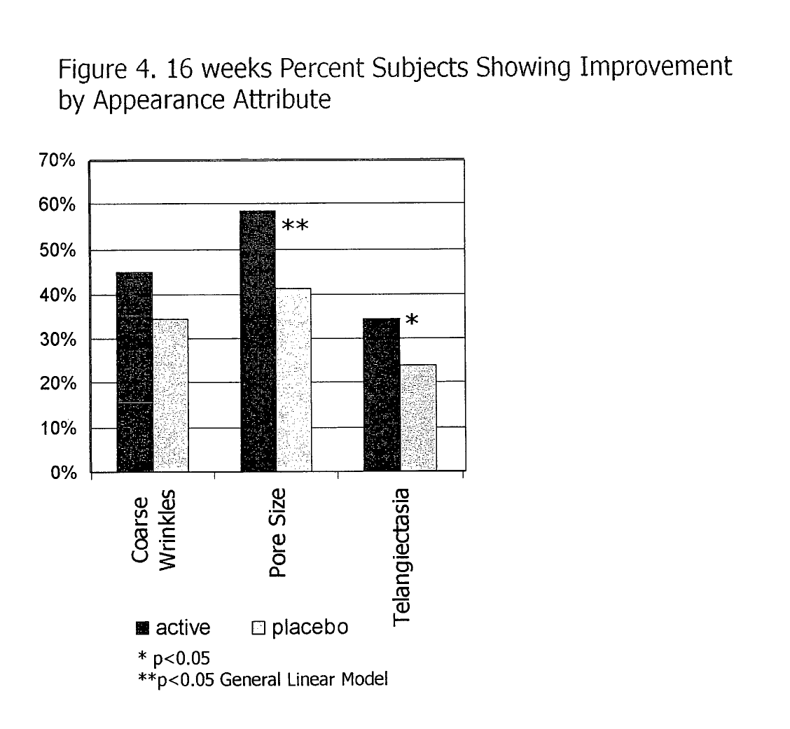

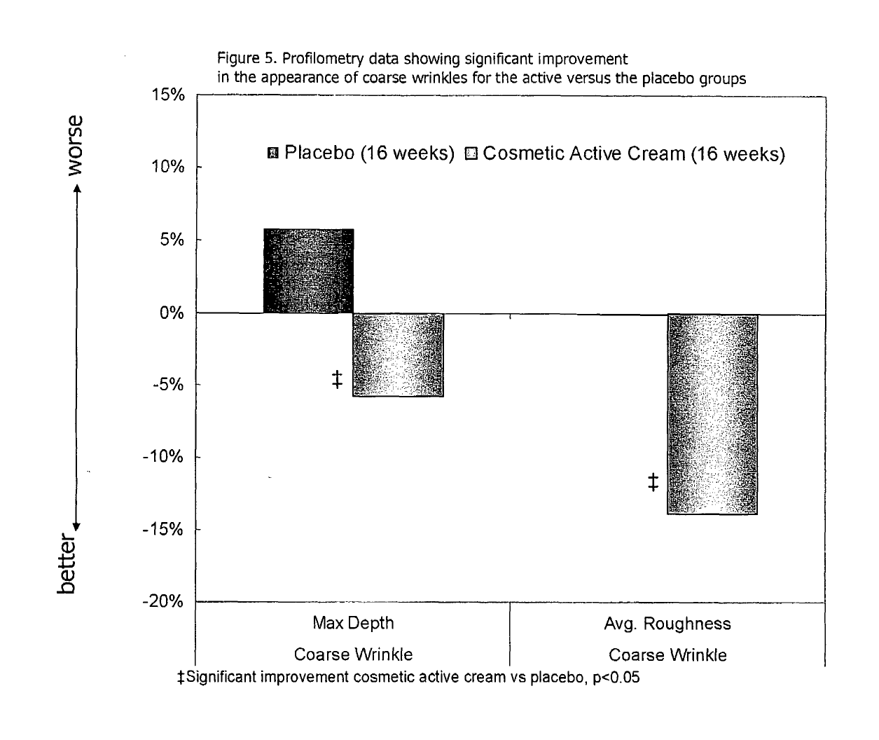

The MMP inhibitor used in the study was found to reduce the appearance of telangiectasia relative to the placebo formulation (p<0.05). As shown in FIG. 1, this is even more remarkable because the control composition consistently caused an increase in the appearance of telangiectasia throughout the 16-week study. In addition, the improved appearance of coarse wrinkles and of pore size between the active and the placebo groups trended toward significance with time. The Proc GLM statistical test demonstrated p-values of 0.06 and 0.02 for the two attributes, respectively, when the interaction between time and treatment was considered. See FIGS. 2-3. The p-value of 0.06 suggested that for the coarse wrinkling attribute, the effect of treatment and treatment time on the final change from the baseline score had a 6% probability of occurring randomly. Similarly, for the pore size attribute, the effect of treatment and treatment time on the outcome had a 2% probability of random occurrence. These p-values indicated that the change in the coarse wrinkling and the pore size scores from the baseline values have a low likelihood of occurring in the absence of the treatment. FIG. 4 provides a summary of the attributes with improved appearance based on the scoring methods used by the dermatologist. Data from profilometry of silicone replica supported the dermatology assessment of coarse wrinkle improvement, where a significant improvement was detected for the treatment group relative to the placebo group (p<0.05 for coarse wrinkle depth and roughness). See FIG. 5.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A cosmetic method of skin care, the method comprising steps of:
applying to the skin of a subject a cosmetically effective amount of a matrix metalloproteinase (MMP) inhibitor in a cosmetically suitable vehicle in order to improve the appearance of skin, wherein the MMP inhibitor is of one of the formulae: 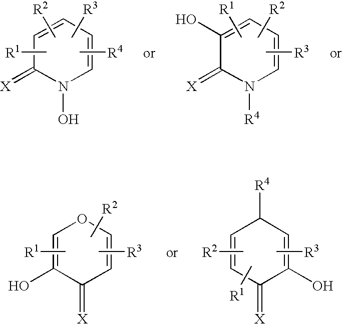
wherein
X is O or S; and
each of R¹, R², R³, and R⁴ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —C(O)NHR_{A}; —CH₂NHR_{A}; —CH₂C(═O)NHR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
and at least one of R¹, R², R³, and R⁴ is not hydrogen; or a cosmetically acceptable form thereof.

2. The cosmetic method of claim 1, wherein the MMP inhibitor is of the formula:

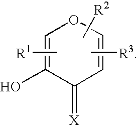

3. The cosmetic method of claim 2, wherein X is O.

4. The cosmetic method of claim 1, wherein the MMP inhibitor is:

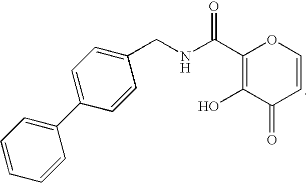

5. The cosmetic method of claim 1, wherein the MMP inhibitor is:

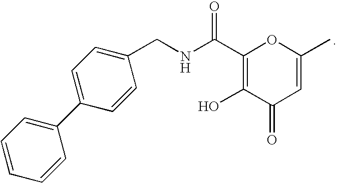

6. The cosmetic method of claim 1, wherein the MMP inhibitor is:

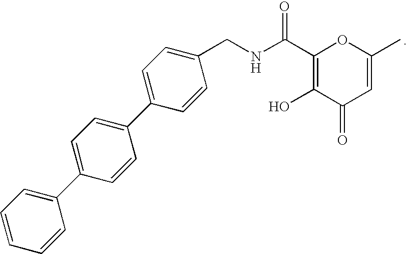

7. The cosmetic method of claim 1, wherein the MMP inhibitor is:

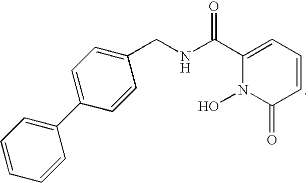

8. The cosmetic method of claim 1, wherein the step of applying is repeated at least daily.

9. The cosmetic method of claim 1, wherein the step of applying is repeated at least twice daily.

10. The cosmetic method of claim 1 further comprising applying to the skin of the subject a retinoid.

11. The cosmetic method of claim 1 further comprising applying to the skin of the subject a sunscreen.

12. The cosmetic method of claim 1 further comprising applying to the skin of the subject an antioxidant.

13. A pharmaceutical method of treating skin, the method comprising steps of:
administering to the skin of a subject a therapeutically effective amount of a matrix metalloproteinase (MMP) inhibitor in a pharmaceutically acceptable excipient in order to treat a skin condition, wherein the MMP inhibitor is of one of the formulae: 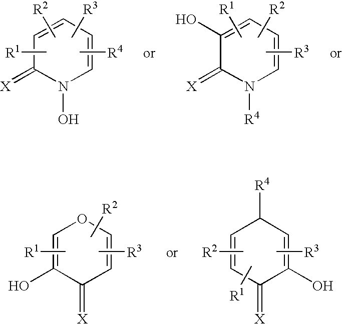
wherein
X is O or S; and
each of R¹, R², R³, and R⁴ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —C(O)NHR_{A}; —CH₂NHR_{A}; —CH₂C(═O)NHR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
and at least one of R¹, R², R³, and R⁴ is not hydrogen; or a pharmaceutically acceptable form thereof.

14. The pharmaceutical method of claim 13, wherein the MMP inhibitor is of the formula:

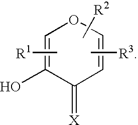

15. The pharmaceutical method of claim 14, wherein X is O.

16. The pharmaceutical method of claim 13, wherein the MMP inhibitor is:

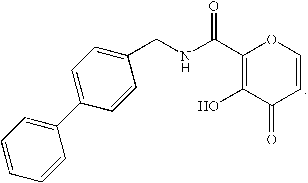

17. The pharmaceutical method of claim 13, wherein the MMP inhibitor is:

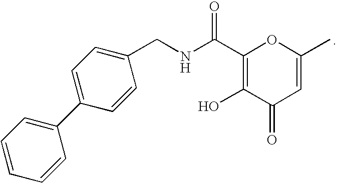

18. The pharmaceutical method of claim 13, wherein the MMP inhibitor is:

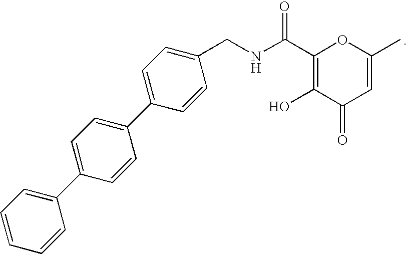

19. The pharmaceutical method of claim 13, wherein the MMP inhibitor is:

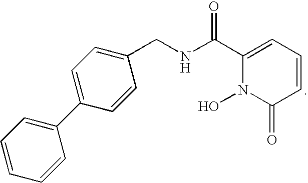

20. The pharmaceutical method of claim 13, wherein the step of administering is repeated at least weekly.

21. The pharmaceutical method of claim 13, wherein the step of administering is repeated at least daily.

22. The pharmaceutical method of claim 13, wherein the step of administering is repeated at least twice daily.

23. The pharmaceutical method of claim 13, wherein the amount of MMP inhibitor applied is effective to decrease the degradation of collagen in the treated skin by at least 25%.

24. The pharmaceutical method of claim 13, wherein the amount of MMP inhibitor applied is effective to decrease the degradation of collagen in the treated skin by at least 50%.

25. The pharmaceutical method of claim 13, wherein the amount of MMP inhibitor applied is effective to decrease the degradation of collagen in the treated skin by at least 75%.

26. The pharmaceutical method of claim 13, wherein the amount of MMP inhibitor applied is effective to decrease the degradation of collagen in the treated skin by at least 90%.

27. The pharmaceutical method of claim 13, wherein the amount of MMP inhibitor applied is effective to increase or prevent the decrease of procollagen levels.

28. The pharmaceutical method of claim 13, wherein the amount of MMP inhibitor applied is effective to stimulate the formation of new collagen.

29. A pharmaceutical method of treating telangiectasia, the method comprising:
administering to the skin of a subject an amount of a matrix metalloproteinase (MMP) inhibitor sufficient to reduce the appearance of telangiectasia, wherein the MMP inhibitor is of one of the formulae: 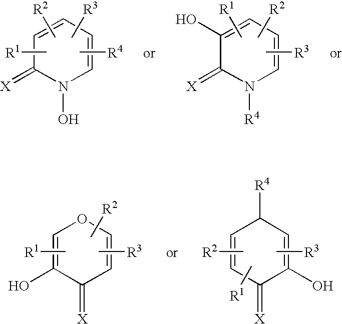
wherein
X is O or S; and
each of R¹, R², R³, and R⁴ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —C(O)NHR_{A}; —CH₂NHR_{A}; —CH₂C(═O)NHR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
and at least one of R¹, R², R³, and R⁴ is not hydrogen; or a pharmaceutically acceptable form thereof.

30. The pharmaceutical method of claim 29, wherein the reduction in the appearance of telangiectasia is measured using scored visual assessment by a dermatologist.

31. The pharmaceutical method of claim 29, wherein the MMP inhibitor is of the formula:

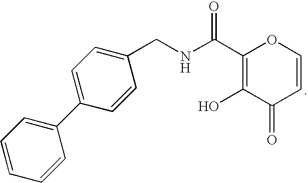

32. A pharmaceutical method of treating coarse wrinkles, the method comprising:
administering to the skin of a subject an amount of a matrix metalloproteinase (MMP) inhibitor sufficient to reduce the appearance of coarse wrinkles, wherein the MMP inhibitor is of one of the formulae: 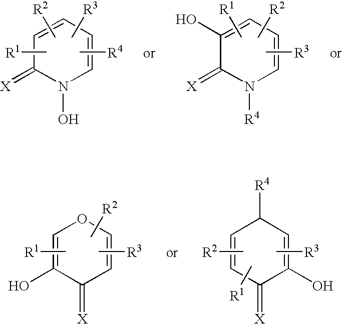
wherein
X is O or S; and
each of R¹, R², R³, and R⁴ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —C(O)NHR_{A}; —CH₂NHR_{A}; —CH₂C(═O)NHR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
and at least one of R¹, R², R³, and R⁴ is not hydrogen; or a pharmaceutically acceptable form thereof.

33. The pharmaceutical method of claim 32, wherein the reduction in the appearance of coarse wrinkles is measured by profilometry of silicone replicas from the crow's feet area of the subject's face.

34. The pharmaceutical method of claim 32, wherein the reduction in the appearance of coarse wrinkles is measured using scored visual assessment by a dermatologist.

35. The pharmaceutical method of claim 32, wherein the MMP inhibitor is of the formula:

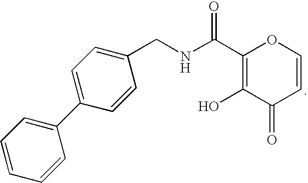

36. A cosmetic method decreasing the appearance of pore size in the skin, the method comprising:
administering to the skin of a subject an amount of a matrix metalloproteinase (MMP) inhibitor sufficient to reduce the apparent pore size, wherein the MMP inhibitor is of one of the formulae: 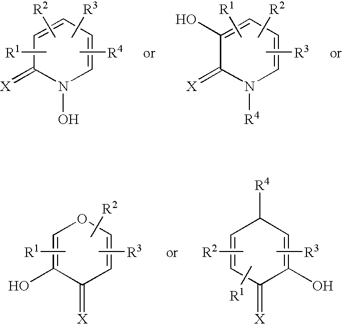
wherein
X is O or S; and
each of R¹, R², R³, and R⁴ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —C(O)NHR_{A}; —CH₂NHR_{A}; —CH₂C(═O)NHR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
and at least one of R¹, R², R³, and R⁴ is not hydrogen; or a cosmetically acceptable form thereof.

37. The cosmetic method of claim 36, wherein the appearance in reduction in pore size is measured using scored visual assessment by a dermatologist.

38. The cosmetic method of claim 36, wherein the MMP inhibitor is of the formula:

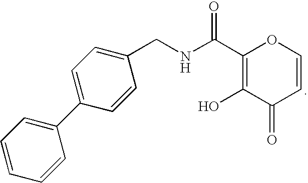

39. A cosmetic method of reducing the appearance of redness, the method comprising:
administering to the skin of a subject an amount of a matrix metalloproteinase (MMP) inhibitor sufficient to reduce the appearance of redness, wherein the MMP inhibitor is of one of the formulae: 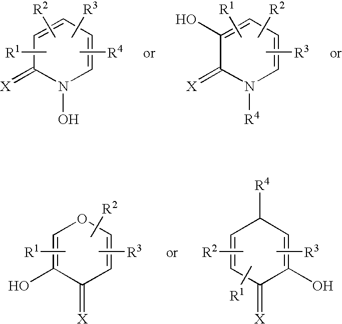
wherein
X is O or S; and
each of R¹, R², R³, and R⁴ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —C(O)NHR_{A}; —CH₂NHR_{A}; —CH₂C(═O)NHR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
and at least one of R¹, R², R³, and R⁴ is not hydrogen; or a cosmetically acceptable form thereof.

40. The cosmetic method of claim 39, wherein the reduction in the appearance of redness is measured using scored visual assessment by a dermatologist.

41. The cosmetic method of claim 39, wherein the MMP inhibitor is of the formula:

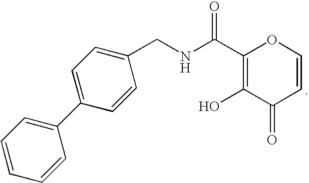

42. A cosmetic method of reducing the appearance of coarse wrinkles, the method comprising:
administering to the skin of a subject an amount of a matrix metalloproteinase (MMP) inhibitor sufficient to reduce the appearance of coarse wrinkles.

43. The cosmetic method of claim 42, wherein the reduction in the appearance of coarse wrinkles is measured by profilometry of silicone replicas from the crow's feet area of the subject's face.

44. The cosmetic method of claim 42, wherein the reduction in the appearance of coarse wrinkles is measured using scored visual assessment by a dermatologist.

45. The cosmetic method of claim 42, wherein the MMP inhibitor is of the formula:

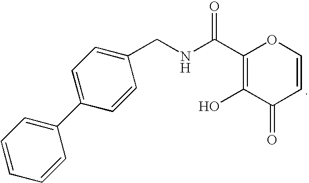

46. A cosmetic method of reducing the appearance of pores in the skin, the method comprising:
administering to the skin of a subject an amount of a matrix metalloproteinase (MMP) inhibitor sufficient to reduce the appearance of pores in the skin.

47. The cosmetic method of claim 46, wherein the reduction in appearance of pores is measured using scored visual assessment by a dermatologist.

48. The cosmetic method of claim 46, wherein the MMP inhibitor is of the formula:

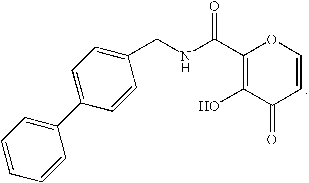

49. A cosmetic composition comprising:
a matrix metalloproteinase (MMP) inhibitor, wherein the MMP inhibitor is of one of the formulae: 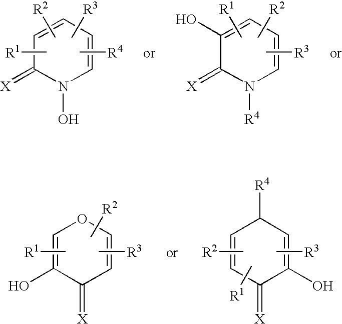
wherein
X is O or S; and
each of R¹, R², R³, and R⁴ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —C(O)NHR_{A}; —CH₂NHR_{A}; —CH₂C(═O)NHR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
and at least one of R¹, R², R³, and R⁴ is not hydrogen; or a pharmaceutically acceptable form thereof; and a cosmetically acceptable excipient.

50. The cosmetic composition of claim 49, wherein the composition is in the form selected from the group consisting of lotions, creams, gels, solutions, pastes, serums, solid sticks, powders, sprays, face mask, and foams.

51. The cosmetic composition of claim 49, wherein the cosmetically acceptable vehicle comprises a solubilizer.

52. The cosmetic composition of claim 51, wherein the solubilizer is selected from the group consisting of ether, alkoxylated alcohols, alkoxylated amines, sorbitan esters, phospholipids, and fatty quaternaries.

53. The cosmetic composition of claim 51, wherein the solubilizer is dimethyl isosorbide.

54. The cosmetic composition of claim 51, wherein the solubilizer is polyethylene glycol.

55. The cosmetic composition of claim 51, wherein the solubilizer is a phospholipid.

56. The cosmetic composition of claim 51, wherein the solubilizer is a fatty quaternary amine.

57. The cosmetic composition of claim 49, wherein the cosmetically acceptable vehicle comprises an emulsifier.

58. The cosmetic composition of claim 57, wherein the emulsifier is an anionic emulsifier.

59. The cosmetic composition of claim 57, wherein the emulsifier is a cationic emulsifier.

60. The cosmetic composition of claim 57, wherein the emulsifier is a non-ionic emulsifier.

61. The cosmetic composition of claim 49, wherein the cosmetically acceptable vehicle comprises an oil, lipid, phospholipids, silicone, or wax.

62. The cosmetic composition of claim 49, wherein the cosmetically acceptable vehicle comprises a fatty acid.

63. The cosmetic composition of claim 49, wherein the cosmetically acceptable vehicle comprises water, an alcohol, or a solvent.

64. The cosmetic composition of claim 49, wherein the cosmetically acceptable vehicle comprises a synthetic polymer.

65. The cosmetic composition of claim 49, wherein the cosmetically acceptable vehicle comprises a humectant, emollient, lubricant, wetting agent, or thickening agent.

66. The cosmetic composition of claim 49 further comprising a sunscreen.

67. The cosmetic composition of claim 49 further comprising an antioxidant, a reducing agent, a chelating agent or preservative.

68. The cosmetic composition of claim 67, wherein the antioxidant is selected from the group consisting of butylated hydroxytoluene (BHT), ascorbyl palmitate (vitamin C palmitate), and tocopherol.

69. The cosmetic composition of claim 49 further comprising a vitamin.

70. The cosmetic composition of claim 49 further comprising a fragrance.

71. The cosmetic composition of claim 49 further comprising a plant extract.

72. The cosmetic composition of claim 49 further comprising a coloring agent.

73. The cosmetic composition of claim 49 further comprising a protein, peptide, or amino acid

74. The cosmetic composition of claim 49 further comprising a polysaccharide, oligosaccharide, or sugar.

75. The cosmetic composition of claim 49 further comprising a polynucleotide, nucleoside or nucleotide.

76. The cosmetic composition of claim 49, wherein the MMP inhibitor is encapsulated in a polymeric matrix.

77. The cosmetic composition of claim 49, wherein the MMP inhibitor is encapsulated in polymeric particles.

78. The cosmetic composition of claim 49, wherein the MMP inhibitor is in a liposome or micelle.

79. A cosmetic composition comprising water, disodium EDTA, carbomer, xantham gum, PEG-7 glyceryl cocoate, glycerin, butylated hydroxytoluene, dimethyl isosorbide, ceteraryl alcohol ceteareth-20, capryl/capric triglyceride, glyceryl stearate PEG-100 stearate, sodium hydroxide, phenoxyethanol, chlorphenesin, methylparaben, propylparaben, and an MMP inhibitor of formula:

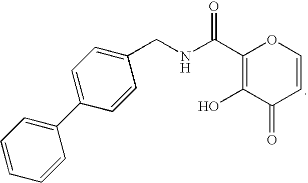

80. A cosmetic composition comprising water, glycerin, sodium chloride, hyaluronic acid, echinacin, ascorbyl palmitate, tocopheryl acetate, BHT, PEG-4, PEG-7 glyceryl cocoate, cyclopentasiloxane, polysilicone 11, glyceryl laurate, cetyl PEG/PPG 10/1 dimethicone, PEG/PPG-18/18 dimethicone, dimethicone, vinyl dimethicone cross polymer, silica, phenoxyethanol, caprylyl glycol, sorbic acid, and an MMP inhibitor of formula:

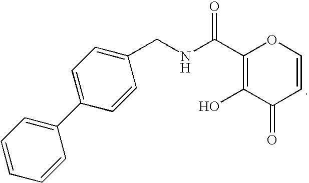

81. A cosmetic composition comprising water, an antioxidant, a solubilizer, and an MMP inhibitor of formula:

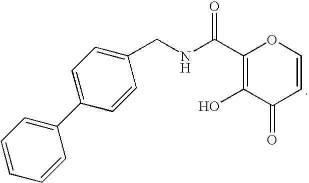

82. A method of treating or preventing a skin disease, the method comprising steps of:
administering to a subject a therapeutically effective amount of a matrix metalloproteinase (MMP) inhibitor optionally in combination with a pharmaceutically acceptable excipient, wherein the MMP inhibitor is of one of the formulae: 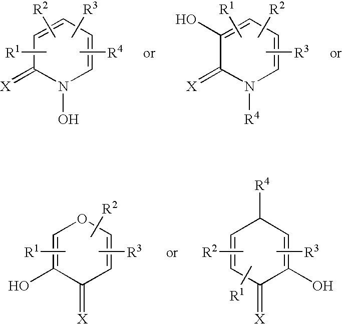
wherein
X is O or S; and
each of R¹, R², R³, and R⁴ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —C(O)NHR_{A}; —CH₂NHR_{A}; —CH₂C(═O)NHR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
and at least one of R¹, R², R³, and R⁴ is not hydrogen; or a pharmaceutically acceptable form thereof.

83. The method of claim 82, wherein the subject is a human.

84. The method of claim 82, wherein the step of administering comprises administering the therapeutically effective amount of a matrix metalloproteinase (MMP) inhibitor to the skin of the subject.

85. The method of claim 82, wherein the skin disease is an inflammatory disease.

86. The method of claim 82, wherein the skin disease is an autoimmune disease.

87. The method of claim 82, wherein the skin disease is a proliferative disease.

88. The method of claim 82, wherein the skin disease is skin cancer.

89. The method of claim 82, wherein the skin disease is a wound.

90. The method of claim 82, wherein the MMP inhibitor is of the formula:

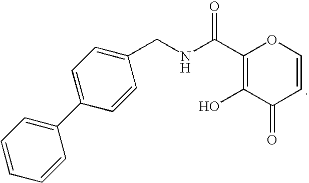

91. A pharmaceutical composition comprising:
a matrix metalloproteinase (MMP) inhibitor, wherein the MMP inhibitor is of one of the formulae: 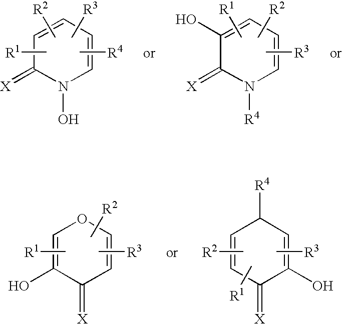
wherein
X is O or S; and
each of R¹, R², R³, and R⁴ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —C(O)NHR_{A}; —CH₂NHR_{A}; —CH₂C(═O)NHR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
and at least one of R¹, R², R³, and R⁴ is not hydrogen; or a pharmaceutically acceptable form thereof; and
a pharmaceutically acceptable excipient.

92. The pharmaceutical composition of claim 91, wherein the composition is suitable for transdermal administration of the MMP inhibitor.
